(19) United States
(12) Patent Application Publication
(10) Pub. No.: US 2024/0376092 A1
(43) Pub. Date: Nov. 14, 2024

(54) MODULATORS OF PrPC AND USES THEREOF

(71) Applicants: FONDAZIONE TELETHON ETS, Roma (IT); UNIVERSITA' DEGLI STUDI DI PERUGIA, Perugia (IT); UNIVERSITA' DEGLI STUDI DI TRENTO, Trento (IT)

(72) Inventors: Emiliano Biasini, Trento (IT); Maria Letizia Barreca, Perugia (IT); Giuseppe Manfroni, Perugia (IT); Francesca Fallarino, Perugia (IT)

(73) Assignees: FONDAZIONE TELETHON ETS, ROMA (IT); UNIVERSITA' DEGLI STUDI DI PERUGIA, Perugia (IT); UNIVERSITA' DEGLI STUDI DI TRENTO, Trento (IT)

(21) Appl. No.: 18/562,896

(22) PCT Filed: May 20, 2022
(86) PCT No.: PCT/EP2022/063806

§ 371 Date: Nov. 21, 2023

(30) Foreign Application Priority Data
May 21, 2021 (IT) 102021000013244

(51) Int. Cl. C07D 417/12; A61K 31/5415; A61K 31/542; A61P 25/28; C07D 279/02; C07D 417/04; C07D 513/04

(52) U.S. Cl. CPC C07D 417/12; A61K 31/5415; A61K 31/542; A61P 25/28; C07D 279/02; C07D 417/04; C07D 513/04

(57) ABSTRACT

The present invention relates to compounds capable of modulating the activity of the cellular prion protein (PrPC) and their use for the treatment of immune and neurodegenerative diseases.

### FIELD OF THE INVENTION

The present invention relates to compounds capable of modulating the activity of the cellular prion protein (PrPC) and their use for the treatment of neurodegenerative and immune diseases. In particular the compounds of the invention are useful in the treatment of Alzheimer Disease, Prion Disease, Multiple Sclerosis, Autoimmune Encephalitis, Parkinson's Disease, Inflammatory Bowel Disease and Crohn's disease.

### BACKGROUND OF THE INVENTION

Aging is linked to a wide range of molecular, cellular and functional changes, which particularly affect the integrity of the nervous system. One fundamental process altered by aging is protein folding. When proteins misfold, they acquire alternative conformations capable of seeding a cascade of molecular events, ultimately resulting in neuronal dysfunction and death. Indeed, a wide range of age-related disorders is linked to protein misfolding and aggregation in the brain. Examples include common disorders such as Parkinson's and Alzheimer's diseases, as well as rarer disorders such as prion diseases¹. Alzheimer's disease is the most common form of dementia in the elderly population, currently affecting almost 36 million individuals worldwide. The number will increase dramatically in the coming decades as the population ages, producing devastating medical and socio-economic consequences. According to the amyloid cascade hypothesis, Alzheimer's disease is a consequence of the accumulation in the brain of the 40-42 amino acid Aβ peptide, a cleavage product of the amyloid precursor protein (APP). The majority of Alzheimer's disease cases manifest as a late onset, sporadic form. However, approximately 5% of cases are inherited in an autosomal dominant fashion. These forms, collectively referred to as familial Alzheimer's diseases, are linked to at least 230 mutations in genes encoding for APP or the presenilins (PS1 or PS2)². The mutations are thought to favor the accumulation of Aβ peptide in the brain, by increasing its production or reducing its clearance. The Aβ peptide spontaneously forms polymers ranging from small, soluble oligomers to large, insoluble fibrils. A great deal of evidence suggests that soluble Aβ oligomers, rather than fibrillar aggregates, are primarily responsible for the synaptic dysfunction underlying the cognitive decline in Alzheimer's disease³. Aβ oligomers are believed to act by binding to cell surface receptors that transduce their detrimental effects on synapses. The identification of such receptor sites has important therapeutic implications, as they represent potential targets for pharmacological intervention. Recently, a novel candidate has emerged as a receptor for Aβ oligomers: the cellular form of the prion protein (PrPC)⁴. PrPC, an endogenous, cell-surface glycoprotein of unknown function, plays a central role in transmissible neurodegenerative disorders commonly referred to as prion diseases. PrPC was originally discovered for its central role in transmissible spongiform encephalopathies (also called prion diseases) and has been claimed to participate in several other pathologies of the nervous system, including Alzheimer's and Parkinson's diseases, by acting as a toxicity-transducing receptor for different misfolded protein isoforms. Interestingly, PrPC has also been reported to exert important functions outside the nervous system as well, in particular in the immune system, and the protein has emerged as a key factor for myelin homeostasis. Consistent with these concepts, additional studies have revealed that the absence of PrPC exacerbates inflammatory damage in a variety of laboratory models of brain ischemia, brain trauma, experimental autoimmune encephalomyelitis (EAE), and experimental colitis.

Prion diseases, which can manifest in a sporadic, inherited or acquired fashion, are caused by the conformational conversion of PrPC into a misfolded isoform (called scrapie form of PrP, or PrPSc) that accumulates in the central nervous system of affected individuals. PrPSc is an infectious protein (prion) that propagates itself by binding to PrPC, triggering its conformational rearrangement into new PrPSc molecules⁵. A great deal of evidence indicates a distinction between prion infectivity and toxicity, and suggested that a physiological function of PrPC may be altered upon binding to PrPSc, to deliver neurotoxic signals. In fact, genetically depleting neuronal PrPC in prion-infected mice has been shown to reverse neuronal loss and clinical progression, despite the continuous production of PrPSc by surrounding astrocytes⁶. Thus, the presence of PrPC on the neuronal surface is critical not only for supporting PrPSc propagation, but also for transducing its neurotoxicity^{7,8}. This conclusion recently found unexpected support from data involving Aβ oligomers. PrPC emerged from an expression cloning screen as a receptor capable of binding Aβ oligomers with nanomolar affinity. Importantly, PrPC was also found to be a mediator of Aβ-induced synaptotoxicity⁴. In support of this conclusion, hippocampal slices derived from PrP knockout (KO) mice were shown to be resistant to Aβ oligomer-induced suppression of long-term potentiation (LTP), an in vitro correlate of memory and synaptic function. Consistent with this, application of anti-PrP antibodies was shown to prevent Aβ-induced synaptic dysfunction in hippocampal slices⁹. Finally, PrPC was required for both the cognitive deficits and reduced survival observed in transgenic mouse models of Alzheimer's disease¹⁰. A number of subsequent studies have extended this observation by discovering that several Aβ assemblies, including neurotoxic Aβ oligomers, bind with high affinity to PrPC via two sites in the unstructured, N-terminal tail of the protein (residues 23-27 and 95-105)¹¹. This interaction unleashes a toxic signalling involving the metabotropic glutamate receptor 5 (mGluR5), activation of the tyrosine kinase Fyn, and phosphorylation of the NR2B subunit of NMDA receptors, ultimately producing dysregulation of receptor function, excitoxicity and dendritic spine retraction². Other recent studies provided evidence that PrPC could mediate the toxicity not only of Aβ oligomers, but also of other 3-sheet-rich protein conformers, including alpha synuclein, involved in Parkinson disease¹³⁻¹⁵. These results indicate that misfolded assemblies of several different pathogenic proteins could exert their effects by blocking, enhancing or altering the normal activity of PrPC⁸. The conclusion highlights a close connection between the role of PrPC in several neurodegenerative diseases and its physiological function. Several activities have been attributed to PrPC in the nervous system, mostly based on subtle abnormalities detected in mice or cells depleted for PrPC. These include roles in neuroprotection, synaptic integrity, neuronal excitability and memory formation¹⁶. Recently, PrPC has been also shown to play important functions outside the nervous system as well, in particular in the immune system¹⁷. PrPC appears to be protective in autoimmune colitis. Inflammatory bowel disease, induced by dextran sodium sulphate (DSS), is more severe in PrPO/O mice than in wild-type mice. Accordingly, overexpression of PrPC greatly attenuates DSS-induced colitis. Upon MHIC/peptide-driven interactions between T cells and dendritic cells (DCs), PrPC migrates to the immunological synapse and exerts differential effects on T cell proliferation and cytokine production, as revealed by ablation or antibody masking on the DCs or on the lymphocyte side of the synapse¹⁸. DCs are professional APCs and also very plastic cells that play an important role in T helper (Th) cells differentiation and thus are involved in the induction of both autoimmunity and tolerance¹⁹. Surprisingly, authors of the invention found that selected DC subsets express high level PrPC. Recent data have also shown that EAE is worsened in mice lacking PrPC, indicating that this protein may act as a regulatory molecule, and that cells lacking PrPC may become more inflammatory and behave more aggressively against the central nervous system. These results led us to hypothesize that targeting PrPC pharmacologically may activate protective immunoregulatory effects in MS.

A highly robust and quick assay to detect the spontaneous toxicity of mutant PrPC in cell cultures, named the cell-based drug assay (DBCA), has been previously described²⁰. This novel assay was recently employed to identify small molecules capable of abrogating mutant PrPC activity^{21,22}. Importantly, derivatives of one of such molecules (called SMs) arising from several cycles of chemical optimization rescued Aβ-induced synaptic dysfunction in primary hippocampal neurons, and rescued electrophysiological abnormalities induced by prions in mouse brain slices. Collectively, these data have led us to hypothesize that the pharmacological modulation of PrPC activity might confer therapeutic benefit in multiple sclerosis (MS), a neurodegenerative disorder characterized by progressive myelin loss. Moreover, in a mouse model of EAE, it was found that systemic administration of such PrPC modulators resulted in significant reduction of disease severity, compared to untreated controls. Within this conceptual framework, there is still the need for compounds capable of modulating the activity of PrPC.

### SUMMARY OF INVENTION

In the present invention it was surprisingly found that a properly functionalized thiazine-dioxide scaffold provides a series of compounds capable of abrogating mutant PrPC activity. The results obtained within the present invention clearly demonstrate that compounds may represent new therapeutic options for several different pathologies, such as prion and Alzheimer's diseases, autoimmune encephalitis and MS.

It is an object of the present invention a compound of general Formula (I):

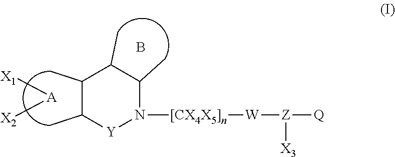

wherein
A is a benzene ring or a five- or six heteroaromatic ring;
B is a benzene ring of general structure:

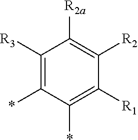

Or B is a five- or six membered heteroaromatic ring optionally substituted by one or more substituents each independently selected from hydrogen, halogen, nitro, cyano, thiol, C₁₋₄ alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino;
W is C(═O), C(═S), CH₂ or is absent;
Y is selected from CH₂, SO₂, SO, S, C(═O), PO₂, and NR₄; preferably Y is selected from CH₂, SO₂, SO, C(═O), and NR₄;
Z is N or CH;
X₁ and X₂ are each independently selected in each instance from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino, OH, pyrrolidine, piperidine, morpholine, piperazine, N-methylpiperazine;
X₃ is hydrogen, methyl, ethyl, isopropyl or benzyl;
X₄ and X₅ are independently selected in each instance from hydrogen, C₁₋₃alkyl, haloalkyl, halogen, cycloalkyl, amino, hydroxy, cyano, nitro; n is 0, 1, 2, 3, 4;
or residues X₃ and X₄ taken together represent a single bond or a C₁₋₄alkanediyl, said single bond or said C₁₋₄alkanediyl forming together with the bridging atoms to which they are respectively linked a 5 or 6 membered heterocyclic ring;
R₁, R₂, R₂ₐ and R₃ are each independently selected from hydrogen, halogen, nitro, cyano, hydroxy, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, OC₁₋₄alkylamino, SC₁₋₄alkyl;
R₄ is selected from hydrogen, C₁₋₄alkyl, C₁₋₄aminoalkyl, C₁₋₄hydroxyalkyl, C₁₋₄nitroalkyl, C₁₋₄thioalkyl, C₁₋₆haloalkyl;
Q is selected from C₁₋₈alkyl, C₁₋₈alkenyl, cycloalkyl, heterocycloalkyl, aryl ring, heteroaromatic ring, wherein:
   the C₁₋₈alkyl is optionally substituted with hydroxy, OC₁₋₄alkyl, NHC₁₋₄alkyl, N(C₁₋₄alkyl)₂, NH(C═O)C₁₋₄alkyl, aryl, heteroaryl, heterocycloalkyl, cycloalkyl, cycloalkenyl, each of said aryl, heteroaryl, heterocycloalkyl, cycloalkyl, cycloalkenyl being optionally substituted with methyl, halogen, hydroxy;
   the cycloalkyl and the heterocycloalkyl are each optionally substituted with OH, OSO₂R₅, C₁₋₃alkyl, NR₆R₇, wherein:
      R₅ is selected from hydrogen, phenyl, heteroaryl, aminophenyl and nitrophenyl; and wherein
      R₆ and R₇ are each independently selected from H, methyl, C(═O)CH₃, SO₂CH₃;
   the aryl ring or the heteroaromatic ring are each optionally substituted with one or more substituents selected from halogen, nitro, cyano, thiol, C₁₋₄alkyl, haloalkyl, O-haloalkyl, OC₁₋₄alkyl, NH₂, NHSO₂C₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino;
      and any stereoisomer, pharmaceutically acceptable salt, solvate, hydrate thereof for use in the treatment of a neurodegenerative disease or an immune disease, preferably for use in the treatment of Alzheimer Disease, Prion Disease, Multiple Sclerosis, Autoimmune Encephalitis, Parkinson's Disease, Inflammatory Bowel Disease, Crohn's disease, provided that compound

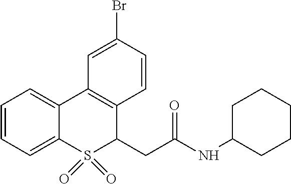 [SMILES: O=C(CC1c2ccc(Br)cc2-c2ccccc2S1(=O)=O)NC1CCCCC1]

is not included.

It is a further object of the invention a compound of formula (I)

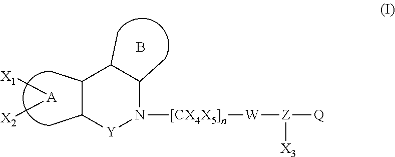

wherein
A is a benzene ring or a five- or six heteroaromatic ring;
B is a benzene ring of general structure:

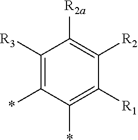

Or B is a five- or six membered heteroaromatic ring optionally substituted by one or more substituents each independently selected from hydrogen, halogen, nitro, cyano, thiol, C₁₋₄ alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino;
W is C(═O), C(═S), CH₂ or is absent;
Y is selected from CH₂, SO₂, SO, S, C(═O), PO₂, and NR₄; preferably Y is selected from CH₂, SO₂, SO, C(═O), and NR₄;
Z is N or CH;
X₁ and X₂ are each independently selected in each instance from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino, OH, pyrrolidine, piperidine, morpholine, piperazine, N-methylpiperazine;
X₃ is hydrogen, methyl, ethyl, isopropyl or benzyl;
X₄ and X₅ are independently selected in each instance from hydrogen, C₁₋₃alkyl, haloalkyl, halogen, cycloalkyl, amino, hydroxy, cyano, nitro; n is 0, 1, 2, 3, 4;
or residues X₃ and X₄ taken together represent a single bond or a C₁₋₄alkanediyl, said single bond or said C₁₋₄alkanediyl forming together with the bridging atoms to which they are respectively linked a 5 or 6 membered heterocyclic ring;
R₁, R₂, R₂ₐ and R₃ are each independently selected from hydrogen, halogen, nitro, cyano, hydroxy, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, OC₁₋₄alkylamino, SC₁₋₄alkyl;
R₄ is selected from hydrogen, C₁₋₄alkyl, C₁₋₄aminoalkyl, C₁₋₄hydroxyalkyl, C₁₋₄nitroalkyl, C₁₋₄thioalkyl, C₁₋₆haloalkyl;
Q is selected from C₁₋₈alkyl, C₁₋₈alkenyl, cycloalkyl, heterocycloalkyl, aryl ring, heteroaromatic ring, wherein:
   the C₁₋₈alkyl is optionally substituted with hydroxy, OC₁₋₄alkyl, NHC₁₋₄alkyl, N(C₁₋₄alkyl)₂, NH(C═O)C₁₋₄alkyl, aryl, heteroaryl, heterocycloalkyl, cycloalkyl, cycloalkenyl, each of said aryl, heteroaryl, heterocycloalkyl, cycloalkyl, cycloalkenyl being optionally substituted with methyl, halogen, hydroxy;
   the cycloalkyl and the heterocycloalkyl are each optionally substituted with OH, OSO₂R₅, C₁₋₃alkyl, NR₆R₇, wherein:
      R₅ is selected from hydrogen, phenyl, heteroaryl, aminophenyl and nitrophenyl; and wherein
      R₆ and R₇ are each independently selected from H, methyl, C(═O)CH₃, SO₂CH₃;
   the aryl ring or the heteroaromatic ring are each optionally substituted with one or more substituents selected from halogen, nitro, cyano, thiol, C₁₋₄alkyl, haloalkyl, O-haloalkyl, OC₁₋₄alkyl, NH₂, NHSO₂C₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino;
      and any stereoisomer, pharmaceutically acceptable salt, hydrate, solvate thereof for use in the treatment of Multiple Sclerosis, Autoimmune Encephalitis or an immune disease, preferably wherein said immune disease is selected from Inflammatory Bowel Disease and Crohn's disease.

Preferably, in the compound of formula (I), A is benzene; and/or Y is SO₂; and/or W is C(═O) or CH₂; and/or Z is N; and/or X₄ and X₅ are H.

Still preferably the compound for use according to the invention has general formula (II):

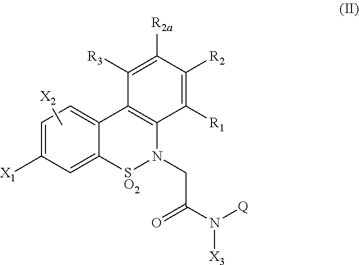

Wherein X₁, X₂, X₃, R₁, R₂, R₂ₐ, R₃ and Q are as defined above.

In a preferred embodiment the compound for use according to the invention is selected form the list below:

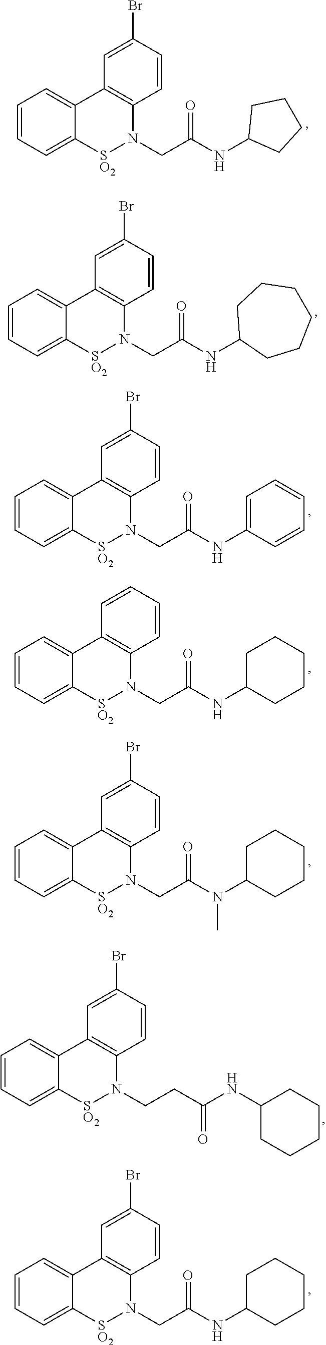

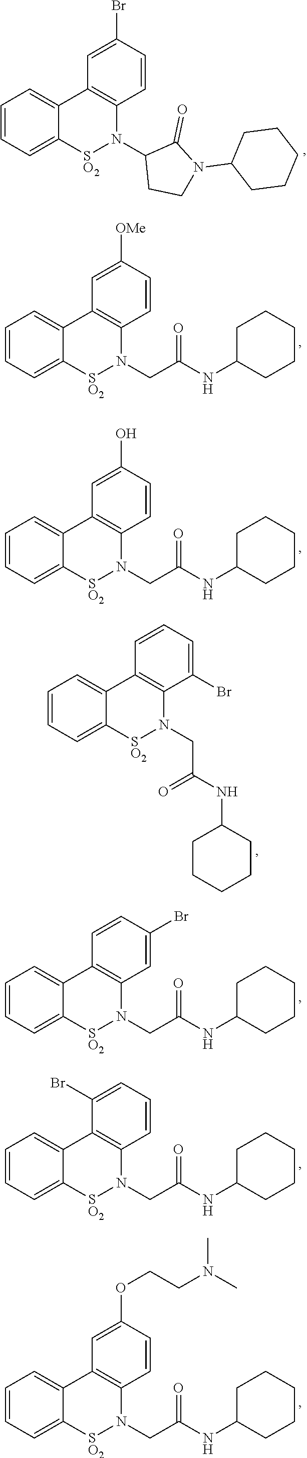

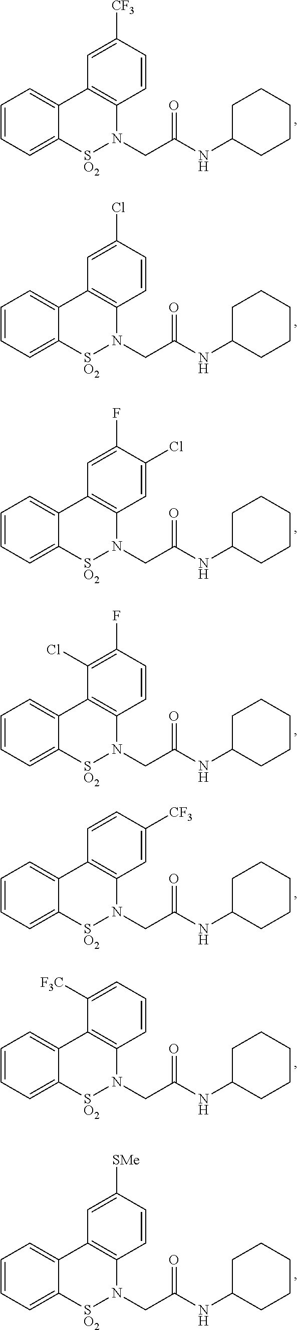

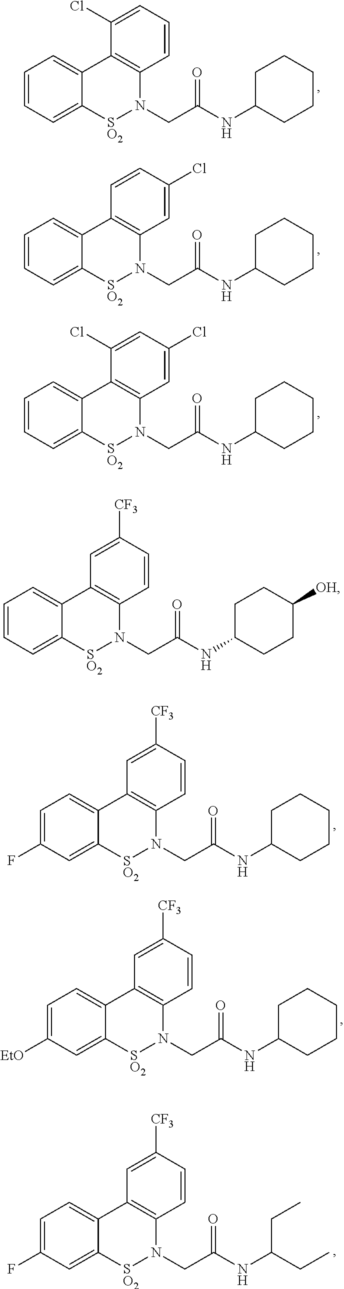

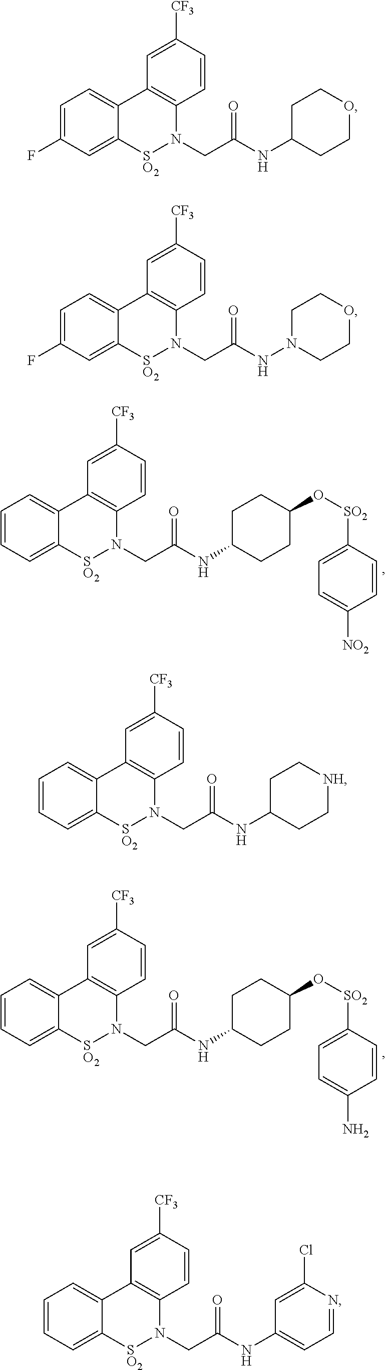 [SMILES: O=C(CN1c2ccc(C(F)(F)F)cc2-c2ccc(F)cc2S1(=O)=O)NC1CCOCC1 ; O=C(CN1c2ccc(C(F)(F)F)cc2-c2ccc(F)cc2S1(=O)=O)NN1CCOCC1 ; O=C(CN1c2ccc(C(F)(F)F)cc2-c2ccccc2S1(=O)=O)N[C@H]1CC[C@H](OS(=O)(=O)c2ccc([N+](=O)[O-])cc2)CC1 ; O=C(CN1c2ccc(C(F)(F)F)cc2-c2ccccc2S1(=O)=O)NC1CCNCC1 ; Nc1ccc(S(=O)(=O)O[C@H]2CC[C@H](NC(=O)CN3c4ccc(C(F)(F)F)cc4-c4ccccc4S3(=O)=O)CC2)cc1 ; O=C(CN1c2ccc(C(F)(F)F)cc2-c2ccccc2S1(=O)=O)Nc1ccnc(Cl)c1]

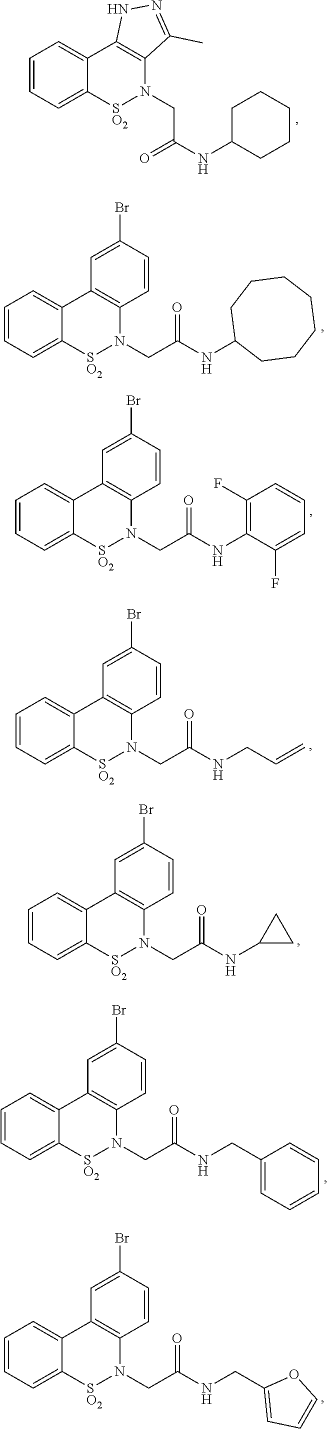

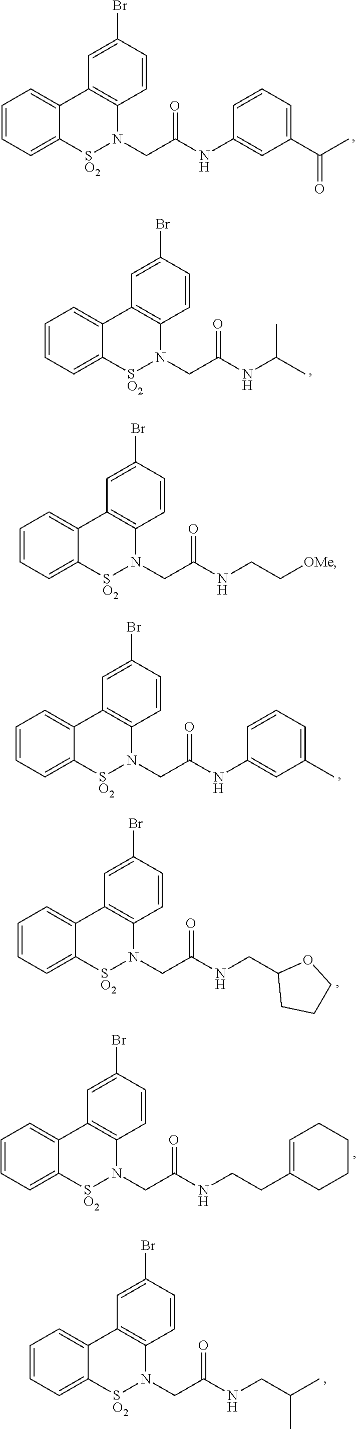

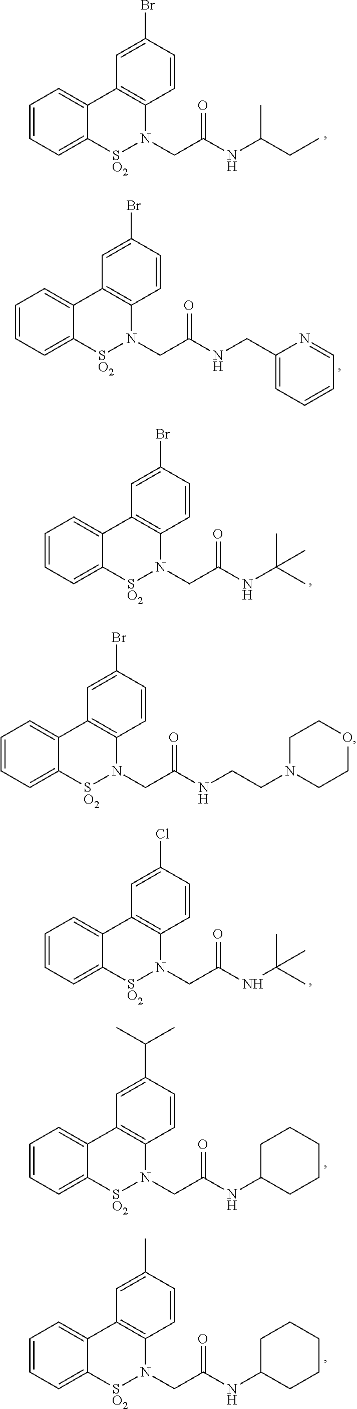

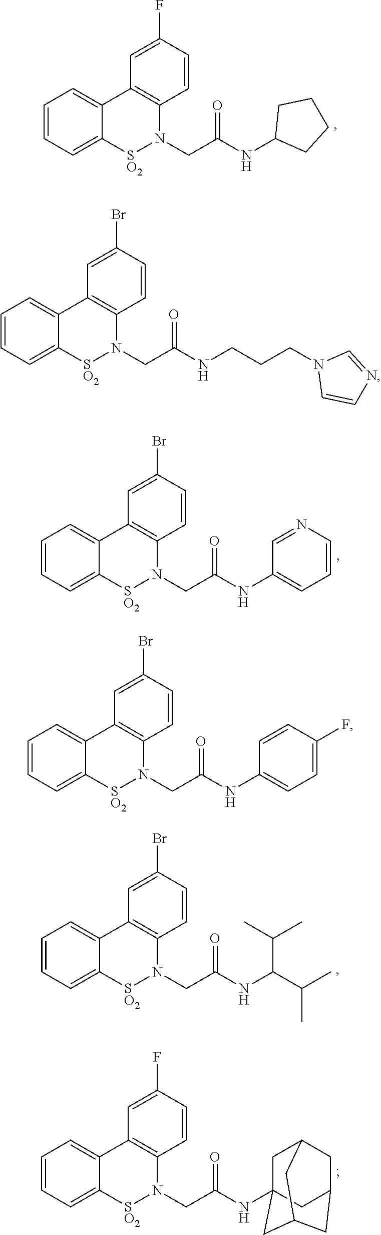 [SMILES: O=C(CN1c2ccc(F)cc2-c2ccccc2S1(=O)=O)NC1CCCC1 ; O=C(CN1c2ccc(Br)cc2-c2ccccc2S1(=O)=O)NCCCn1ccnc1 ; O=C(CN1c2ccc(Br)cc2-c2ccccc2S1(=O)=O)Nc1cccnc1 ; O=C(CN1c2ccc(Br)cc2-c2ccccc2S1(=O)=O)Nc1ccc(F)cc1 ; CC(C)C(NC(=O)CN1c2ccc(Br)cc2-c2ccccc2S1(=O)=O)C(C)C ; O=C(CN1c2ccc(F)cc2-c2ccccc2S1(=O)=O)NC12CC3CC(CC(C3)C1)C2]

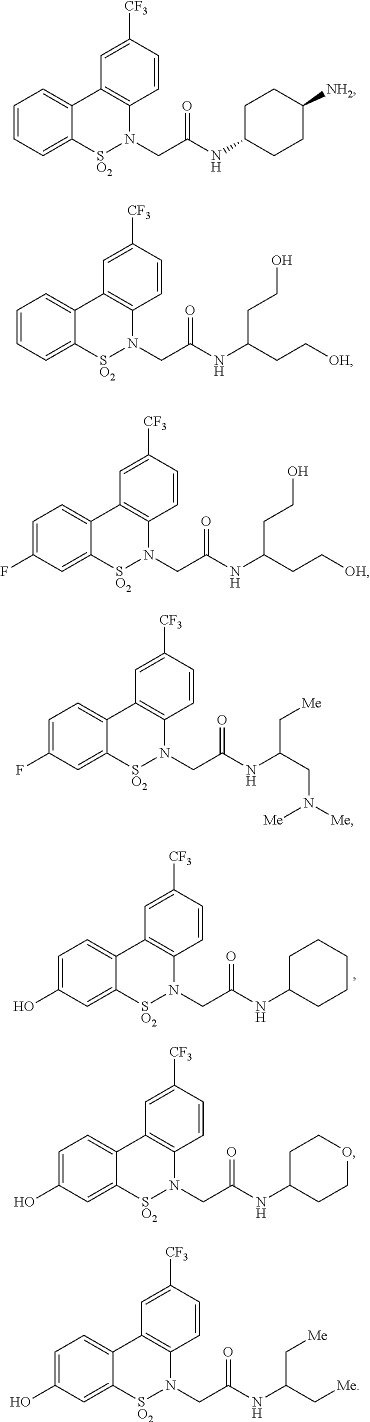

It is a further object of the invention a compound of general Formula (III):

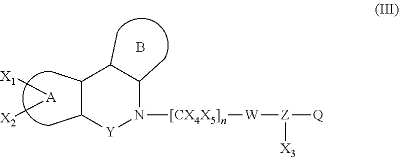

wherein
A is a benzene ring or a five- or six heteroaromatic ring;
B is a benzene ring of general structure:

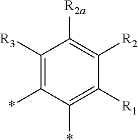

wherein:
R₁, R₂ and R₃ are each independently selected from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₆alkyl, OC₁₋₄alkylamino, hydroxy, SC₁₋₄alkyl;
R₂ₐ is hydrogen, CF₃, F, OH, OC₁₋₄alkyl, SC₁₋₄alkyl, OC₁₋₄alkylamino with the proviso that:
   if R₂ₐ is hydrogen or F, then R₂ and/or R₃ are each independently selected from F, Cl, Br, CF₃, OMe, OH; or
   if R₁ is halogen, then R₂ₐ is hydrogen and R₂ and R₃ are each independently selected from hydrogen, halogen, nitro, cyano, thiol, C₁₋₄alkyl, haloalkyl, O-haloalkyl, OC₁. 4alkyl, NHC₁₋₄alkyl, C(═O)C₁₋₆alkyl, C(═O)OC₁₋₆alkyl, C(═O)NHC₁₋₆alkyl, OC₁₋₄ alkylamino, hydroxy, SC₁₋₄alkyl;
or B is a five- or six membered heteroaromatic ring optionally substituted by one or more substituents each independently selected from hydrogen, halogen, nitro, cyano, thiol, C₁₋₄ alkyl, haloalkyl, O-haloalkyl, OC₁₋₄alkyl, NHC₁₋₄alkyl, C(═O)C₁₋₄alkyl, C(═O)OC₁₋₆alkyl, C(═O)NHC₁₋₄alkyl, hydroxy, SC₁₋₄alkyl, OC₁₋₄alkylamino;
W is C(═O);
Y is selected from CH₂, SO₂, SO, S, C(═O), PO₂, and NR₄; preferably Y is selected from CH₂, SO₂, SO, C(═O), and NR₄;
Z is N or CH;
X₁ and X₂ are each independently selected in each instance from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino, OH, pyrrolidine, piperidine, morpholine, piperazine, N-methylpiperazine;
X₃ is hydrogen, methyl, ethyl, isopropyl or benzyl;
X₄ and X₅ are independently selected in each instance from hydrogen, C₁₋₃alkyl, haloalkyl, halogen, cycloalkyl, amino, hydroxy, cyano, nitro; n is 0, 1, 2, 3, 4;
or residues X₃ and X₄ taken together represent a single bond or a C₁₋₄alkanediyl, said single bond or said C₁₋₄alkanediyl forming together with the bridging atoms to which they are respectively linked a 5 or 6 membered heterocyclic ring;
R₄ is selected from hydrogen, C₁₋₄alkyl, C₁₋₄aminoalkyl, C₁₋₄hydroxyalkyl, C₁₋₄nitroalkyl, C₁₋₄thioalkyl, C₁₋₆haloalkyl;
Q is selected from C₁₋₈alkyl, C₁₋₈alkenyl, cycloalkyl, heterocycloalkyl, aryl ring, heteroaromatic ring, wherein:
   the C₁₋₈alkyls is optionally substituted with hydroxy, OC₁₋₄alkyl, NHC₁₋₄alkyl, N(C₁₋₄alkyl)₂, NH(C═O)C₁₋₄alkyl, aryl, heteroaryl, heterocycloalkyl, cycloalkyl, cycloalkenyl, each of said aryl, heteroaryl, heterocycloalkyl, cycloalkyl, cycloalkenyl being optionally substituted with methyl, halogen, hydroxy;
   the cycloalkyl and the heterocycloalkyl are each optionally substituted with OH, OSO₂R₅, C₁₋₃alkyl, NR₆R₇, wherein:
      R₅ is selected from hydrogen, phenyl, heteroaryl, aminophenyl and nitrophenyl; and wherein
      R₆ and R₇ are each independently selected from H, methyl, C(═O)CH₃, SO₂CH₃;
   the aryl ring or the heteroaromatic ring are each optionally substituted with one or more substituents selected from halogen, nitro, cyano, thiol, C₁₋₄alkyl, haloalkyl, O-haloalkyl, OC₁₋₄alkyl, NH₂, NHSO₂C₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino;
      and any stereoisomer, pharmaceutically acceptable salt, hydrate, solvate thereof.

Preferably, in the compound as defined above A is benzene; and/or Y is SO₂; and/or Z is N; and/or X₄ and X₅ are H.

In a preferred embodiment, the compound of formula (III) is a compound of formula (IIIA):

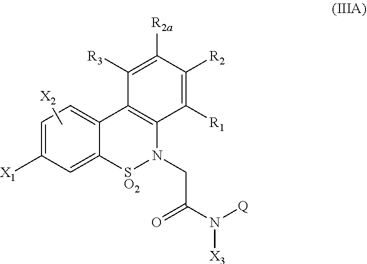

Wherein X₁, X₂, R₁, R₂, R₂ₐ, R₃ and Q are as defined above for general formula (III).

Still preferably the compound of formula (III) is selected from:

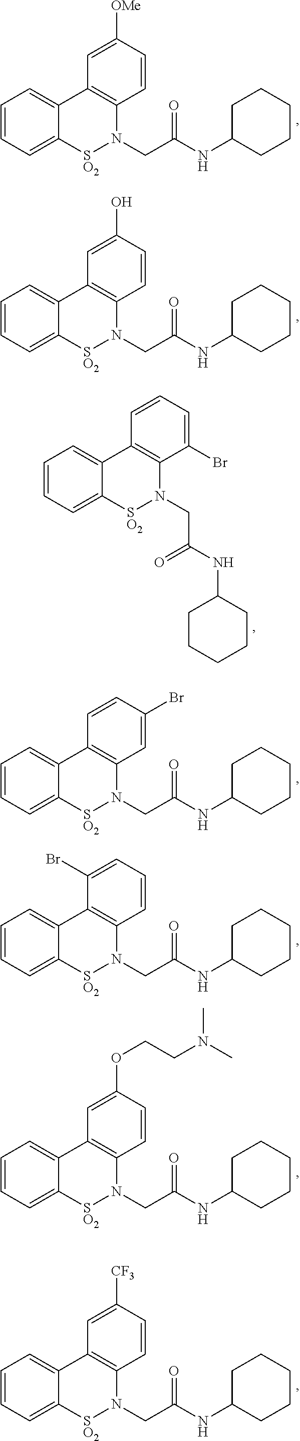

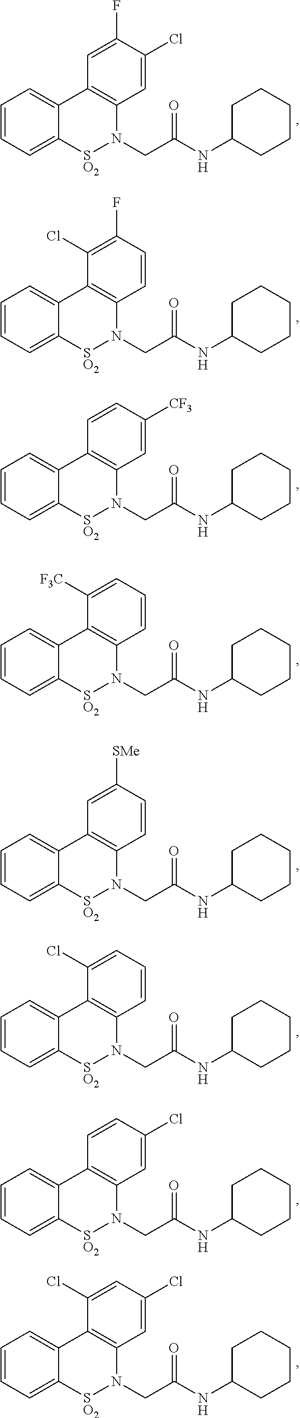

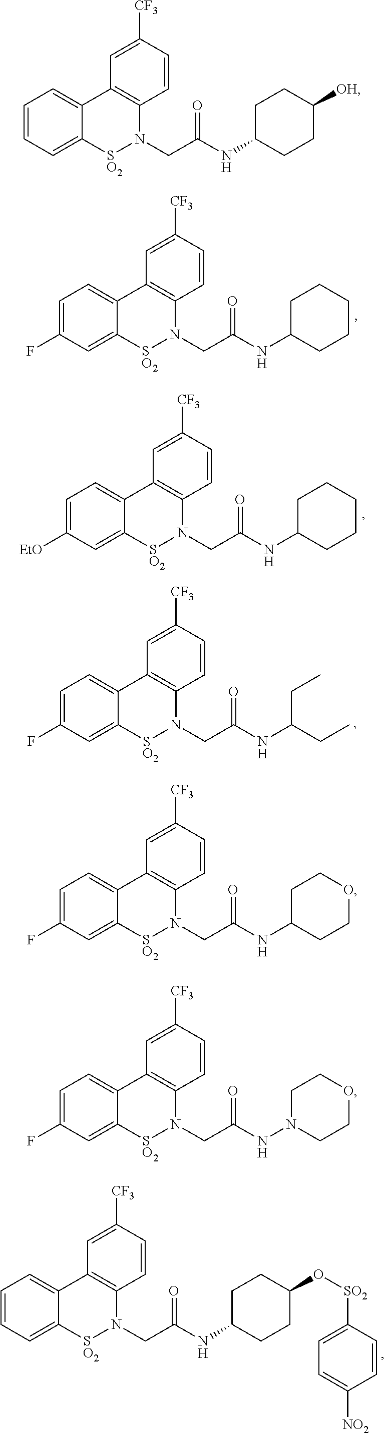

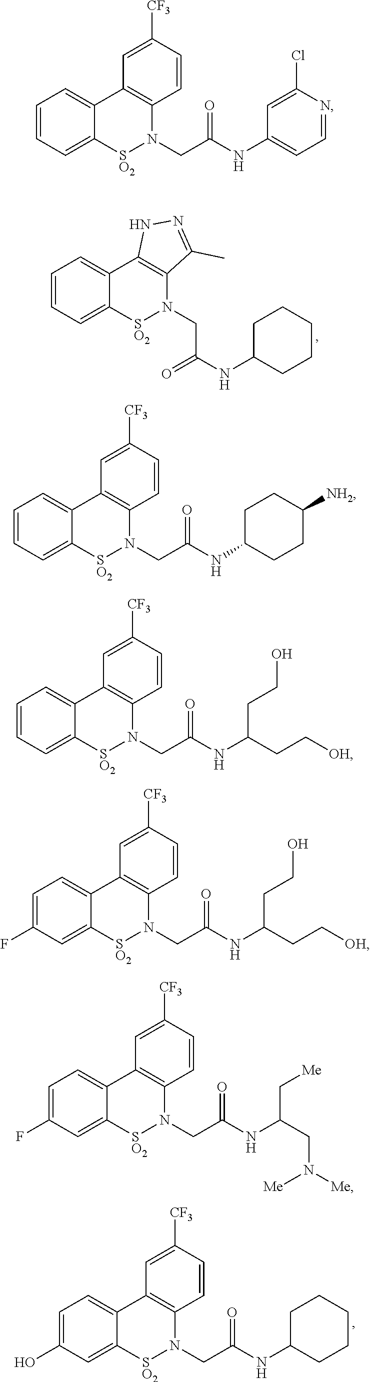

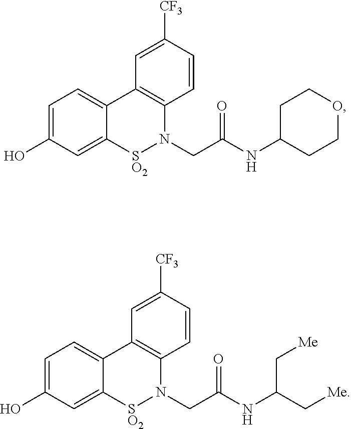 [SMILES: O=C(CN1c2ccc(C(F)(F)F)cc2-c2ccc(O)cc2S1(=O)=O)NC1CCOCC1 ; CCC(CC)NC(=O)CN1c2ccc(C(F)(F)F)cc2-c2ccc(O)cc2S1(=O)=O]

In a preferred embodiment of formula (I) or (II), B is selected from:

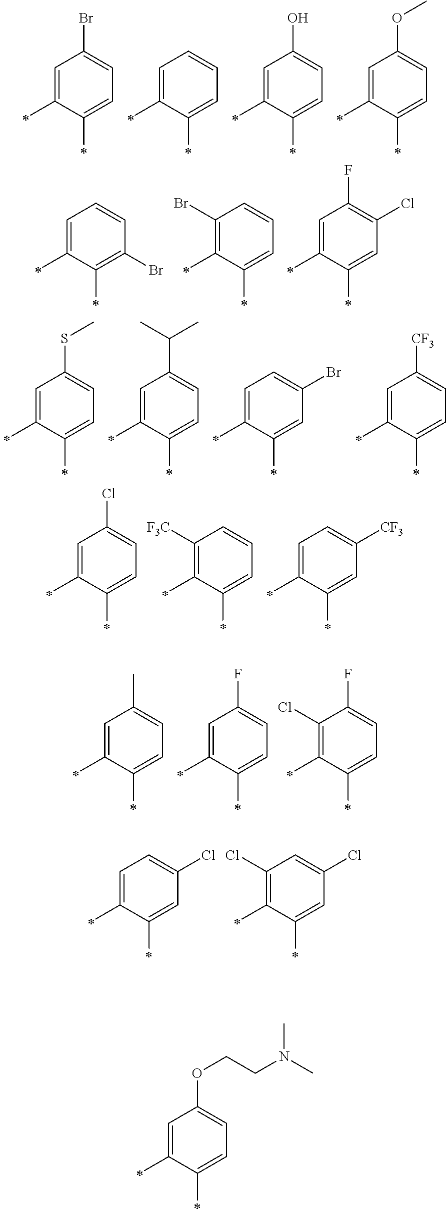

In an embodiment of formula (I), ring B is

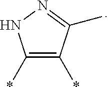 [SMILES: *c1[nH]nc(C)c1*]

In a preferred embodiment of formula (III), B is selected from:

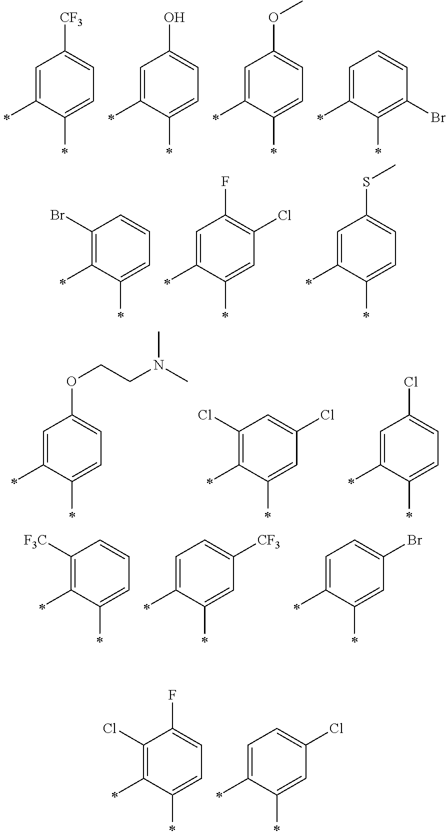

In a preferred embodiment the compound as defined above is for medical use, preferably for use in the treatment of a neurodegenerative disease or immune disease, even more preferably for use in the treatment of Prion Disease, Alzheimer Disease, Multiple Sclerosis, Autoimmune Encephalitis, Parkinson's Disease, Inflammatory Bowel Disease, Crohn's Disease.

Another aspect of the present invention relates to a method of treating a disease which benefit of modulation of the activity of PrPc, wherein said disease is Prion Disease, Alzheimer Disease, Multiple Sclerosis, Autoimmune Encephalitis, Parkinson's Disease, Inflammatory Bowel Disease, Crohn's Disease, comprising the step of administering to a patient in need thereof a therapeutically effective amount of a compound of formula (I), (II) or (III), with limitations and provisions set out above, including any pharmaceutically acceptable salt, solvate or stereoisomer thereof, as defined hereinabove.

It is a further object of the invention a pharmaceutical composition comprising at least one compound as above defined, alone or in combination with at least one further active compound, and at least one pharmaceutically acceptable excipient for use in the treatment of a neurodegenerative disease, preferably for use in the treatment of Alzheimer Disease, Prion Disease, Multiple Sclerosis and Autoimmune Encephalitis, even more preferably for use in the treatment of Multiple Sclerosis and Autoimmune Encephalitis.

It is a further object of the invention a process for the synthesis of a compound of general formula (III), wherein A is benzene and Y is SO₂, comprising the following steps:
a) reacting a compound of formula 1a with an aromatic or heteroaromatic amine of formula 1b, in the presence of a solvent like dichlorometane and an amine like pyridine, trimethylamine, diethylisopropylamine and the like, to give a compound of formula 2a:

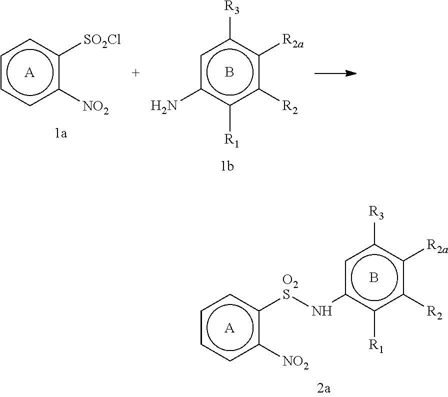

b) reducing the nitro group of compounds of formula 2a to an amino group by hydrogenation in the presence of Raney-Nichel catalyst or with SnCl₂·2H₂O under appropriate conditions, to obtain a compound of formula 3a:

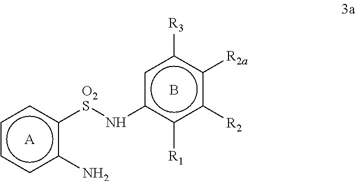

c) converting compound 3a into a compound of formula 5a

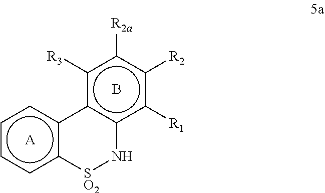

by a first step comprising reaction with NaNO₂, NaOH and HCl under appropriate conditions, and a second step employing Cu powder and DMSO as solvent at room temperature;
d) converting compound of formula 5a into a compound of formula (I):
wherein the reaction comprises at least one of the following step:
   reaction of 5a with an alkylating agent of formula hal-(CH₂)ₙ—C(═O)OEt or with an alkylating agent of formula hal-(CH₂)ₙ-Q wherein hal is bromine or chlorine;
   treatment with an amine of formula Q-NHX₃ under microwawe irradiation and neat conditions;
   coupling with an amine of formula Q-NHX₃ in the presence of a condensing agents such as TBTU in CH₂Cl₂ and DIPEA or using SOCl₂ as chlorinating agent.

As used herein, “alkyl” is intended to include both branched and straight-chain saturated aliphatic hydrocarbon groups having the specified number of carbon atoms. For example, “C₁₋₆alkyl” is defined to include groups having 1, 2, 3, 4, 5 or 6 carbons in a linear or branched arrangement and specifically includes methyl, ethyl, n-propyl, i-propyl, n-butyl, t-butyl, i-butyl, pentyl, hexyl, and so on. Preferably, “C₁₋₆alkyl” refer to “C₁₋₄alkyl” or “C₁-3alkyl”. More preferably, “C₁₋₆alkyl” or “C₁₋₃alkyl” refer to methyl.

As used herein, “C₁₋₄alkanediyl” includes methylene, 1,2-ethanediyl and the higher homologues thereof.

As used herein, “O-alkyl” or “alkoxy” represents an alkyl group of indicated number of carbon atoms attached through an oxygen bridge. “O-alkyl” therefore encompasses the definitions of alkyl above. Preferably, O-alkyl refers to a linear or branched OC₁₋₆alkyl group, OC₁₋₄alkyl group, OC₁₋₃alkyl group, or OC₁₋₂alkyl group, or OCH₃. Examples of suitable O-alkyl groups include, but are not limited to methoxy, ethoxy, n-propoxy, i-propoxy, n-butoxy, s-butoxy or t-butoxy. Preferred alkoxy groups include methoxy, ethoxy and t-butoxy.

As used herein, the terms “haloalkyl” and “O-haloalkyl” mean an alkyl or an alkoxy group in which one or more (in particular, 1 to 3) hydrogen atoms have been replaced by halogen atoms, especially fluorine or chlorine atoms. An haloalkoxy group is preferably a linear or branched haloalkoxy, more preferably a haloC₁₋₃alkoxy group, still more preferably a haloC₁₋₂alkoxy group, for example OCF₃, OCHF₂, OCH₂F, OCH₂CH₂F, OCH₂CHF₂ or OCH₂CF₃, and most especially OCF₃ or OCHF₂. An haloalkyl group is preferably a linear or branched haloalkyl group, more preferably a haloC₁₋₃alkyl group, still preferably a haloC₁₋₂alkyl group, for example, CF₃, CHF₂, CH₂F, CH₂CH₂F, CH₂CHF₂, CH₂CF₃ or CH(CH₃)CF₃. Still preferably, any one of haloalkyl, haloC₁₋₆alkyl, haloC₁₋₄alkyl group, haloC₁₋₃alkyl group refers to: CF₃, CHF₂, CH(CH₃)CF₃, CH₂CF₃ or (CH₃)₂CF₃.

As used herein the term “alkylamino” represents an alkyl group of indicated number of carbon atoms substituted by at least one amino group, wherein said amino group is —NH₂ or is further substituted with one or two alkyl groups. For example, C₁₋₄alkylamino indicates butylamine, isobutylamine, tert-butylamine, butyl-NH(CH₃), isobutyl-NH(CH₃), tert-butyl-NH(CH₃), butyl-N(CH₃)₂, isobutyl-N(CH₃)₂, tert-butyl-N(CH₃)₂, butyl-NH(C₂H₅), isobutyl-NH(C₂H₅), tert-butyl-NH(C₂H₅), butyl-N(C₂H₅)₂, isobutyl-N(C₂H₅)₂, tert-butyl-N(C₂H₅)₂, butyl-N(C₂H₅)(CH₃), isobutyl-N(C₂H₅)(CH₃), tert-butyl-N(C₂H₅)(CH₃), and the like. As used herein “OC₁₋₄alkylamino” represents the above C₁₋₄alkylamino attached through an oxygen bridge.

As used herein, “NH-alkyl” represents an alkyl group of indicated number of carbon atoms attached through a NH bridge. Preferably, NH-alkyl refers to a linear or branched NHC₁₋₆alkyl group, NHC₁₋₄alkyl group, NHC₁₋₃alkyl group, or NHC₁₋₂alkyl group, or NHCH₃. Similarly, “N(alkyl)₂” represents two alkyl groups of indicated number of carbon atoms attached through a nitrogen bridge.

As used herein, “S-alkyl” represents an alkyl group of indicated number of carbon atoms attached through a sulphur bridge. “S-alkyl” therefore encompasses the definitions of alkyl above. Preferably, S-alkyl refers to a linear or branched SC₁₋₆alkyl group, SC₁₋₄alkyl group, SC₁₋₃alkyl group, or SC₁₋₂alkyl group, or SCH₃. Examples of suitable S-alkyl groups include, but are not limited to thiomethyl, thioethyl, thiopropyl, thio-i-propyl, thio-n-butyl, thio-s-butyl or thio-t-butyl. Preferred S-alkyl groups include thiomethyl, thioethyl and thiopropyl.

As used herein, the term “aryl” means a monocyclic or polycyclic aromatic ring comprising carbon atoms and hydrogen atoms. If indicated, such aromatic ring may include one or more heteroatoms, then also referred to as “heteroaryl” or “heteroaromatic ring”. Illustrative examples of heteroaryl groups according to the invention include 5 or 6 membered heteroaryl such as thiophene, oxazole, oxadiazole, thiazole, thiadiazole, imidazole, pyrazole, pyrimidine, pyrazine and pyridine. A preferred aryl according to the present invention is phenyl. A preferred heteroaryl according to the present invention is pyridyl. Further preferred 5 membered heteroaryl rings are oxadiazole and oxazole. Said oxadiazole is preferably substituted with one methyl group.

As used herein, the term “cycloalkyl” means saturated cyclic hydrocarbon (cycloalkyl) with 3, 4, 5 or more carbon atoms and is generic to cyclopropyl, cyclobutyl, cyclopentyl, cyclohexyl and so on. The term “cycloalkyl” further refers to polycyclic saturated ring systems, such as decahydronaphthalene, octahydro-1H-indene, adamantane and the like. Said saturated ring optionally contains one or more heteroatoms (also referred to as “heterocyclyl” or “heterocyclic ring” or “heterocycloalkyl”), such that at least one carbon atom is replaced by a heteroatom selected from N, O and S, in particular from N and O. Preferably, said cycloalkyl is cyclohexyl, still preferably cyclopentyl. Preferably, said heterocycloalkyl is pyperidine, pyrrolidine, morpholine, piperazine and other cyclic amines. Still preferably said heterocycloalkyl is tetrahydrofurane or tetrahydropyrane. As used herein, the term “halogen” refers to fluorine, chlorine, bromine and iodine, of which fluorine, chlorine and bromine are preferred.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. Compounds described in this invention containing olefinic double bonds include E and Z geometric isomers, unless stated otherwise. Also included in this invention are all salt forms, polymorphs, hydrates and solvates.

The term “polymorphs” refers to the various crystalline structures of the compounds of the present invention. This may include, but is not limited to, crystal morphologies (and amorphous materials) and all crystal lattice forms. Salts of the present invention can be crystalline and may exist as more than one polymorph.

Solvates, hydrates as well as anhydrous forms of the salt or the free compound are also encompassed by the invention. The solvent included in the solvates is not particularly limited and can be any pharmaceutically acceptable solvent. Examples include water and C₁₋₄ alcohols (such as methanol or ethanol).

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18th ed., Mack Publishing Company, Easton, PA, 1990, p. 1445, the disclosure of which is hereby incorporated by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The compounds of the present invention find use in a variety of applications for human and animal health. The compounds of the present invention are small molecules capable of modulating or abrogating mutant PrPC activity. More specifically, the compounds of the invention suppress the spontaneous cytotoxicity of a mutant form of PrP (Δ105-125). Further to that, as the mutant PrP molecules sensitize cells to the cytotoxic effect of certain antibiotics, including G418 and Zeocin, the suppression of this antibiotic hypersensitivity phenotype was used as a cellular read-out for screening small molecules libraries in the DBCA assay. Using this approach, compounds of the invention have been found to display at least 30% of activity with respect to a reference compound in suppressing the neurodegenerative phenotype, preferably more than 60%, even more preferably more than 100% of the reference compound.

Compounds of the invention might potentially modulate in an indirect manner the Farnesoid X receptor (FXR)-mediated signaling pathway. Farnesoid X receptor (FXR) is a nuclear receptor for bile acids. Ligand activated-FXR regulates transcription of genes to allow feedback control of bile acid synthesis and secretion. Under physiological conditions, activation of FXR is the major mechanism to suppress bile-acid synthesis by directly inducing target genes in both the liver and intestine, including small heterodimer partner (SHP/Shp, encoded by the NROB2/Nr0b2 gene) and fibroblast growth factor (Fgf) 15 (FGF19 in humans), which in turn inhibits, or activates signaling pathways to inhibit, CYP7A1/Cyp7a1 and CYP8B1/Cyp8b1 gene transcription.³⁶ Within the present invention it has been also discovered that similarly to SM231, the FXR agonist WAY-362450 potently rescues mutant PrP toxicity. In addition, SM231 promoted significant FXR transcriptional activity in mouse primary hepatocytes, although SM derivatives do not act as direct FXR receptor agonists (data not shown). These findings, open the hypothesis that an indirect modulation of the FXR activity is involved in the mechanism of action of the compounds of the invention.

The compounds of the invention find use in the treatment of immune diseases. As used herein, “immune disease” refers to autoimmune diseases or immune system disorders. In a preferred embodiment of the invention, immune disease refers to autoimmune colitis, Inflammatory Bowel Disease or Crohn's Disease.

The compounds of the invention can be administered orally or by parenteral administration, in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the subject to be treated. The specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### BRIEF DESCRIPTION OF THE FIGURES

Embodiments and experiments illustrating the principles of the invention will be discussed with reference to the following figures:

FIG. 1. Chemical and biological characterization of SM3. The compound SM3 (referred to as LD24 in the original study²¹), structure shown in panel (A), was previously identified in a HTS screen for its ability to suppress the ΔCR PrP-dependent hypersensitivity to cationic antibiotics in a dose-dependent fashion. (B) The drug-based cell assay (DBCA) was performed as previously described^{20,25}. Briefly, HEK293 cells stably transfected with a toxic PrP mutant carrying a deletion in the central region of the protein (Δ105-125) were seeded in 24-well plates and incubated in medium containing 500 μg/mL of Zeocin, for 48 h at 37° C. Cell death in response to co-treatment with SM3 (0.1-10 μM) was evaluated by MTT assay. Data are expressed as a percentage of untreated cells.

FIG. 2. Evaluation of potency for the SM3 derivatives. The graph shows the relative ability of each compound (tested at 1 μM) to suppress the ΔCR PrP-dependent hypersensitivity to cationic antibiotics. Data are expressed as the mean percentage relative to the parent compound SM3 (referred in the graph as LD24). Error bars reflect standard deviation. Each compound has been tested in at least three biologically independent replicates (n≥3).

FIG. 3. Dose-response analysis of selected compounds. (A) Chemical structures of the selected molecules are shown in panel. (B) A dose-response analysis using the DBCA was employed to evaluate the anti-ΔCR PrP effects of the different molecules. The graphs show a quantification of the dose-dependent, rescuing effect of each molecule. Average values were obtained from a minimum of 3 independent experiments (n≥3), and expressed as a percentage of cell viability in untreated cells. Data were fitted to a sigmoidal function using a 4-parameter logistic (4PL) non-linear regression model, allowing the estimation of the half-maximal inhibitory concentration (IC₅₀). (C) The intrinsic toxicity of each compound was evaluated in naive HEK293 cells, exposed to each molecule/concentration for 48 h at 37° C. Cytotoxicity was evaluated by MTT. Average values were obtained from a minimum of 3 independent experiments (n≥3), and expressed as a percentage of cell viability in untreated cells. Data were fitted to an inverse sigmoidal function using a 4PL non-linear regression model, allowing the estimation of the half-maximal toxicity dose (LD₅₀).

FIG. 4. SM231 rescues Δβ neurotoxicity. Primary hippocampal neurons were exposed to different concentrations of Aβ oligomers for short times (10, 20 or 60 minutes) or vehicle (VHC) control. We confirmed that the oligomers induce a quick, transient phosphorylation of the Fyn kinase (results at the 20 min time point are shown in panel A). Consistent with previous observations, this effect was prevented by co-treatment with a PrPC-directed compound TMPyP. Interestingly, co-incubation with SM231 completely abrogated Aβ effects, restoring Fyn phosphorylation to normal levels. (B) Primary hippocampal neurons were incubated for 3 hours with Aβ oligomers (3 μM) or VHC control. Consistent with previous reports, we observed a decrease of several post-synaptic markers (indicated), as evaluated by western blotting of the triton X-insoluble fractions. Importantly, co-incubation with SM231 for 20 minutes prior to incubation with Aβ oligomers significantly rescued the levels of all the post-synaptic markers. The level of a control protein (actin) was not affected by either Aβ oligomers or SM231. (*p<0.05; ** p<0.01 by Student's t test).

FIG. 5. Metabolic studies on SM231. (A) The software MetaSite suggested three regions of the molecule as the main metabolic sites (red and yellow colours in the 3D structure and bold spheres indicated by empty arrows in 2D structure) with the methylene bridge predicted as the most reactive (highlighted in red/blue sphere and indicated by a solid black arrow in the 2D structure of SM231. (B) Docking experiments against the active site of CYP450 indicated that only the cyclohexyl moiety and the C-3 (gray arrows) could be metabolized because they were accessible to the hepatic metabolic enzymes. (C) Incubation (for 4 h) of SM231 with rat liver microsomes (RLM) indicated a t1/2 of 25 min (paned C at the right) and HPLC-MS analysis of the resulting mixture suggested four metabolites (MET1-4) confirming that the cyclohexyl and the C-3, at less extent, are the main metabolic sites.

FIG. 6. DBCA-based validation of newly developed derivatives. Chemical structures for each molecule are shown inside the graphs. A dose-response analysis using the DBCA was employed to evaluate the anti-ΔCR PrP effects of the different molecules. The graphs show a quantification of the dose-dependent, rescuing effect of each molecule. Average values were obtained from a minimum of 3 independent experiments (n≥3), and expressed as a percentage of cell viability in untreated cells. Data were fitted to a sigmoidal function using a 4PL non-linear regression model, allowing the estimation of the half-maximal inhibitory concentration (IC₅₀).

FIG. 7. SM884 rescue the suppression of LTP by a prion strain. The bar graph shows the quantification of the rescue effect on LTP induced by the chronic administration of SM884 to brain slices acutely treated with a lysate of cells infected with the mouse-adapted M1000 human prion strain. Values are expressed as mean+/−SEM and calculated as percentage rescue of LTP over vehicle controls. Statistically significant differences between SM884-treated and untreated slices are calculated with student t-test: M1000 vs 884 0.03 μM+M1000 p=0.0038 (**); M1000 vs 884 0.1 μM+M1000 p=0.0031 (**). M1000 n=4; 884 0.03 μM+M1000 n=4; 884 0.1 μM+M1000 n=5.

FIG. 8. DC subsets express endogenous PrPC and DC2 treated with SM231 promote Treg cells in DC-T cell co-cultures. A. Sorted mouse DC1 and DC2 cells were treated with TMP or SM231 and then subjected to western blot analysis to evaluate PrPC expression using a specific anti-PrPC antibody. Results shown are mean±S.D. from two independent EAE experiments. *P<0.05; two-tailed Mann-Whitney test. B. CD4+ LAP+FOXP3+ cell frequency in cultures of DCs (i.e. DC1 or DC2), preconditioned with TMP or SM231 or vehicle, treated with either a specific PrPC SiRNA or an SiRNA control, cultured with naïve CD4+ T cells for 5 days. Representative results of CD4+CD25+LAP+FOXP3+ cell frequency (top right quadrants) in T: DC2 co-cultures Representative results from one experiment of three.

FIG. 9. Compounds SM888 and SM889 promote tolerogenic activity in cDC2. cDC2 treated overnight with different concentrations of PrPC modulating molecules or vehicle as control, were co-cultured with CFSE-labeled CD4+ T cells, from the spleen of OT.II mice, in the presence of different concentrations of OVA. After 3 days, proliferation was analyzed by FACS to evaluate the % of T cell proliferation in response to the specific antigen. Data are shown as mean±S.D. **P<0.01, ***P<0.001, ****P<0.0001, ANOVA followed by Bonferroni multiple comparison test.

FIG. 10. Administration of the reference PrPC binding molecule TMP or of a PrPC activating molecule SM231 ameliorate EAE severity. A. Scheme of EAE induction and treatment. B EAE clinical scores (±SEM) of) mice on a C57BL/6 background, treated with different doses of SM 231 (*P<0.05; **P<0.01; ***P<0.001; Student's ttest). C. Representative staining of spinal cord sections from MOG 35-55-immunized mice treated with PBS or SM231, visualizing immune infiltrates and demyelinization at day 25 post immunization in mice treated with PBS or SM231.

FIG. 11. SM231 does not act directly on PrPC. (A) SM231 does not alter the cell-surface localization of PrPC. HEK293 cells stably expressing an EGFP-tagged version of PrPC were grown to ˜60% confluence on glass coverslips, and then treated with the indicated concentrations of SM231 or CPZ for 24 h. After fixation and washing, the intrinsic green signal of EGFP-PrPC was acquired with an inverted microscope coupled with a high-resolution camera equipped with a 488 nm excitation filter. (B) SM231 does not alter the expression of PrPC. HEK293 cells expressing WT PrPC were treated with SM231 at different concentrations (indicated), for 48 hours. Total PrP levels were evaluated in whole-cell lysates by Western blot, using anti-PrP antibody D18. The picture in the upper panel illustrates a representative western blotting. Graph in the bottom panel shows the quantification of PrP levels, obtained by densitometric analysis of four independent (n=4) experiments, normalizing each value on the corresponding Ponceau S-stained lane. Bars represent the mean (±SEM), expressed as percentage of the levels in untreated cells. (C) SM231 does not bind to PrPC. The interaction of the porphyrin Fe(III)-TMPyP (abbreviated TP), chlorpromazine (CPZ) or SM231 with recombinant PrPC was evaluated by DMR. Different concentrations of each compound (0.1-1000 μM) were added to label-free microplate well surfaces (EnSpire-LFB HS microplate, Perkin Elmer) on which full-length human recombinant PrPC or BSA had previously been immobilized. Measurements were performed before (baseline) and after (final) adding the compound. The response (pm) was obtained subtracting the baseline output to the final output signals. The output signal for each well was obtained by subtracting the signal of the protein-coated reference area to the signal of uncoated area. Signals for TP (blue dots) or CPZ (green dots) were fitted (blue and green lines) to a sigmoidal function using a 4PL non-linear regression model; R²=0.99; p=0.00061. Conversely, no binding was detected for SM231, suggesting that this compound does not exert its effects by directly binding to PrPC.

FIG. 12. An FXR agonist potently rescues mutant PrP toxicity. The DBCA was employed to evaluate the ability of the two FXR agonists, tested at different concentrations (indicated), to rescue the Zeocin hypersensitivity conferred by the expression of mouse ΔCR PrP molecules expressed in HEK293 cells. Bar graphs illustrate the quantification of the dose-dependent rescuing effect of each molecule. Mean values were obtained from a minimum of 3 independent cell culture preparations, and expressed as percentage of cell viability rescue, using the following equation: R=(T−Z)/(U−Z) (R: rescuing effect; T: cell viability in compound-treated samples; Z: cell viability in zeocin-treated samples; U: cell viability in untreated samples).

FIG. 13. FXR transcriptional activity in hepatocytes treated with SM231. Murine hepatocytes were treated for 4 or 12 h, as indicated. FXR and Nr0b2 mRNA levels were assessed by RT-qPCR. mRNA levels in untreated cells were arbitrarily set to 1.

### MATERIALS AND METHODS

### Chemistry: Methods for Making the Compounds of General Formula (I)

As used herein, the following abbreviations have the following meanings. If an abbreviation is not defined, it has its generally accepted meaning.

### Abbreviations

BOP: N-(Benzenesulfonyl)-L-prolyl-L-O-(1-pyrrolidinylcarbonyl)tyrosine sodium salt; DIPEA: N,N-Diisopropylethylamine; DMF: N,N-Dimethylformamide; DMSO: N,N-dimethylsulfoxide; EtOAc: ethyl acetate; MeOH: methanol; TBTU: 2-(1H-Benzotriazole-1-yl)-1,1,3,3-tetramethylaminium tetrafluoroborate; Pyr: Pyridine;

Unless otherwise indicated, reagents and solvents were purchased from common commercial suppliers and were used as such. HPLC-grade solvents used for IPLC analysis were purchased by Sigma-Aldrich and all the employed mobile phases were degassed with 10 min sonication before use. Organic solutions were dried over anhydrous Na₂SO₄ and concentrated with a rotary evaporator at low pressure. All reactions were routinely checked by thin-layer chromatography (TLC) on silica gel 60F254 (Merck) and visualized by using UV or iodine. Microwave assisted reactions were carried out using the microwave reactor Biotage Initiator 2.0 and parameters were adjusted according to the reaction as indicated in the following examples. Flash chromatography on Merck silica gel 60 (mesh 230-400). Melting points were determined in capillary tubes (Büchi Electrotermal model 9100) and are uncorrected. ¹H NMR spectra were recorded at 200 or 400 MHz (Bruker Avance DRX-200 or 400, respectively) while ¹³C NMR spectra were recorded at 100 MHz (Bruker Avance DRX-400) as well as 2D ¹H NMR NOESY run in phase sensitive mode. Chemical shifts are given in ppm (δ) relative to TMS. Spectra were acquired at 298 K. Data processing was performed with standard Bruker software XwinNMR and the spectral data are consistent with the assigned structures. Yields were of purified products and were not optimized. All compounds were ≥95% pure as determined by LC/MS using an Agilent 1290 Infinity System machine equipped with DAD detector from 190 to 640 nm. The purity was revealed at 270.44 nm using a ZORBAX Eclipse Plus C18 (2.1×50 mm, 1.8 μM particle size column) reverse phase was used with gradient of 0-100% CH₃CN with 0.1% formic acid (channel B) in water with 0.1% formic acid (channel A) for 20 min at 0.3 mL/min. Injection volume was 0.5 μL with a column temperature of 50° C. All compounds were ≥95% pure as determined by HPLC using a Waters System machine equipped with UV detector. The purity was revealed at 254 and 270 nm by using an X Terra C18 (x mm, μM particle size column) reverse phase was used with isocratic eluent 70:30 of CH₃CN (channel C) and water with 0.1% formic acid (channel B) for 10 min at 1 mL/min. Injection volume was 20 μL with a column temperature of 25° C. Peak retention time is given in minutes. HRMS Detection was based on electrospray ionization (ESI) in negative polarity using Agilent 1290 Infinity System equipped with a MS detector Agilent 6540UHD Accurate Mass Q-TOF.

The compounds of the invention can be prepared while using a series of chemical reactions well known to those skilled in the art, altogether making up the process for preparing said compounds and exemplified further. The processes described further are only meant as examples and by no means are meant to limit the scope of the present invention. In particular, the compounds of the present invention may be prepared according to the general procedure outlined in the following Schemes 1, 2, 3 and 4. Alternative synthetic pathways and analogues structures will be apparent to those skilled in the art of organic chemistry.

Scheme 1 shows a procedure useful for making heterocyclic compounds of formula (I) having a dibenzo[c,e][1,2]thiazine 5,5 dioxide scaffold, i.e. wherein A is a phenyl, B is a phenyl, Y is a SO₂ group, and wherein W is carbonyl, Z is nitrogen, X₄ and X₅ are hydrogen, n is as defined for general formula (I). The Q substituent can be selected from those described in general formula (I).

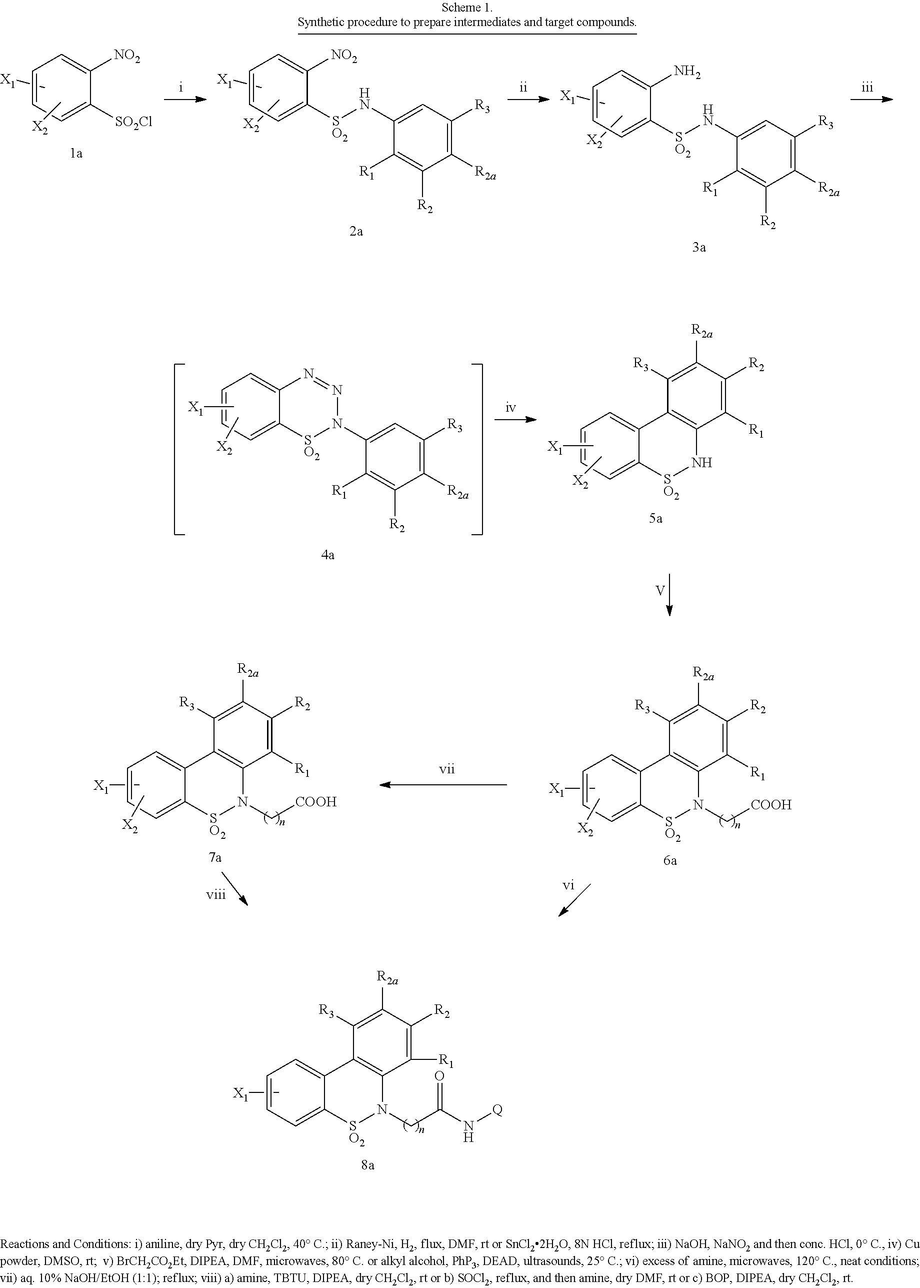

Coupling reaction of an appropriate 2-nitro-benzensulfonyl chloride of formula (1a) with unsubstituted or functionalized aniline, carried-out at 40° C. in dry pyridine, affords the corresponding aryl 2-nitrobenzensulfonamides of formula (2a), in high yields. The nitro group of intermediates of formula (2a) was reduced by using a catalytic reduction employing Raney-Ni and H₂ flux or SnCl₂·2H₂O in acidic conditions, depending on the substrates, to afford amino compounds of formula (3a) which were subsequently diazotized using NaNO₂ and HCl followed by addition of NaOH promoting in situ conversion of diazo compounds into unstable intermediates of formula (4a). These latter were immediately isolated as crude products and converted to intermediates of formula (5a) in moderate yields, employing Cu powder and DMSO as solvent at room temperature. Compounds of formula (5a) were reacted with ethyl 2-bromoacetate, under microwave irradiation at 50° C. for 15 min. in DMF and using DIPEA as scavenger to afford compounds of formula (6a) in good yields. Some intermediates of formula (5a) were alkylated exploiting a Mitsunobu reaction to give certain compounds of formula (6a). Some examples of intermediates of formula (6a) were treated with an excess of amines as defined by Q substituents, employing microwaves irradiation at 120° C. and neat conditions to give target compounds of formula (8a). In some cases, intermediates of formula (7a) were treated with a mixture of 10% aq. NaOH and EtOH (1:1 ratio) to afford the corresponding carboxylic acids of formula (7a) which were coupled with aryl amines or alkyl amines, as defined by the Q substituent, and exploiting two different methods that entails the use of condensing agents such as TBTU in CH₂Cl₂ and using DIPEA as scavenger or the use of SOCl₂ as chlorinating agent followed by the addition of amines to give other examples of target compounds of formula (8a). In some examples, di-substituted anilines were used to prepare intermediates of formula (5a) as mixture of regioisomers that were used as it is for the next reaction steps to obtain certain compounds of formula (8a); regioisomers were then separated into final compounds by flash chromatography to afford each pure regioisomer.

Scheme 2 shows a procedure useful for making heterocyclic compounds of formula (I) having a dibenzo[c,e][1,2]thiazine 5,5 dioxide scaffold wherein A is a phenyl, B is a phenyl, Y is a SO₂ group, W is absent, Z is nitrogen, X₄ and X₅ are hydrogen, n is as defined for general formula (I). The Q substituent can be selected from those described in general formula (I).

### Scheme 2. Synthetic Procedure for the Preparation of Some Target Compounds.

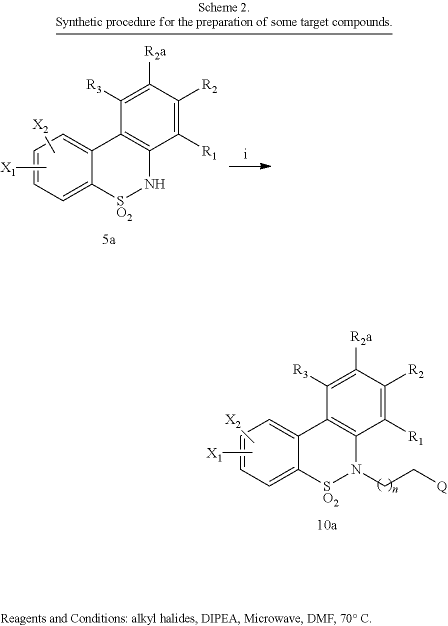

Certain compounds of formula (5a) were alkylated using bromo/chloroalkyls wherein Z was chosen between those substituents reported in formula (I) and with n=1, 2, 3, 4 by using the appropriate dihalide under microwave irradiation at 80° C. and using DIPEA as scavenger. Scheme 3 shows a procedure for synthesizing compounds SM226 and SM230 starting from a compound SM225 which was demethylated employing BBr₃ in CH₂Cl₂ and added at −60° C. The reaction was then maintained at −30° C. to give the hydroxyl derivative SM226 used as intermediate for a successive O-alkylation using (2-chloroethyl)dimethylamine hydrochloride and Cs₂CO₃ in DMF at 80° C. to give the target compound SM230.

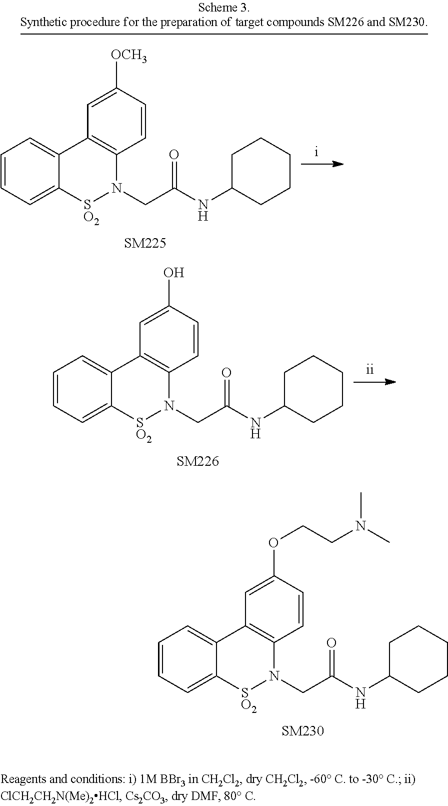 [SMILES: COc1ccc2c(c1)-c1ccccc1S(=O)(=O)N2CC(=O)NC1CCCCC1 ; O=C(CN1c2ccc(O)cc2-c2ccccc2S1(=O)=O)NC1CCCCC1 ; CN(C)CCOc1ccc2c(c1)-c1ccccc1S(=O)(=O)N2CC(=O)NC1CCCCC1]

Scheme 4 depict an example of compound of formula (I) wherein A is a phenyl, B is a 3-methyl-pyrazole, Y is a SO₂ group and W is carbonyl and the Q substituent can be selected from those indicated in the formula (I).

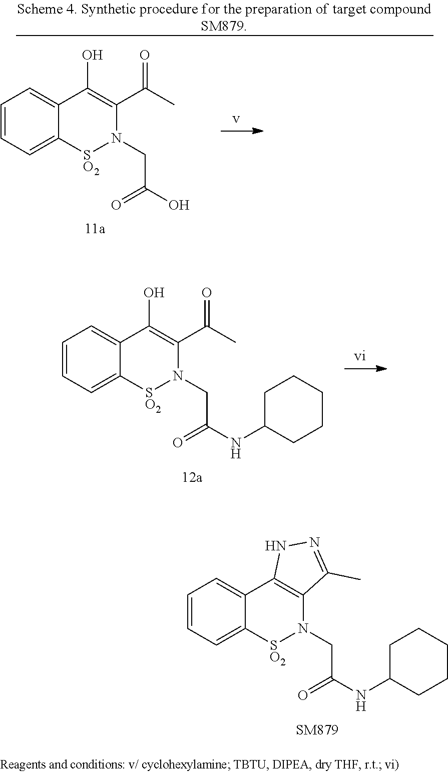 [SMILES: CC(=O)C1=C(O)c2ccccc2S(=O)(=O)N1CC(=O)O ; CC(=O)C1=C(O)c2ccccc2S(=O)(=O)N1CC(=O)NC1CCCCC1 ; Cc1n[nH]c2c1N(CC(=O)NC1CCCCC1)S(=O)(=O)c1ccccc1-2]

Compound 11a, reported in a Korean patent KR2011060653, was reacted with cyclohexylamine by using TBTU as condensing agent in presence of DIPEA affording intermediate 12a in good yield which was then condensed with hydrazine monohydrate in neat conditions at 60° C. giving the target product SM879.

The following examples are provided for the purpose of illustrating the present invention and by no means should be interpreted to limit the scope of the present invention.

TABLE 1
List of target compounds prepared in this invention - reference compound
SM3 is included

| Compound | Chemical structure of | Experimental |
|---|---|---|
| Code | compound of formula 8a | procedure Example |
| SM3 | 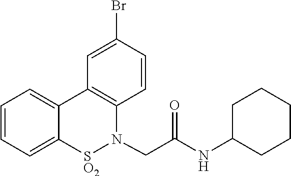 [SMILES: O=C(CN1c2ccc(Br)cc2-c2ccccc2S1(=O)=O)NC1CCCCC1] | 48 |
| SM4 | 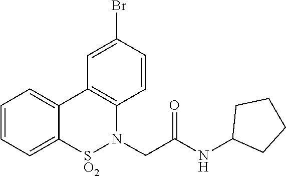 [SMILES: O=C(CN1c2ccc(Br)cc2-c2ccccc2S1(=O)=O)NC1CCCC1] | 49 |
| SM5 | 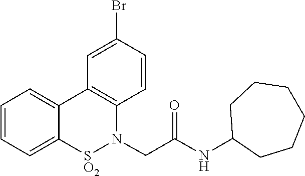 [SMILES: O=C(CN1c2ccc(Br)cc2-c2ccccc2S1(=O)=O)NC1CCCCCC1] | 50 |
| SM6 | 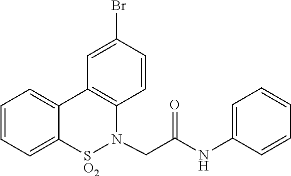 [SMILES: O=C(CN1c2ccc(Br)cc2-c2ccccc2S1(=O)=O)Nc1ccccc1] | 67 |
| SM7 | 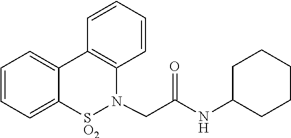 [SMILES: O=C(CN1c2ccccc2-c2ccccc2S1(=O)=O)NC1CCCCC1] | 62 |
| SM8 | 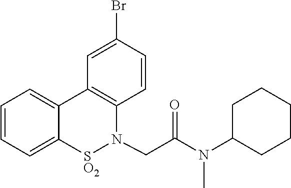 [SMILES: CN(C(=O)CN1c2ccc(Br)cc2-c2ccccc2S1(=O)=O)C1CCCCC1] | 68 |
| SM9 | 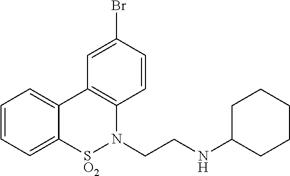 [SMILES: O=S1(=O)c2ccccc2-c2cc(Br)ccc2N1CCNC1CCCCC1] | 45 |
| SM10 | 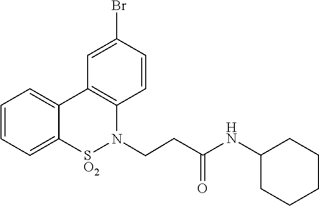 [SMILES: O=C(CCN1c2ccc(Br)cc2-c2ccccc2S1(=O)=O)NC1CCCCC1] | 51 |
| SM11 | 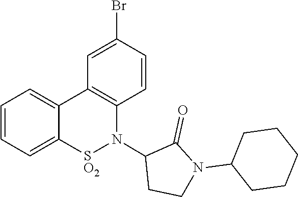 [SMILES: O=C1C(N2c3ccc(Br)cc3-c3ccccc3S2(=O)=O)CCN1C1CCCCC1] | 46 |
| SM225 | 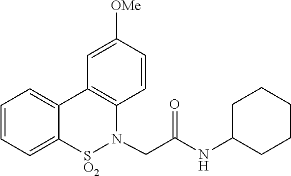 [SMILES: COc1ccc2c(c1)-c1ccccc1S(=O)(=O)N2CC(=O)NC1CCCCC1] | 55 |
| SM226 | 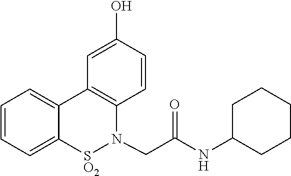 [SMILES: O=C(CN1c2ccc(O)cc2-c2ccccc2S1(=O)=O)NC1CCCCC1] | 63 |
| SM227 | 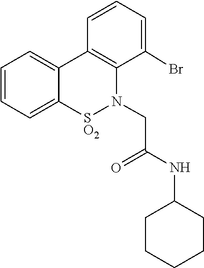 [SMILES: O=C(CN1c2c(Br)cccc2-c2ccccc2S1(=O)=O)NC1CCCCC1] | 56 |
| SM228 | 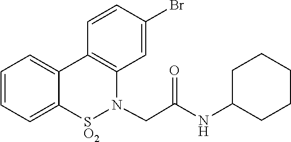 [SMILES: O=C(CN1c2cc(Br)ccc2-c2ccccc2S1(=O)=O)NC1CCCCC1] | 57 |
| SM229 | 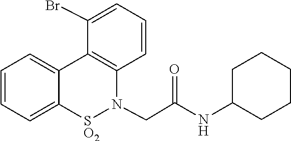 [SMILES: O=C(CN1c2cccc(Br)c2-c2ccccc2S1(=O)=O)NC1CCCCC1] | 57 |
| SM230 | 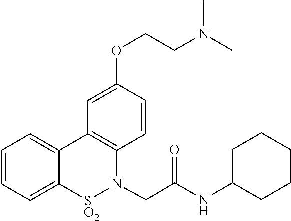 [SMILES: CN(C)CCOc1ccc2c(c1)-c1ccccc1S(=O)(=O)N2CC(=O)NC1CCCCC1] | 64 |
| SM231 | 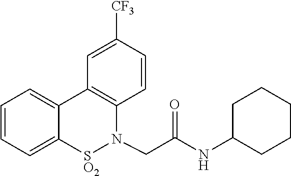 [SMILES: O=C(CN1c2ccc(C(F)(F)F)cc2-c2ccccc2S1(=O)=O)NC1CCCCC1] | 53 |
| SM254 | 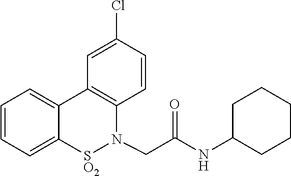 [SMILES: O=C(CN1c2ccc(Cl)cc2-c2ccccc2S1(=O)=O)NC1CCCCC1] | 52 |
| SM336 | 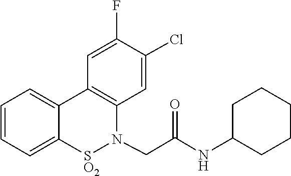 [SMILES: O=C(CN1c2cc(Cl)c(F)cc2-c2ccccc2S1(=O)=O)NC1CCCCC1] | 60 |
| SM337 | 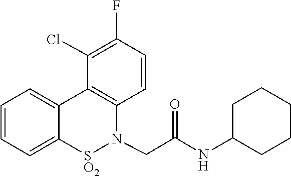 [SMILES: O=C(CN1c2ccc(F)c(Cl)c2-c2ccccc2S1(=O)=O)NC1CCCCC1] | 60 |
| SM338 | 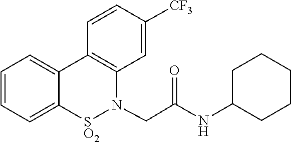 [SMILES: O=C(CN1c2cc(C(F)(F)F)ccc2-c2ccccc2S1(=O)=O)NC1CCCCC1] | 59 |
| SM339 | 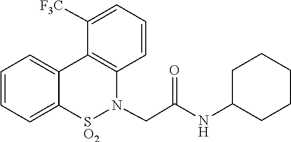 [SMILES: O=C(CN1c2cccc(C(F)(F)F)c2-c2ccccc2S1(=O)=O)NC1CCCCC1] | 59 |
| SM340 | 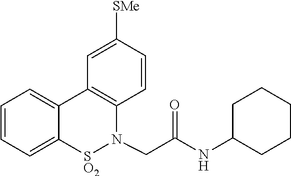 [SMILES: CSc1ccc2c(c1)-c1ccccc1S(=O)(=O)N2CC(=O)NC1CCCCC1] | 54 |
| SM585 | 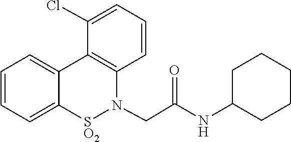 [SMILES: O=C(CN1c2cccc(Cl)c2-c2ccccc2S1(=O)=O)NC1CCCCC1] | 58 |
| SM586 | 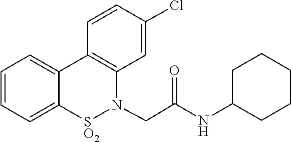 [SMILES: O=C(CN1c2cc(Cl)ccc2-c2ccccc2S1(=O)=O)NC1CCCCC1] | 58 |
| SM587 | 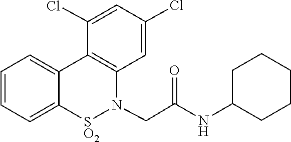 [SMILES: O=C(CN1c2cc(Cl)cc(Cl)c2-c2ccccc2S1(=O)=O)NC1CCCCC1] | 61 |
| SM588 | 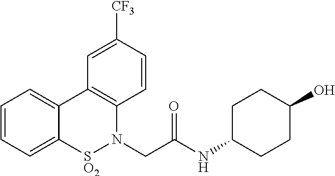 [SMILES: O=C(CN1c2ccc(C(F)(F)F)cc2-c2ccccc2S1(=O)=O)N[C@H]1CC[C@H](O)CC1] | 78 |
| SM882 | 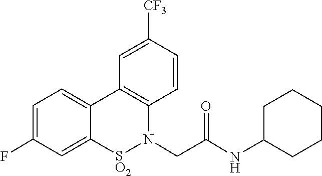 [SMILES: O=C(CN1c2ccc(C(F)(F)F)cc2-c2ccc(F)cc2S1(=O)=O)NC1CCCCC1] | 70 |
| SM883 | 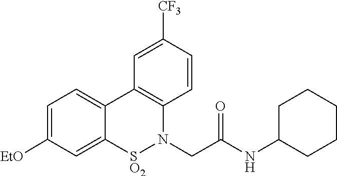 [SMILES: CCOc1ccc2c(c1)S(=O)(=O)N(CC(=O)NC1CCCCC1)c1ccc(C(F)(F)F)cc1-2] | 70 |
| SM884 | 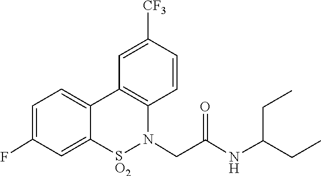 [SMILES: CCC(CC)NC(=O)CN1c2ccc(C(F)(F)F)cc2-c2ccc(F)cc2S1(=O)=O] | 71 |
| SM885 | 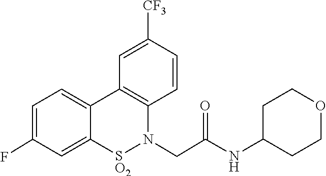 [SMILES: O=C(CN1c2ccc(C(F)(F)F)cc2-c2ccc(F)cc2S1(=O)=O)NC1CCOCC1] | 72 |
| SM881 | 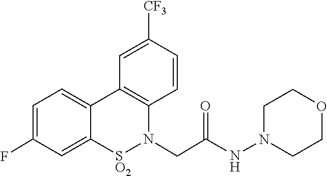 [SMILES: O=C(CN1c2ccc(C(F)(F)F)cc2-c2ccc(F)cc2S1(=O)=O)NN1CCOCC1] | 73 |
| SM589 | 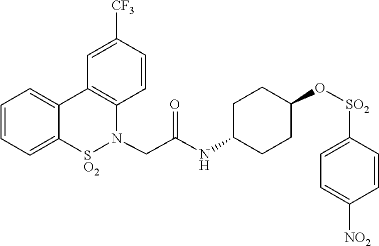 [SMILES: O=C(CN1c2ccc(C(F)(F)F)cc2-c2ccccc2S1(=O)=O)N[C@H]1CC[C@H](OS(=O)(=O)c2ccc([N+](=O)[O-])cc2)CC1] | 79 |
| SM655 | 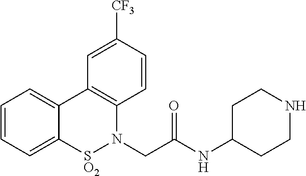 [SMILES: O=C(CN1c2ccc(C(F)(F)F)cc2-c2ccccc2S1(=O)=O)NC1CCNCC1] | 77 |
| SM656 | 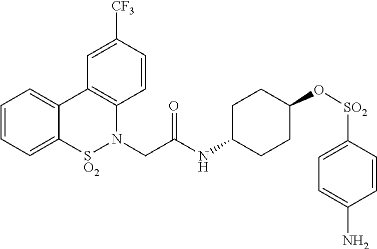 [SMILES: Nc1ccc(S(=O)(=O)O[C@H]2CC[C@H](NC(=O)CN3c4ccc(C(F)(F)F)cc4-c4ccccc4S3(=O)=O)CC2)cc1] | 80 |
| SM880 | 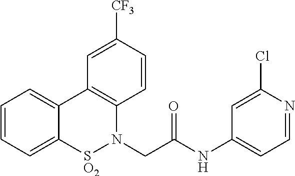 [SMILES: O=C(CN1c2ccc(C(F)(F)F)cc2-c2ccccc2S1(=O)=O)Nc1ccnc(Cl)c1] | 74 |
| SM879 | 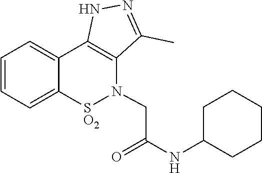 [SMILES: Cc1n[nH]c2c1N(CC(=O)NC1CCCCC1)S(=O)(=O)c1ccccc1-2] | 82 |
| SM886 | 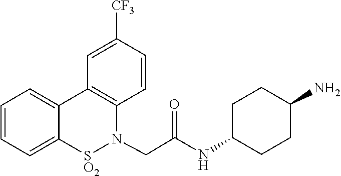 [SMILES: N[C@H]1CC[C@H](NC(=O)CN2c3ccc(C(F)(F)F)cc3-c3ccccc3S2(=O)=O)CC1] | 83 |
| SM887 | 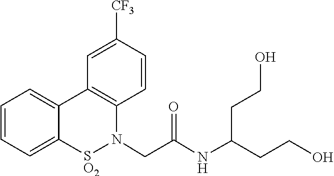 [SMILES: O=C(CN1c2ccc(C(F)(F)F)cc2-c2ccccc2S1(=O)=O)NC(CCO)CCO] | 84 |
| SM888 | 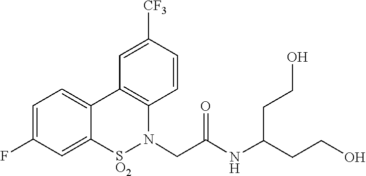 [SMILES: O=C(CN1c2ccc(C(F)(F)F)cc2-c2ccc(F)cc2S1(=O)=O)NC(CCO)CCO] | 85 |
| SM889 | 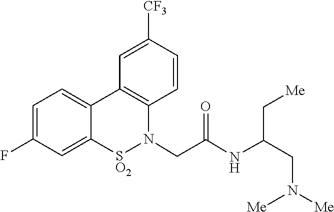 [SMILES: CCC(CN(C)C)NC(=O)CN1c2ccc(C(F)(F)F)cc2-c2ccc(F)cc2S1(=O)=O] | 86 |
| SM890 | 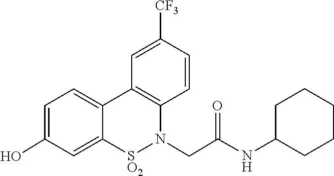 [SMILES: O=C(CN1c2ccc(C(F)(F)F)cc2-c2ccc(O)cc2S1(=O)=O)NC1CCCCC1] | 88 |
| SM891 | 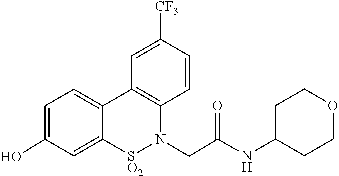 [SMILES: O=C(CN1c2ccc(C(F)(F)F)cc2-c2ccc(O)cc2S1(=O)=O)NC1CCOCC1] | 89 |
| SM892 | 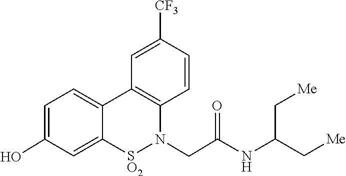 [SMILES: CCC(CC)NC(=O)CN1c2ccc(C(F)(F)F)cc2-c2ccc(O)cc2S1(=O)=O] | 90 |

The following examples are compounds purchased by AMBINTER and tested as it is for their biological activities. The synthetic procedures reported in schemes 1-3 can be easily adapted to prepare commercially available compounds whose synthesis has not been reported yet.

TABLE 2
List of target compounds of general formula 8a purchased from commercial
sources.

| | | |
|---|---|---|
| SM162 | COM129 | 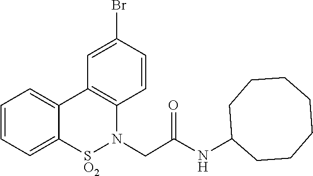 [SMILES: O=C(CN1c2ccc(Br)cc2-c2ccccc2S1(=O)=O)NC1CCCCCCC1] |
| SM163 | COM130 | 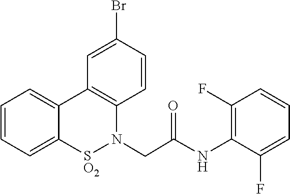 [SMILES: O=C(CN1c2ccc(Br)cc2-c2ccccc2S1(=O)=O)Nc1c(F)cccc1F] |
| SM164 | COM131 | 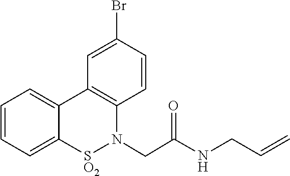 [SMILES: C=CCNC(=O)CN1c2ccc(Br)cc2-c2ccccc2S1(=O)=O] |
| SM165 | COM132 | 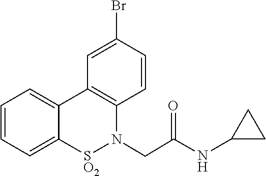 [SMILES: O=C(CN1c2ccc(Br)cc2-c2ccccc2S1(=O)=O)NC1CC1] |
| SM166 | COM133 | 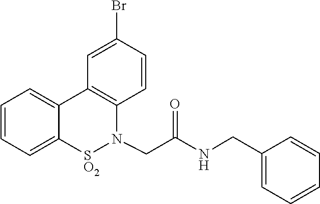 [SMILES: O=C(CN1c2ccc(Br)cc2-c2ccccc2S1(=O)=O)NCc1ccccc1] |
| SM167 | COM134 | 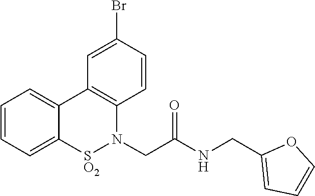 [SMILES: O=C(CN1c2ccc(Br)cc2-c2ccccc2S1(=O)=O)NCc1ccco1] |
| SM168 | COM135 | 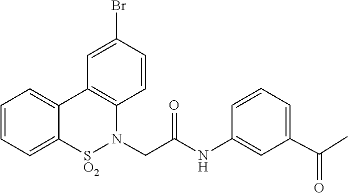 [SMILES: CC(=O)c1cccc(NC(=O)CN2c3ccc(Br)cc3-c3ccccc3S2(=O)=O)c1] |
| SM169 | COM136 | 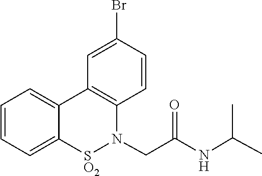 [SMILES: CC(C)NC(=O)CN1c2ccc(Br)cc2-c2ccccc2S1(=O)=O] |
| SM170 | COM137 | 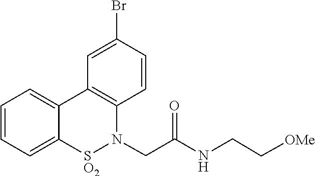 [SMILES: COCCNC(=O)CN1c2ccc(Br)cc2-c2ccccc2S1(=O)=O] |
| SM171 | COM138 | 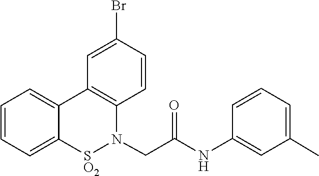 [SMILES: Cc1cccc(NC(=O)CN2c3ccc(Br)cc3-c3ccccc3S2(=O)=O)c1] |
| SM172 | COM139 | 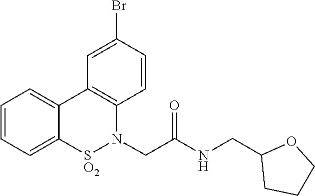 [SMILES: O=C(CN1c2ccc(Br)cc2-c2ccccc2S1(=O)=O)NCC1CCCO1] |
| SM173 | COM140 | 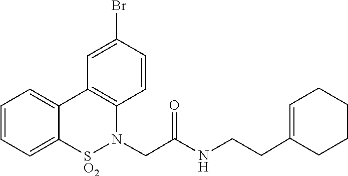 [SMILES: O=C(CN1c2ccc(Br)cc2-c2ccccc2S1(=O)=O)NCCC1=CCCCC1] |
| SM174 | COM141 | 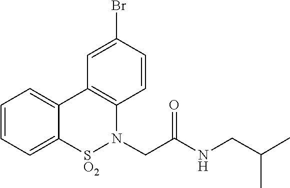 [SMILES: CC(C)CNC(=O)CN1c2ccc(Br)cc2-c2ccccc2S1(=O)=O] |
| SM175 | COM142 | 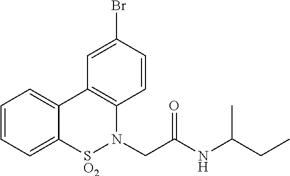 [SMILES: CCC(C)NC(=O)CN1c2ccc(Br)cc2-c2ccccc2S1(=O)=O] |
| SM176 | COM143 | 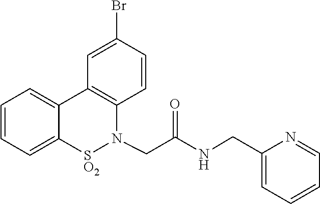 [SMILES: O=C(CN1c2ccc(Br)cc2-c2ccccc2S1(=O)=O)NCc1ccccn1] |
| SM177 | COM144 | 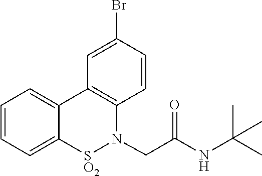 [SMILES: CC(C)(C)NC(=O)CN1c2ccc(Br)cc2-c2ccccc2S1(=O)=O] |
| SM178 | COM145 | 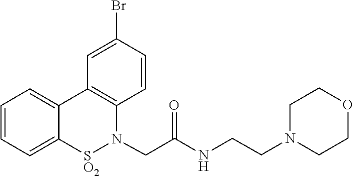 [SMILES: O=C(CN1c2ccc(Br)cc2-c2ccccc2S1(=O)=O)NCCN1CCOCC1] |
| SM179 | COM146 | 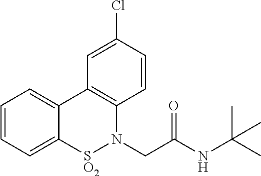 [SMILES: CC(C)(C)NC(=O)CN1c2ccc(Cl)cc2-c2ccccc2S1(=O)=O] |
| SM180 | COM147 | 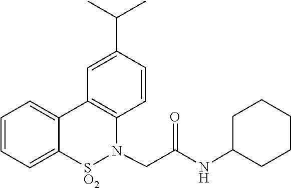 [SMILES: CC(C)c1ccc2c(c1)-c1ccccc1S(=O)(=O)N2CC(=O)NC1CCCCC1] |
| SM181 | COM148 | 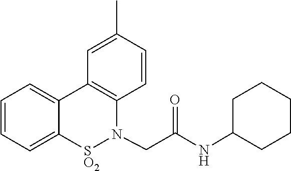 [SMILES: Cc1ccc2c(c1)-c1ccccc1S(=O)(=O)N2CC(=O)NC1CCCCC1] |
| SM219 | COM149 | 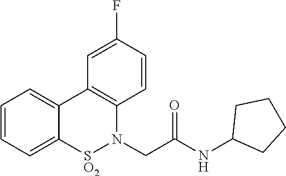 [SMILES: O=C(CN1c2ccc(F)cc2-c2ccccc2S1(=O)=O)NC1CCCC1] |
| SM220 | COM150 | 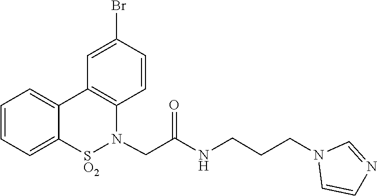 [SMILES: O=C(CN1c2ccc(Br)cc2-c2ccccc2S1(=O)=O)NCCCn1ccnc1] |
| SM221 | COM151 | 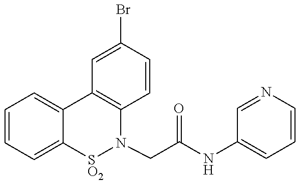 [SMILES: O=C(CN1c2ccc(Br)cc2-c2ccccc2S1(=O)=O)Nc1cccnc1] |
| SM222 | COM152 | 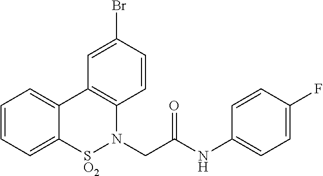 [SMILES: O=C(CN1c2ccc(Br)cc2-c2ccccc2S1(=O)=O)Nc1ccc(F)cc1] |
| SM223 | COM153 | 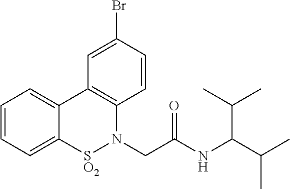 [SMILES: CC(C)C(NC(=O)CN1c2ccc(Br)cc2-c2ccccc2S1(=O)=O)C(C)C] |
| SM224 | COM154 | 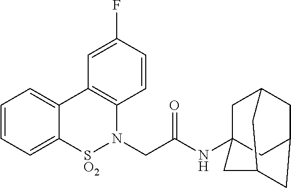 [SMILES: O=C(CN1c2ccc(F)cc2-c2ccccc2S1(=O)=O)NC12CC3CC(CC(C3)C1)C2] |

### Experimental

General procedure to obtain nitrobenzensulfonam ides of formula 2a (Scheme 1): To a solution of commercial or synthesized 2-nitrobenzenesulfonyl chloride (1 equiv.) and the appropriate aniline (2 equiv.) in CH₂Cl₂, pyridine (1 equiv.) was added at once and the mixture was maintained under magnetic stirring at 30° C. for 2 h. After concentration to one third volume, the mixture was poured into ice-water and acidified with 2N HCl (pH=3) and after digestion under magnetic stirring a precipitate was formed. After filtration the crude was then triturated with cyclohexane/EtOAc (8:2) and filtered again to give benzensulfonamides of formula 2a.

### Example 1

2-nitro-N-[3-(trifluoromethyl)phenyl]benzenesulfonamide: Following the above general procedure and using 3-trifluoromethylaniline the compound was obtained in 93% yield as red solid: mp 132.6-132.7° C.; ¹H NMR (200 MHz, acetone-d₆): δ 9.40 (brs, 1H, NH), 8.10-7.75 (m, 4H, Ar—H), 7.60-7.30 (m, 4H, Ar—H).

### Example 2

N-(3-chloro-4-fluorophenyl)-2-nitrobenzenesulfonamide: Following the above general procedure and using 3-chloro-4-fluoroaniline the compound was obtained as red solid in 90% yield: mp 110.0-110.1° C.; ¹H NMR (200 MHz, acetone-d₆): δ 9.25 (brs, 1H, NH), 8.00-7.70 (m, 4H, Ar—H), 7.50-7.40 (m, 1H, Ar—H), 7.30-7.20 (m, 2H, Ar—H).

### Example 3

N-(3,5-dichlorophenyl)-2-nitrobenzenesulfonamide: Following the general procedure above reported and using 3,4-dichloroaniline, the compound was obtained as red solid in 95% yield: mp 128.0-129.0° C.; ¹H NMR (400 MHz, acetone-d₆): δ 9.55 (brs, 1H, NH), 8.20 (d, J=1.3 and 7.8 Hz, 1H, Ar—H), 8.10-7.80 (m, 3H, Ar—H), 7.40-7.35 (m, 2H, Ar—H), 7.28 (t, J=1.8 Hz, 1H, Ar—H).

### Example 4

5-Fluoro-2-nitro-N-[4-(trifluoromethyl)phenyl]benzenesulfonamide (2a(Int-1)): Following the general procedure reported above, intermediate 5-fluoro-2-nitrobenzenesulfonyl chloride (prepared as reported in Buhr, WO 212110603) was reacted with commercial 3-trifluoromethylaniline to give the compound as red solid in 86% yield: m.p 130-132° C.; ¹H NMR (400 MHz, CDCl₃): δ 7.30-7.40 (m, 3H, Ar—H), 7.50-7.60 (m, 3H, Ar—H and NH), 7.70 (dd, J=2.8 and 7.7 Hz, 1H, Ar—H), 7.90 (dd, J=4.5 and 8.8 Hz, 1H, Ar—H).

### Example 5

N-(4-bromophenyl)-2-nitrobenzenesulfonamide: the intermediate was prepared following the procedure reported by Kurkin, A. et al. in Tetrahedron: Asymmetry, 2009, 20, 1500-1505. Melting point and spectral data are in agreement with those reported in literature.

### Example 6

N-(3-bromophenyl)-2-nitrobenzenesulfonamide: the intermediate was prepared following the procedure reported by Abramovitch, R. A. et al. in J. Org. Chem. 1977, 42, 2914-2919. Melting point and spectral data are in agreement with those reported in literature.

### Example 7

2-nitro-N-[4-(trifluoromethyl)phenyl]benzenesulfonamide: the intermediate was prepared following the procedure reported by Kang, J. G. et al. in Biosci. Biotechnol. Biochem. 2002, 66, 2677-2682. Melting point and spectral data are in agreement with those reported in literature.

### Example 8

N-[4-(methylthio)phenyl]-2-nitrobenzenesulfonamide: the intermediate was prepared following the procedure reported in PCT WO 2007/003962 A2. Melting point and spectral data are in agreement with those reported in literature.

### Example 9

Scheme 5. Synthetic Procedure for the Preparation of Intermediate of Formula 2a(Int-2).

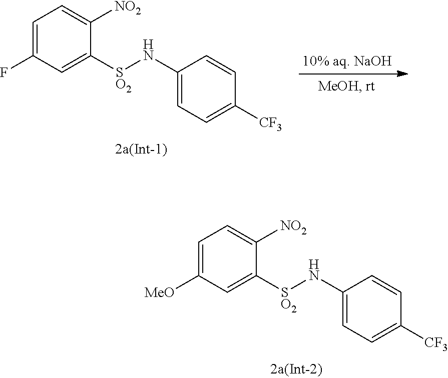 [SMILES: O=[N+]([O-])c1ccc(F)cc1S(=O)(=O)Nc1ccc(C(F)(F)F)cc1 ; COc1ccc([N+](=O)[O-])c(S(=O)(=O)Nc2ccc(C(F)(F)F)cc2)c1]

5-Methoxy-2-nitro-N-[4-(trifluoromethyl)phenyl]benzenesulfonamide (2a(Int-2)). A stirred mixture of 5-fluoro-2-nitro-N-[4-(trifluoromethyl)phenyl]benzenesulfonamide 2a(Int-1) (1.00 g, 2.86 mmol) in aqueous 10% NaOH (20 mL) and MeOH (40 mL) was kept at room temperature for 3 h. The reaction mixture was poured into ice/water and the formed precipitate was filtered off to give the title compound as a white solid in 99% yield: m.p. 142-144° C. ¹H NMR (400 MHz, CDCl₃): δ 3.89 (s, 3H, OCH₃), 7.12 (dd, J=2.7 and 8.9 Hz, 1H, H-4), 7.37 (d, J=8.4 Hz, 2H, H-2′ and H-6′), 7.48 (d, J=2.7 Hz, 1H, H-6), 7.58 (d, J=8.5 Hz, 2H, H-3′ and H-5′), 8.02 (d, J=9.0 Hz, 1H, H-3).

General procedure to obtain aminobenzensulfonamides of formula 3a (Scheme 1): A stirred solution of nitro derivative of formula (2a) (1 equiv.) in EtOH (150 mL) was hydrogenated over a catalytic amount of Raney nickel at room temperature and atmospheric pressure for 2.5 h. The mixture was then filtered over Celite and the filtrate was evaporated to dryness to give the amino derivative pure by TLC (CHCl₃/MeOH 98:2).

### Example 10

2-amino-N-[3-(trifluoromethyl)phenyl]benzenesulfonamide: Following the general procedure reported above, the compound was obtained in 96% yield as a whitish solid: mp 88.1-88.2° C. (dec.); ¹H NMR (200 MHz, acetone-d₆): δ 9.50 (bs, 1H, NH), 7.60-7.25 (m, 5H, Ar—H), 7.25-7.15 (m, 1H, Ar—H), 6.75 (d, J=8.3 Hz, 1H, Ar—H), 6.50 (t, J=8.0 Hz, 1H, Ar—H), 6.75 (bs, 2H, NH₂).

### Example 11

2-amino-N-(3-chloro-4-fluorophenyl)benzenesulfonamide: Following the procedure reported above, the compound was obtained as a grey solid, in 95% yield: mp 102.1-102.2° C.; ¹H NMR (200 MHz, acetone-d₆): δ 9.15 (bs, 1H, NH), 7.40 (dd, J=1.6 and 8.0 Hz, 1H, Ar—H), 7.25-7.00 (m, 4H, Ar—H), 6.75 (d, J=8.3 Hz, 1H, Ar—H), 6.55 (t, J=8.0 Hz, 1H, Ar—H), 5.60 (bs, 2H, NH₂).

### Example 12

2-amino-N-(3,5-dichlorophenyl)benzenesulfonamide: Following the procedure reported above, the compound was obtained as a grey solid, in 92% yield: mp 103.0-105.0° C.; ¹H NMR (400 MHz, acetone-d₆): δ 9.50 (brs, 1H, NH), 7.59 (dd, J=1.5 and 7.9 Hz, 1H, Ar—H), 7.25 (dt, J=1.5 and 7.0 Hz, 1H, Ar—H), 7.18-7.12 (m, 2H, Ar—H), 7.10 (t, J=2.4 Hz, 1H, Ar—H), 6.85 (dd, J=0.9 and 7.9 Hz, 1H, Ar—H), 6.68 (dt, J=0.9 and 7.0 Hz, 1H, Ar—H), 5.65 (brs, 2H, NH₂).

### Example 13

2-amino-N-[4-(trifluoromethyl)phenyl]benzenesulfonamide: Following the procedure reported above, the compound was obtained as a grey solid, in 85% yield (reaction time 1.5 h): mp 105.3-105.4° C.; ¹H NMR (200 MHz, DMSO-d₆): δ10.75 (bs, 1H, NH), 7.40-7.60 (m, 3H, Ar—H), 7.20-7.00 (m, 3H, Ar—H), 6.70 (d, J=8.0 Hz, 1H, Ar—H), 6.50 (t, J=8.0 Hz, 1H, Ar—H), 5.95 (bs, 2H, NH₂).

### Example 14

2-amino-N-[4-(methylthio)phenyl]benzenesulfonamide: To a stirred suspension of the corresponding nitro derivative of formula 2a (0.20 g, 0.62 mmol) in 8N HCl (9.0 mL), SnCl₂ 2H₂O (0.42 g, 1.85 mmol), dissolved in 8N HCl (2.0 mL), was added at once and the mixture was refluxed for 2 h. 10% NaOH was added to reach pH 6 and the precipitate so obtained was filtered and washed three time with CHCl₃ (3×15 mL). The fractions were collected and the solvent was dried and evaporated to dryness to obtain the amino derivative (0.10 g, 50% yield) as a crude solid used as it is in the next reaction step: mp 94.1-94.3° C.; ¹H NMR (200 MHz, CDCl₃): δ 7.40 (dd, J=1.5 and 8.0 Hz, 1H, Ar—H), 7.27-7.20 (m, 1H, Ar—H), 6.75 (d, J=8.3 Hz, 1H, Ar—H), 7.10-6.90 (m, 2H, Ar—H), 6.80-6.90 (m, 2H, Ar—H), 6.75-6.55 (m, 3H, Ar—H and NH), 4.75 (bs, 2H, NH₂).

### Example 15

2-Amino-5-fluoro-N-[4-(trifluoromethyl)phenyl]benzenesulfonamide: Following the procedure reported above, the compound was obtained as pale orange solid in 87% yield (reaction time 1 h, purification method: trituration by cyclohexane): m.p 115-117° C.; ¹H NMR (400 MHz, CDCl₃): δ 4.60 (bs, 2H, NH₂), 6.70 (dd, J=4.3 and 8.8 Hz, 1H, Ar—H), 7.05 (dt, J=2.9 and 8.7 Hz, 1H, Ar—H), 7.15 (d, J=8.4 Hz, 2H, Ar—H), 7.30 (dd, J=2.9 and 7.9 Hz, 1H, Ar—H), 7.45 (d, J=8.4 Hz, 2H, Ar—H).

### Example 16

2-amino-N-(3-bromophenyl)benzenesulfonamide: the intermediate was prepared following the procedure reported by Abramovitch, R. A. et al. in J. Org. Chem. 1977, 42, 2914-2919. Melting point and spectral data are in agreement with those reported in literature.

### Example 17

2-amino-N-(4-methoxyphenyl)benzenesulfonamide: the intermediate was prepared following the procedure reported by Ramirez-Martinez, J. F. et al. in Molecules, 2013, 18, 894-913. Spectral data are in agreement with those reported in literature.

### Example 18

2-amino-N-(4-chlorophenyl)benzenesulfonamide: the intermediate was prepared following the procedure reported by Ramirez-Martinez, J. F. et al. in Molecules, 2013, 18, 894-913. Spectral data are in agreement with those reported in literature.

### Example 19

2-amino-N-(2-bromophenyl)benzenesulfonamide: the intermediate was prepared following the procedure reported by Giannotti, D. et al. in J. Med. Chem. 1991, 34, 1356-1362. Spectral data are in agreement with those reported in literature.

### Example 20

2-amino-N-(3-chlorophenyl)benzenesulfonamide: the intermediate was prepared following the procedure reported in PCT WO 96/05185. Melting point and spectral data are in agreement with those reported in literature.

### Example 21

2-amino-N-(4-bromophenyl)benzenesulfonamide: the intermediate was prepared following the procedure reported by Ramirez-Martinez, J. F. et al. in Molecules, 2013, 18, 894-913. Spectral data are in agreement with those reported in literature.

### Example 22

2-Amino-5-methoxy-N-[4-(trifluoromethyl)phenyl]benzenesulfonamide. Following the procedure reported above, the compound was obtained as a brown solid in 73% yield (reaction time 12 h, purification method: trituration by Et₂O): m.p. 150-152° C. ¹H NMR (400 MHz, CDCl₃): δ 3.64 (s, 3H, OCH₃), 5.38 (bs, 2H, NH₂), 6.52 (d, J=8.4 Hz, 1H, H-3), 6.66-6.69 (m, 1H, H-4), 6.82 (d, J=8.0 Hz, 2H, H-2′ and H-6′), 7.15-7.21 (m, 3H, H-6, H-3′ and H-5′), 7.97 (s, 1H, NH).

General procedure to obtain 6H-dibenzo[c,e][1,2]thiazine 5,5-dioxides of formula 5a (Scheme 1): Aminobenzenesulfonamide of formula 3a (1 equiv.), NaOH (1.2 equiv.) and NaNO₂ (1.2 equiv.) were mixed in water and the obtained solution was added dropwise to HCl 37% (6 equiv.) and kept to 0° C. The muddy mixture was mixed with a glass rod for 30 min verifying the formation of diazonium salt by the β-naphtol assay. The red mixture was than diluted with H₂O and treated with AcONa powder till pH=5 and the orange solid so obtained was filtered and treated with cyclohexane to obtain instable crude solid of formula 4a. Due to its high instability, the solid was immediately added portion wise to a stirring suspension of Cu (5% of mass weight) powder in DMSO. After 30 min. the reaction mixture was filtered over Celite to remove the Cu powder and the filtrate was poured into ice/water acidifying with HCl 2N till pH=4 to afford a precipitate that was filtered under vacuum to give intermediates compounds of formula 5a.

### Example 23

9-bromo-6H-dibenzo[c,e][1,2]thiazine 5,5-dioxide: Following the general procedure reported above and starting from the corresponding amino-benzensulfonamide of formula 3a, the compound was obtained in 70% yield as brown solid and used as it is in the next reaction step: mp 229-231° C. ¹H NMR (200 MHz, DMSO-d₆):511.50 (bs, 1H, NH), 8.37 (d, J=2.3 Hz, 1H, Ar—H), 8.25 (d, J=7.6 Hz, 1H, Ar—H), 7.90 (dd, J=1.3 and 7.7 Hz, 1H, Ar—H), 7.76 (dt, J=1.4 and 7.5 Hz, 1H, Ar—H), 7.66 (dd, J=1.1 and 7.5 Hz, 1H, Ar—H), 7.59 (dd, J=2.1 and 8.6 Hz, 1H, Ar—H), 7.55-7.80 (m, 3H, H-2, H-3 and H-8), 7.10 (d, J=8.6 Hz, 1H, H-7).

### Example 24

9-chloro-6H-dibenzo[c,e][1,2]thiazine 5,5-dioxide: Following the general procedure reported above and starting from the corresponding amino-benzensulfonamide of formula 3a, the compound was obtained, after purification by flash column chromatography (CHCl₃/MeOH 98:2), as a brown solid in 26% yield (reaction time 2 h): mp 231.4-231.5° C.; ¹H NMR (200 MHz, DMSO-d₆): δ11.50 (bs, 1H, NH), 8.29-8.25 (m, 2H, Ar—H), 7.89 (dd, J=1.5 and 7.5 Hz, 1H, Ar—H), 7.75 (dt, J=1.5 and 7.5 Hz, 1H, Ar—H), 7.62 (dt, J=1.0 and 9.0 Hz, 1H, Ar—H), 7.48 (dd, J=2.3 and 7.2 Hz, 1H, Ar—H).

### Example 25

9-(Trifluoromethyl)-6H-dibenzo[c,e][1,2]thiazine 5,5-dioxide: Following the general procedure reported above and starting from the corresponding amino-benzensulfonamide of formula 3a, the compound was obtained as a brown solid in 66% yield (reaction time 1 h): mp 235-237° C. ¹H-NMR (200 MHz, CDCl₃): δ 8.22 (brs, 1H, Ar—H), 8.03-7.98 (m, 2H, Ar—H), 7.80-7.74 (m, 2H, Ar.H), 7.64-7.57 (m, 2H, Ar—H), 7.20 (brs, 1H, NH).

### Example 26

9-(Methylthio)-6H-dibenzo[c,e][1,2]thiazine 5,5-dioxide: Following the general procedure reported above and starting from the corresponding amino-benzensulfonamide of formula 3a the compound was obtained, after purification by flash column chromatography (CH₂Cl₂/MeOH 98: 2), as a pale brown solid in 25% yield (reaction time 2 h): mp 211.4-211.6° C.; ¹H-NMR (200 M Hz, DMSO-d₆): δ 11.29 (brs, 1H, NH), 8.25 (d, J=7.9 Hz, 1H, Ar—H), 8.00 (brs, 1H, Ar—H), 7.87 (d, J=7.7 Hz, 1H, Ar—H), 7.85 (t, J=7.3 Hz, 1H, Ar—H), 7.61 (t, J=7.4 Hz, 1H, Ar—H), 7.34 (d, J=8.5 Hz, 1H, Ar—H), 7.09 (d, J=8.5 Hz, 1H, Ar—H).

### Example 27

9-Methoxy-6H-dibenzo[c,e][1,2]thiazine 5,5-dioxide: Following the general procedure reported above and starting from the corresponding amino-benzensulfonamide of formula 3a the compound was obtained, after purification by flash column chromatography (CHCl₃/MeOH 97:3), as pale brown solid in 41% yield (reaction time: 30 min.): mp 198-202° C. ¹H-NMR (200 MHz, DMSO-d₆) δ 10.97 (brs, 1H, NH), 8.25 (d, J=7.7 Hz, 1H, Ar—H), 7.85 (dd, J=1.4 and 7.7 Hz, 1H, Ar—H), 7.75 (dt, J=1.4 and 7.55 Hz, 1H, Ar—H), 7.68-7.55 (m, 2H, Ar—H), 7.13-6.95 (m, 2H, Ar—H), 3.80 (s, 3H, CH₃).

### Example 28

7-Bromo-6H-dibenzo-[c,e][1,2]thiazine 5,5-dioxide: Following the general procedure reported above and starting from the corresponding amino-benzensulfonamide of formula 3a the compound was obtained as yellow solid in 78% yield: mp 188.2-188.4° C. (dec.). ¹H-NMR (400 MHz, DMSO-d₆): δ 10.80 (brs, 1H, NH), 8.25-8.30 (m, 2H, Ar—H), 7.95 (d, J=7.7 Hz, 1H, Ar—H), 7.85-7.80 (m, 2H, Ar—H), 7.72 (t, J=7.5 Hz, 1H, Ar—H), 7.36 (t, J=7.9, 1H, Ar—H).

### Example 29

8,10-Dichloro-6H-dibenzo[c,e][1,2]thiazine 5,5-dioxide: Following the general procedure reported above and starting from the corresponding amino-benzensulfonamide of formula 3a the compound was obtained as yellow solid in 77% yield: mp 190.0-191.0° C. (dec.); ¹H-NMR (200 MHz, acetone-d₆): δ10.25 (brs, 1H, NH), 8.58 (dd, J=1.7 and 7.8 Hz, 1H, Ar—H), 7.95 (dd, J=1.5 and 7.3 Hz, 1H, Ar—H), 7.83-7.65 (m, 2H, Ar—H), 7.48 (d, J=2.1 Hz, 1H, Ar—H), 7.32 (J=2.1 Hz, 1H, Ar—H).

### Example 30

3-Fluoro-9-(trifluoromethyl)-6H-dibenzo[c,e][1,2]thiazine 5,5-dioxide: Following the general procedure reported above and starting from the corresponding amino-benzensulfonamide of formula 3a the compound was obtained as pale brown solid in 83% yield: m.p. 195-197° C. ¹H NMR (400 MHz, DMSO-d₆): δ 7.35 (d, J=8.4 Hz, 1H, H-7), 7.70 (dt, J=2.7 and 8.7 Hz, 1H, H-2), 7.80 (d, J=8.5 Hz, 1H, H-8), 7.85 (dd, J=2.6 and 7.6 Hz, 1H, H-4), 8.50 (dd, J=4.8 and 8.9 Hz, 1H, H-1), 8.55 (s, 1H, H-10), 12.10 (bs, 1H, NH).

### Example 31

8-bromo-6H-dibenzo[c,e][1,2]thiazine 5,5-dioxide and 10-bromo-6H-dibenzo[c,e][1,2]thiazine 5,5-dioxide: Following the general procedure reported above and starting from the corresponding amino-benzensulfonamide of formula 3a, a mixture of two regioisomers difficult to be separated was obtained and the crude was employed without further purification for the next reaction step.

### Example 32

8-chloro-6H-dibenzo[c,e][1,2]thiazine 5,5-dioxide and 10-chloro-6H-dibenzo[c,e][1,2]thiazine 5,5-dioxide: following the general procedure reported above and starting from the corresponding amino-benzensulfonamide of formula 3a, a mixture of two regioisomers difficult to be separated was obtained and the crude was employed without further purification for the next reaction step.

### Example 33

8-(trifluoromethyl)-6H-dibenzo[c,e][1,2]thiazine 5,5-dioxide and 10-(trifluoromethyl)-6H-dibenzo[c,e][1,2]thiazine 5,5-dioxide: following the general procedure reported above and starting from the corresponding amino-benzensulfonamide of formula 3a, a mixture of two regioisomers difficult to be separated was obtained and the crude was employed without further purification for the next reaction step.

### Example 34

8-Chloro-9-fluoro-6H-dibenzo[c,e][1,2]thiazine 5,5-dioxide and 10-chloro-9-fluoro-6H-dibenzo[c,e][1,2]thiazine 5,5-dioxide: following the general procedure reported above and starting from the corresponding amino-benzensulfonamide of formula 3a, a mixture of two regioisomers difficult to be separated was obtained and the crude was employed without further purification for the next reaction step.

### Example 35

3-Methoxy-9-(trifluoromethyl)-6H-dibenzo[c,e][1,2]thiazine 5,5-dioxide: following the general procedure reported above and starting from the corresponding amino-benzensulfonamide of formula 3a, the compound was obtained in 40% yield as brown solid: m.p. 198-200° C. ¹H NMR (400 MHz, DMSO-d₆): δ 3.84 (s, 3H, OCH₃), 7.30-7.43 (m, 3H, Ar—H), 7.75 (d, J=7.2 Hz, 1H, Ar—H), 8.37 (d, J=8.6 Hz, 1H, Ar—H), 8.49 (s, 1H, Ar—H), 11.78 (s, 1H, NH).

General procedure to obtain 6H-dibenzo[c,e][1,2]thiazine 5,5-dioxides N-6 ethyl acetates of formula 6a (Scheme 1): In a microwave reactor tube, a solution of the appropriate compound of formula 5a (1 equiv.), ethyl bromoacetate (1 equiv.), and DIPEA (3 equiv.) in dry DMF (5 mL) was irradiated at 50° C. for 15 min. by setting the following experimental parameters: pressure 5 bar, cooling off, FHT on, solvent absorption very high. The pitchy mixture was poured into ice-water and extracted three times with EtOAc. The combined organic layers were washed with brine, dried, and evaporated to dryness to give a crude slurry mass that was triturated with EtOH giving a precipitate that was filtered to afford the desired compound.

### Example 36

Ethyl 2-(9-bromo-5,5-dioxido-6H-dibenzo[c,e][1,2]thiazin-6-yl)acetate: following the general procedure reported above and starting from the corresponding dibenzothiazine of formula 5a, the compound was obtained as pink solid in 75% yield: mp 89-91° C. ¹H-NMR (200 MHz, DMSO-d₆): δ 8.40 (d, J=2.3 Hz, 1H, Ar—H), 8.26 (d, J=7.6 Hz, 1H, Ar—H), 7.90-7.60 (m, 4H, Ar—H), 7.40 (d, J=8.6 Hz, 1H, Ar—H), 4.77 (s, 2H, NCH₂), 3.90 (q, J=7.0 Hz, 2H, OCH₂CH₃), 0.80 (t, J=7.0 Hz, 3H, OCH₂CH₃).

### Example 37

Ethyl (9-chloro-5,5-dioxido-6H-dibenzo[c,e][1,2]thiazin-6-yl)acetate: following the general procedure reported above and starting from the corresponding dibenzothiazine of formula 5a, the compound was obtained as brown solid in 86% yield: mp 102.8-102.9° C.; ¹H-NMR (200 MHz, CDCl₃): δ 8.47-7.99 (m, 3H, Ar—H), 7.64 (dt, J=1.5 and 7.5 Hz, 1H, Ar—H), 7.53 (dt, J=1.2 and 7.7 Hz, 1H, Ar—H), 7.34 (dd, J=2.3 and 8.7 Hz, 1H, Ar—H), 7.17 (d, J=8.7 Hz, 1H, Ar—H), 4.59 (s, 2H, NCH₂), 3.96 (q, J=7.2 Hz, 2H, OCH₂CH₃), 1.00 (t, J=7.2 Hz, 3H, OCH₂CH₃).

### Example 38

Ethyl [5,5-dioxido-9-(trifluoromethyl)-6H-dibenzo[c,e][1,2]thiazin-6-yl]acetate: following the general procedure reported above and starting from the corresponding dibenzothiazine of formula 5a, the compound was obtained as pale brown solid in 80% yield: mp 101-103° C. ¹H-NMR (200 MHz, DMSO-d₆): δ 8.60 (brs, 1H, Ar—H), 8.35 (d, J=8.0 Hz, 1H, Ar—H), 8.00-7.75 (m, 3H, Ar—H), 7.74-7.65 (m, 2H, Ar—H), 4.91 (s, 2H, NCH₂), 3.92 (q, J=7.4 Hz, 2H, OCH₂CH₃), 0.92 (t, J=7.4 Hz, 3H, OCH₂CH₃).

### Example 39

Ethyl [9-(methylthio)-5,5-dioxido-6H-dibenzo[c,e][1,2]thiazin-6-yl]acetate: following the general procedure reported above and starting from the corresponding dibenzothiazine of formula 5a, the compound was obtained as yellowish solid in 73% yield: mp 103-105° C. ¹H-NMR (200 MHz, DMSO-d₆): δ 8.02-7.91 (m, 3H, Ar—H), 7.75 (t, J=7.9 Hz, 1H, Ar—H), 7.61 (t, J=7.4 Hz, 1H, Ar—H), 7.45-7.35 (m, 1H, Ar—H), 7.26-7.23 (m, 1H, Ar—H), 4.66 (s, 2H, NCH₂), 4.05 (q, J=6.9 Hz, 2H, OCH₂CH₃), 2.57 (s, 3H, SCH₃), 1.07 (t, J=6.9 Hz, 3H, OCH₂CH₃).

### Example 40

Ethyl (9-methoxy-5,5-dioxido-6H-dibenzo[c,e][1,2]thiazin-6-yl)acetate: following the general procedure reported above and starting from the corresponding dibenzothiazine of formula 5a, the compound was obtained as brown solid in 85% yield: mp 101-104° C. ¹H-NMR (200 MHz, DMSO-d₆): δ 8.26 (d, J=7.9 Hz, 1H, Ar—H), 7.88-7.74 (m, 2H, Ar—H), 7.70-7.62 (m, 2H, Ar—H), 7.49 (d, J=8.9 Hz, 1H, Ar—H), 7.12 (dd, J=2.7 and 8.9 Hz, 1H, Ar—H), 4.75 (s, 2H, NCH₂), 3.94-3.77 (m, 5H, OCH₃ and OCH₂CH₃), 0.90 (t, J=7.0 Hz, 3H, OCH₂CH₃).

### Example 41

Ethyl (7-bromo-5,5-dioxido-6H-dibenzo[c,e][1,2]thiazin-6-yl)acetate: following the general procedure reported above and starting from the corresponding dibenzothiazine of formula 5a, the compound was obtained, after crystallization by EtOH, as pink solid in 50% yield: mp 169-171° C. ¹H-NMR (400 MHz, CDCl₃) δ 7.96-7.90 (m, 3H, Ar—H), 7.75-7.67 (m, 2H, Ar—H), 7.58 (t, J=7.8, 1H, Ar—H), 7.30 (t, J=7.9 Hz, 1H, Ar—H), 4.73 (s, 2H, NCH₂), 3.80 (q, J=7.1 Hz, 2H, OCH₂CH₃), 1.00 (t, J=7.1 Hz, 3H, OCH₂CH₃).

### Example 42

Ethyl (8,10-dichloro-5,5-dioxido-6H-dibenzo[c,e][1,2]thiazin-6-yl)acetate: following the general procedure reported above and starting from the corresponding dibenzothiazine of formula 5a, was obtained as pink solid in 90% yield: mp 172-173° C. ¹H-NMR (200 MHz, CDCl₃) δ8.49 (dd, J=1.3 and 7.9 Hz, 1H, Ar—H), 7.90 (dd, J=1.8 and 7.6 Hz, 1H, Ar—H), 7.70-7.55 (m, 2H, Ar—H), 7.10 (d, J=2.1 Hz, 1H, Ar—H), 4.51 (s, 2H, NCH₂), 4.02 (q, J=7.2 Hz, 2H, OCH₂CH₃), 1.05 (t, J=7.2 Hz, 3H, OCH₂CH₃).

### Example 43

Ethyl [3-fluoro-5,5-dioxido-9-(trifluoromethyl)-6H-dibenzo[c,e][1,2]thiazin-6-yl]acetate: following the general procedure reported above and starting from the corresponding dibenzothiazine of formula 5a, was obtained as pale brown solid in 85% yield: m.p. 175-177° C.; ¹H NMR (400 MHz, DMSO-d₆): δ1.10 (t, J=7.2 Hz, 3H, OCH₂CH₃), 4.10 (q, J=7.2 Hz, 2H, OCH₂CH₃), 4.75 (s, 1H, NCH₂), 7.35 (d, J=8.5 Hz, 1H, H-7), 7.45 (dt, J=2.7 and 8.3 Hz, 1H, H-2), 7.60-7.70 (m, 2H, H-4 and H-8), 8.00 (dd, J=4.6 and 8.8 Hz, 1H, H-1), 8.20 (s, 1H, H-10).

### Example 44

Ethyl [3-methoxy-5,5-dioxido-9-(trifluoromethyl)-6H-dibenzo[c,e][1,2]thiazin-6-yl]acetate: following the general procedure reported above and starting from the corresponding dibenzothiazine of formula 5a, the compound was obtained as pink solid in 86% yield: m.p. 190-192° C. ¹H NMR (400 MHz, DMSO-d₆): δ 1.03 (t, J=7.0 Hz, 3H, OCH₂CH₃), 3.95 (s, 3H, OCH₃), 3.98 (q, J=7.4 Hz, 2H, OCH₂CH₃), 4.93 (s, 2H, NCH₂), 7.42-7.45 (m, 2H, H-2 and H-4), 7.73 (d, J=8.6 Hz, 1H, H-7), 7.85 (d, J=8.4 Hz, 1H, H-8), 8.38 (d, J=8.4 Hz, 1H, H-1), 8.54 (s, 1H, H-10).

### Example 45

N-[2-(9-bromo-5,5-dioxido-6H-dibenzo[c,e][1,2]thiazin-6-yl)ethyl]cyclohexanamine (SM9). In a microwave reactor tube, a solution of the appropriate compound of formula 5a (0.6 g, 1.9 mmol), N-(2-chloroethyl)cyclohexanamine (0.31 g, 1.9 mmol), and DIPEA (0.66 mL, 3.8 mmol) in dry DMF (4 mL) was irradiated at 70° C. for 60 min. by setting the following experimental parameters: pressure 5 bar, cooling off, FHT on, solvent absorption very high. The residue was poured into ice-water acidified to pH 3 with 2N HCl and extracted with EtOAc (3×20 mL). The combined organic layers were washed with brine, dried, and evaporated to dryness to give a crude slurry mass that was purified by flash column chromatography eluting with CHCl₃/MeOH (97:3) giving the target compound SM9 (0.65 g, 79%) as low melting pale brown solid: ¹H NMR (400 MHz, CDCl₃): δ 8.13 (d, J=2.2 Hz, 1H, Ar—H), 7.99 (dd, J=1.1 and 7.7 Hz, 1H, H-4), 7.92 (d, J=7.2 Hz, 1H, Ar—H), 7.73 (dt, J=1.3 and 7.8 Hz, 1H, Ar—H), 7.64-7.58 (m, 2H, Ar—H), 7.40 (d, J=8.7 Hz, 1H, Ar—H), 4.00 (t, J=6.7 Hz, 2H, SO₂NCH₂), 2.85 (t, J=6.7 Hz, 2H, NCH₂), 2.40-2.25 (m, 1H, Cy-CH), 1.75-1.50 (m, 4H, Cy-CH₂), 1.25-1.00 (m, 4H, Cy-CH₂), 1.00-0.80 (m, 2H, Cy-CH₂). ¹³C NMR (100 MHz, CDCl₃): δ 137.58, 135.22, 133.05, 132.42, 131.16, 128.95, 128.45, 127.15, 125.68, 123.56, 122.51, 118.59, 56.17, 49.17, 44.52, 32.97, 25.87, 24.72. HRMS (ESI) calcd. for C₂₀H₂₃BrN₂O₂S[M⁺+H]⁺: 435.0739, found: 435.0735; LC-MS: ret. time 4.075.

### Example 46

(R,S)-3-(9-Bromo-5,5-dioxido-6H-dibenzo[c,e][1,2]thiazin-6-yl)-1-cyclohexylpyrrolidin-2-one (SM1l). Following the procedure above described for compound SM9 and using 3-bromo-1-cyclohexyl-2-pyrrolidinone³⁷, the title compound was purified by flash column chromatography, eluting with cyclohexane/EtOAc (6:4), and subsequent trituration with petroleum ether/Et₂O, to give racemic target compound SM1l as a white solid in 70% yield: mp 177-179° C. ¹H NMR (400 MHz, DMSO-d₆): δ 8.42 (d, J=2.1 Hz, 1H, Ar—H), 8.28 (d, J=7.8 Hz, 1H, Ar—H), 7.91 (d, J=7.7 Hz, 1H, Ar—H), 7.85 (t, J=8.6 Hz, 1H, Ar—H), 7.75-7.70 (m, 2H, Ar—H), 7.30 (d, J=8.6 Hz, 1H, H-7), 7.37 (d, J=8.6 Hz, 1H, Ar—H), 4.90 (t, J=9.4 Hz, 2-Pyrrolidone-CH), 3.75-3.55 (m, 1H, Cy-CH), 3.20-3.00 (m, 2H, 2-Pyrrolidone-CH), 2.10-2.00 (m, 1H, 2-Pyrrolidone-CH), 1.75-1.48 (m, 6H, Cy-CH₂ and 2-Pyrrolidone-CH), 1.40-1.00 (m, 5H, Cy-CH₂). ¹³C NMR (100 MHz, DMSO-d₆): δ 168.65, 136.83, 135.87, 133.56, 133.48, 131.05, 130.15, 129.38, 128.95, 127.40, 126.72, 122.36, 119.96, 62.56, 51.37, 25.45, 25.38, 25.33, 25.19, 22.83. HRMS (ESI) calcd for C₂₂H₂₃BrN₂O₃S: [M⁺+H]⁺: 475.0692, found: 475.03691; LC-MS: ret. time 6.015.

### Example 47

Methyl 3-(9-bromo-5,5-dioxido-6H-dibenzo[c,e][1,2]thiazin-6-yl)propanoate: To a solution the appropriate compound of formula 5a (0.300 g, 0.92 mmol) in dry THE (12 mL), commercial methyl 3-hydroxypropanoate (0.12 mL, 1.3 mmol) and PPh₃ (0.33 g, 1.3 mmol), were added and the solution was sonicated at 25° C. for 7 min. DEAD (0.20 mL, 1.3 mmol) was then added drop-wise and the solution was sonicated for 18 h at 25° C. (approximately 70% of conversion followed by TLC). The mixture was concentrated under reduced pressure, poured into ice-water, basified with aqueous 10% NaOH to pH 10 in order to remove the residual starting material, and extracted with EtOAc (3×20 mL). The combined organic layers were washed brine, dried, and evaporated to dryness. The obtained brown oil was purified by column chromatography (petroleum ether/EtOAc 7:3) followed by trituration with Et₂O to give the desired title compound of formula 6a (0.100 g, 30%) as a white solid: mp 101-103° C. ¹H NMR (200 MHz, CDCl₃): δ 8.10 (d, J=2.3 Hz, 1H, Ar—H), 7.94-7.83 (m, 2H, Ar—H), 7.70 (dt, J=1.5 and 7.4 Hz, 2H, Ar—H), 7.60-7.50 (m, 2H, Ar—H), 7.28 (d, J=8.8 Hz, 1H, Ar—H), 4.10 (t, J=7.4 Hz, 2H, NCH₂), 3.50 (s, 3H, CH₃), 2.50 (t, J=7.4 Hz, 2H, CH₂).

General procedure of direct amidation of compounds of formula 6a with cyclohexylamine to obtain target compounds of formula 8a (Scheme 1): Using the microwave oven a tube containing a mixture of appropriate dibenzothiazine ethyl acetate or the intermediate in example 44 of general formula 6a (1 equiv.) and cyclohexylamine (4 equiv.) was irradiated at 120° C. for 4 h by setting the following experimental parameters: pressure 5 bar, cooling off, FHT on, solvent absorption normal. The residue was poured into ice-water and acidified with 2 N HCl to pH 3. The obtained precipitate was filtered off and crystallized by EtOH to give the target compound of formula 8a.

### Example 48

2-(9-Bromo-5,5-dioxido-6H-dibenzo[c,e][1,2]thiazin-6-yl)-N-cyclohexylacetamide (SM3): following the general procedure above described the title compound was obtained as white solid in 62% yield: mp 211-213° C. ¹H NMR (400 MHz, CDCl₃): δ 8.20 (brs, 1H, Ar—H), 8.00 (d, J=7.8 Hz, 1H, Ar—H), 7.87 (d, J=7.8 Hz, 1H, Ar—H), 7.75 (t, J=7.5 Hz, 1H, Ar—H), 7.73 (t, J=7.4 Hz, 1H, Ar—H), 7.57 (d, J=7.4 Hz, 1H, Ar—H), 7.12 (d, J=8.7 Hz, 1H, H-7), 6.54 (d, J=7.0 Hz, 1H, NH), 4.42 (s, 2H, NCH₂), 3.90-3.75 (m, 1H, Cy-CH), 1.95-1.75 (m, 2H, Cy-CH), 1.65-1.50 (m, 3H, Cy-CH), 1.45-1.25 (m, 2H, Cy-CH), 1.25-1.05 (m, 3H, Cy-CH). ¹³C NMR (100 MHz, DMSO-d₆): δ 166.29, 137.11, 134.09, 133.49, 133.12, 131.05, 129.18, 128.57, 125.91, 125.59, 122.56, 121.09, 118.62, 51.64, 48.45, 32.45, 25.35, 24.35. HRMS (ESI) m/z [M+H]⁺ calcd. for C₂₀H₂₁BrN₂O₃S: 448.0535, found: 448.0456; LC-MS: ret. time 5.754.

### Example 49

2-(9-Bromo-5,5-dioxido-6H-dibenzo[c,e][1,2]thiazin-6-yl)-N-cyclopentylacetamide (SM4): following the general procedure above described the title compound was obtained, after crystallization by EtOH, as white solid in 43% yield: mp 192-194° C. ¹H NMR (400 MHz, CDCl₃): δ 8.19 (d, J=2.1 Hz, 1H, Ar—H), 8.04 (d, J=7.9 Hz, 1H, Ar—H), 7.99 (d, J=7.9 Hz, 1H, Ar—H), 7.80 (dt, J=1.2 and 7.6 Hz, 1H, Ar—H), 7.69-7.61 (m, 2H, Ar—H), 7.15 (d, J=8.7 Hz, 1H, Ar—H), 6.62 (d, J=7.5 Hz, 1H, NH), 4.46 (s, 2H, NCH₂), 4.31-4.24 (m, 1H, cyclopentyl-CH), 2.00-1.92 (m, 2H, cyclopentyl-CH₂), 1.70-1.55 (m, 4H, cyclopentyl-CH₂), 1.45-1.30 (m, 2H, cyclopentyl-CH₂). ¹³C NMR (100 MHz, CDCl₃): δ166.76, 137.03, 134.04, 133.49, 133.14, 131.00, 129.20, 128.57, 125.90, 125.53, 122.53, 121.04, 118.61, 51.60, 51.49, 32.80, 23.45. HRMS (ESI) m/z [M+H]⁺ calcd. for C₁₉H₁₉BrN₂O₃S: 435.0379, found: 435.03733; LC-MS: ret. time 5.434.

### Example 50

2-(9-Bromo-5,5-dioxido-6H-dibenzo[c,e][1,2]thiazin-6-yl)-N-cycloheptylacetamide (SM5): following the general procedure above described the title compound was obtained, after crystallization by EtOH, as white solid in 51% yield: mp 208-210° C. ¹H NMR (400 MHz, CDCl₃): δ 8.18 (d, J=1.8 Hz, 1H, Ar—H), 8.03 (d, J=7.4 Hz, 1H, Ar—H), 7.98 (d, J=7.9 Hz, 1H, Ar—H), 7.80 (t, J=7.6 Hz, 1H, H-2), 7.70-7.60 (m, 2H, Ar—H), 7.15 (d, J=8.7 Hz, 1H, Ar—H), 6.60 (d, J=7.5 Hz, 1H, NH), 4.45 (s, 2H, NCH₂), 4.20-3.95 (m, 1H, cycloheptyl-CH), 1.90-177 (m, 2H, cycloheptyl-CH₂), 1.70-1.30 (m, 10H, cycloheptyl-CH₂). ¹³C NMR (100 MHz, CDCl₃): δ 166.01, 137.08, 134.08, 133.47, 133.13, 131.04, 129.18, 128.57, 125.92, 125.55, 122.55, 121.04, 118.60, 51.60, 50.63, 34.59, 27.84, 23.72. HRMS (ESI) m/z [M+H]⁺ calcd. for C₂₁H₂₃BrN₂O₃S: 463.0689, found: 463.0688; LC-MS: ret. time 6.018.

### Example 51

3-(9-Bromo-5,5-dioxido-6H-dibenzo[c,e][1,2]thiazin-6-yl)-N-cyclohexylpropanamide (SM10). following the general procedure above described the title compound was obtained, after purification by flash column chromatography (cyclohexane/EtOAc 7:3), as white solid in 34% yield: mp 114-116° C. ¹H NMR (400 MHz, CDCl₃): δ 8.13 (brs, 1H, Ar—H), 8.00 (d, J=7.6 Hz, 1H, Ar—H), 7.90 (d, J=7.7 Hz, 1H, Ar—H), 7.75 (t, J=7.7 Hz, 1H, Ar—H), 7.65-7.60 (m, 2H, Ar—H), 7.42 (d, J=8.7 Hz, 1H, Ar—H), 5.55 (d, J=6.4 Hz, 1H, NH), 4.15 (t, J=6.6 Hz, 2H, NCH₂), 3.75-3.60 (m, 1H, Cy-CH), 2.60 (t, J=6.6 Hz, 2H, CH₂), 1.85-1.53 (m, 5H, Cy-CH₂), 1.50-1.10 (m, 5H, Cy-CH₂). ¹³C NMR (100 MHz, CDCl₃): δ 168.33, 137.37, 134.19, 132.91, 132.21, 130.81, 128.46, 127.79, 126.07, 125.26, 123.13, 122.13, 118.23, 47.96, 45.98, 36.49, 32.36, 24.90, 24.18. HRMS (ESI) calcd for C₂₁H₂₃BrN₂O₃S [M⁺+H]⁺: 463.0689, found: 463.0693; LC-MS: ret. time 4.772

### Example 52

2-(9-chloro-5,5-dioxido-6H-dibenzo[c,e][1,2]thiazin-6-yl)-N-cyclohexylacetamide (SM254): following the general procedure above described the title compound was obtained, after crystallization by EtOH, as white solid in 60% yield: mp 218-220° C. ¹H NMR (400 MHz, CDCl₃): δ 8.02-7.95 (m, 2H, Ar—H), 7.92 (d, J=8.1 Hz, 1H, Ar—H), 7.75 (dt, J=1.2 and 7.7 Hz, 1H, Ar—H), 7.63 (dt, J=0.8 and 8.0 Hz, 1H, Ar—H), 7.43 (dd, J=2.2 and 8.7 Hz, 1H, Ar—H), 7.18 (d, J=8.7 Hz, 1H, H-7), 6.53 (d, J=7.0 Hz, 1H, NH), 4.42 (s, 2H, NCH₂), 3.90-3.75 (m, 1H, Cy-CH), 1.90-1.75 (m, 2H, Cy-CH), 1.65-1.50 (m, 4H, Cy-CH), 1.40-1.25 (m, 2H, Cy-CH), 1.25-1.05 (m, 2H, Cy-CH). ¹³C NMR (100 MHz, CDCl₃): δ166.29, 136.69, 134.16, 133.08, 131.17, 131.11, 130.58, 129.16, 125.88, 125.61, 125.32, 122.58, 120.97, 51.75, 48.45, 32.45, 25.34, 24.32; HRMS (ESI) m/z [M+H]⁺ calcd. for C₂₀H₂₁ClN₂O₃S: 405.1039, found: 404.1032. LC-MS: ret. time 6.492 min.

### Example 53

N-cyclohexyl-2-[5,5-dioxido-9-(trifluoromethyl)-6H-dibenzo[c,e][1,2]thiazin-6-yl]acetamide (SM231): following the general procedure above described the title compound was obtained, after purification by flash column chromatography (cyclohexane/EtOAc 7:3), as white solid in 70% yield: mp 225-226° C. ¹H-NMR (200 MHz, CDCl₃): δ 8.29 (brs, 1H, Ar—H), 8.03 (d, J=7.5 Hz, 1H, Ar—H), 7.81 (d, J=7.8 Hz, 1H, Ar—H), 7.75-7.65 (m, 2H, Ar—H), 7.35 (d, J=8.4 Hz, 1H, Ar—H), 6.57 (brs, 1H, NH), 4.52 (s, 2H, NCH₂), 3.85-3.75 (m, 1H, Cy-CH), 1.85-1.75 (m, 2H, Cy-CH), 1.65-1.48 (m, 3H, Cy-CH), 1.40-1.25 (m, 2H, Cy-CH), 1.20-1.10 (m, 3H, Cy-CH). ¹³C NMR (100 MHz, CDCl₃): δ 166.04, 140.60, 134.01, 133.29, 131.16, 129.36, 127.33 (q, J_{C-F}=33.1 Hz, C-9), 127.32 (d, J_{C-F}=3.5 Hz, C-10), 126.02, 123.86, 123.63 (q, J_{C-F}=270.7 Hz, CF₃), 122.96 (d, J_{C-F}=3.8 Hz, C-8), 119.49, 51.25, 48.55, 32.43, 25.32, 24.33. HRMS (ESI) m/z [M+Na]⁺ calcd. for C₂₁H₂₁F₃N₂O₃S: 461.1118, found: 461.1124. LC-MS: ret. time 4.688 min.

### Example 54

N-cyclohexyl-2-[9-(methylthio)-5,5-dioxido-6H-dibenzo[c,e][1,2]thiazin-6-yl]acetamide (SM340): following the general procedure above described the title compound was obtained, after crystallization by EtOH, as white solid in 63% yield: mp 178-180° C. ¹H-NMR (400 MHz, CDCl₃): δ 8.03-7.98 (m, 2H, Ar—H), 7.92 (d, J=1.6 Hz, 1H, Ar—H), 7.78 (t, J=7.6 Hz, 1H, Ar—H), 7.64 (t, J=7.4 Hz, 1H, Ar—H), 7.39 (dd, J=1.9 and 8.6 Hz, 1H, Ar—H), 7.20 (d, J=8.6 Hz, 1H, Ar—H), 6.60 (d, J=7.5 Hz, 1H, NH), 4.44 (s, 2H, NCH₂), 3.90-3.75 (m, 1H, Cy-CH), 2.58 (s, 3H, SCH₃), 1.90-1.80 (m, 2H, Cy-CH), 1.75-1.50 (m, 3H, Cy-CH), 1.40-1.25 (m, 2H, Cy-CH), 1.20-1.10 (m, 3H, Cy-CH). ¹³C-NMR (100 MHz, CDCl₃): δ166.60, 135.92, 135.61, 134.20, 132.99, 131.79, 129.14, 128.79, 125.81, 124.44, 124.07, 122.59, 120.25, 51.84, 48.39, 32.46, 25.35, 24.35, 16.44. HRMS (ESI) m/z [M+H]⁺ calcd for C₂₁H₂₄N₂O₃S₂: 417.1309, found: 417.1305; LC-MS: ret. time 5.403 min.

### Example 55

N-Cyclohexyl-2-(9-methoxy-5,5-dioxido-6H-dibenzo[c,e][1,2]thiazin-6-yl)acetamide (SM225): following the general procedure above described the title compound was obtained, after purification by flash column chromatography (cyclohexane/EtOAc 7:3), as white solid in 45% yield: mp 216-217° C. ¹H-NMR (400 MHz, CDCl₃): δ 7.99-7.93 (m, 2H, Ar—H), 7.74 (t, J=7.6 Hz, 1H, Ar—H), 7.59 (t, J=7.6 Hz, 1H, Ar—H), 7.49 (d, J=2.6 Hz, 1H, Ar—H), 7.20 (d, J=8.9 Hz, 1H, Ar—H), 7.02 (dd, J=2.7 and 8.9 Hz, 1H, Ar—H), 6.59 (d, J=7.8 Hz, 1H, NH), 4.32 (s, 2H, NCH₂), 3.88-3.75 (m, 4H, OCH₃, and Cy-CH), 1.85-1.75 (m, 2H, Cy-CH), 1.65-1.50 (m, 3H, Cy-CH), 1.35-1.25 (m, 2H, Cy-CH), 1.20-1.10 (m, 3H, Cy-CH). ¹³C-NMR (100 MHz, CDCl₃): δ166.82, 157.36, 134.29, 132.96, 132.22, 131.86, 128.76, 125.87, 125.50, 122.84, 121.68, 116.50, 110.77, 55.78, 52.75, 48.27, 32.52, 25.39, 24.43. HRMS (ESI) m/z [M+H]⁺ calcd for C₂₁H₂₄N₂O₄S: 401.1539, found: 401.1533; LC-MS: ret. time 5.544 min.

### Example 56

2-(7-Bromo-5,5-dioxido-6H-dibenzo[c,e][1,2]thiazin-6-yl)-N-cyclohexylacetamide (SM227): following the general procedure above described the title compound was obtained, after purification by flash column chromatography (cyclohexane/EtOAc 6:4), as white solid in 40% yield:mp 159-160° C. ¹H-NMR (200 MHz, DMSO-d₆) δ 8.15 (d, 2H, H-4 and H-8), 7.70-7.85 (m, 4H, H-1, H-9, H-10 and NH), 7.55-7.70 (t, J=7.4 Hz, 1H, H-2), 7.40 (t, J=7.9 Hz, 1H, H-3), 4.40 (brs, 2H, NCH₂), 3.00-3.10 (m, 1H, cyclohexyl CH), 0.60-1.60 (m, 10H, cyclohexyl CH₂). ¹³C-NMR (100 MHz, CDCl₃): δ 165.51, 139.33, 134.86, 133.55, 133.17, 132.58, 130.40, 129.51, 129.50, 126.35, 125.40, 125.09, 121.45, 54.69, 48.36, 32.70, 25.52, 25.40. HRMS (ESI) m/z [M+H]⁺ calcd for C₂₀H₂₁BrN₂O₃S: 448.0539, found: 448.0267; LC-MS: ret. time 4.10 min.

### Example 57

2-(8-Bromo-5,5-dioxido-6H-dibenzo[c,e][1,2]thiazin-6-yl)-N-cyclohexylacetamide (SM228) and 2-(10-bromo-5,5-dioxido-6H-dibenzo[c,e][1,2]thiazin-6-yl)-N-cyclohexylacetamide (SM229): following the general procedure above described a mixture of the two regioisomers of formula 6a was reacted with cyclohexylamine obtaining the two regioisomers of formula 8a that were separated by flash column chromatography (CH₂Cl₂/acetone 98:2) and each compound was further purified by crystallization with EtOH to afford target compounds SM228 (R_{f}>by TLC) and SM229 (R_{f}<by TLC).

SM228: 8% yield: mp 184-185° C. ¹H-NMR (400 MHz, CDCl₃): δ 8.10-7.80 (m, 3H, Ar—H), 7.82 (t, J=7.4 Hz, 1H, Ar—H), 7.65 (t, J=7.6 Hz, 1H, Ar—H), 7.52 (dd, J=1.5 and 8.5 1H, Ar—H), 7.40 (brs, 1H, Ar—H), 6.55 (d, J=8.0 Hz, 1H, NH), 4.50 (s, 2H, NCH₂), 4.00-3.75 (m, 1H, Cy-CH), 2.00-1.80 (m, 2H, Cy-CH₂), 1.75-1.50 (m, 2H, Cy-CH₂), 1.45-1.00 (m, 6H, Cy-CH₂). ¹³C NMR (100 MHz, DMSO-d₆): δ 165.60, 140.02, 134.99, 133.22, 131.50, 129.45, 128.16, 127.94, 126.66, 123.86, 123.82, 123.30, 121.76, 50.06, 48.12, 32.67, 25.53, 24.76. HRMS (ESI) m/z [M+Na]⁺ calcd for C₂₀H₂₁BrN₂O₃S: 471.0354, found: 471.041; LC-MS: ret. time 12.592 min.

SM229: 21% yield: mp 211-212° C. ¹H-NMR (400 MHz, CDCl₃): δ 8.62 (d, J=8.3 Hz, 1H, Ar—H), 8.00 (dd, J=1.4 and 7.8 Hz, 1H, Ar—H), 7.75-7.65 (m, 4H, Ar—H), 7.28-7.24 (m, 2H, Ar—H), 6.50 (d, J=8.1 Hz, 1H, NH), 4.40 (s, 2H, NCH₂), 3.80-3.70 (m, 1H, Cy-CH), 1.90-1.80 (m, 2H, Cy-CH₂), 1.75-1.50 (m, 2H, Cy-CH₂), 1.45-1.00 (m, 6H, Cy-CH₂). NMR COSY spectrum showed two relevant NOE cross-peaks: H-9 (δ 7.70, dd)→H-8 (δ 7.32, t), H-9→H-7 (δ 7.28, dd). NMR NOESY spectrum showed one relevant NOE cross-peak: H-8→NCH₂. ¹³C NMR (100 MHz, DMSO-d₆): δ 165.49, 140.92, 134.85, 131.81, 131.48, 131.19, 131.13, 130.37, 129.41, 125.44, 121.64, 121.38, 120.59, 50.77, 48.11, 32.66, 25.52, 24.76. HRMS (ESI) m/z [M+Na]⁺ calcd for C₂₀H₂₁BrN₂O₃S: 471.0354, found: 471.0407; LC-MS: ret. time 12.893 min.

### Example 58

2-(8-chloro-5,5-dioxido-6H-dibenzo[c,e][1,2]thiazin-6-yl)-N-cyclohexylacetamide (SM586) and 2-(10-chloro-5,5-dioxido-6H-dibenzo[c,e][1,2]thiazin-6-yl)-N-cyclohexylacetamide (SM585): following the general procedure above described a mixture of the two appropriate regioisomers of formula 6a was reacted with cyclohexylamine obtaining the two regioisomers of formula 8a that were separated by flash column chromatography (CH₂Cl₂/acetone 98:2) and each compound was further purified by crystallization with EtOH to afford target compounds SM586 (R_{f}>by TLC) and SM585 (R_{f}<by TLC).

SM586: 18% yield: mp 193-195° C. ¹H-NMR (400 MHz, CDCl₃): δ8.02-7.90 (m, 3H, Ar—H), 7.74 (dt, J=1.2 and 7.7 Hz, 1H, Ar—H), 7.60 (dt, J=0.7 and 8.0 Hz, 1H, Ar—H), 7.33 (dd, J=2.0 and 8.2 Hz, 1H, Ar—H), 7.28 (d, J=2.0 Hz, 1H, Ar—H), 6.50 (d, J=7.5 Hz, 1H, NH), 4.49 (s, 2H, NCH₂), 3.90-3.75 (m, 1H, Cy-CH), 1.90-1.80 (m, 2H, Cy-CH₂), 1.75-1.50 (m, 4H, Cy-CH₂), 1.45-1.35 (m, 2H, Cy-CH₂), 1.20-1.05 (m, 2H, Cy-CH₂); ¹³C-NMR (100 MHz, CDCl₃): δ 166.05, 139.07, 136.51, 133.87, 133.07, 131.56, 128.74, 126.80, 125.71, 125.68, 122.52, 122.41, 119.71, 51.57, 48.41, 32.41, 25.36, 24.30; HRMS (ESI) m/z [M+H]⁺ calcd. for C₂₀H₂1ClN₂O₃S: 405.1039, found:405.1037; LC-MS: ret. time 5.628 min.

SM585: 15% yield: mp 200-202° C. ¹H-NMR (400 MHz, CDCl₃): δ 8.60 (d, J=8.0 Hz, 1H, Ar—H), 7.95 (dd, J=1.2 and 8.5 Hz, 1H, Ar—H), 7.70 (dt, J=1.3 and 8.5 Hz, 1H, Ar—H), 7.59 (dd, J=1.2 and 8.0 Hz, 1H, Ar—H), 7.43 (dd, J=1.1 and 8.1 Hz, 1H, Ar—H), 7.34 (t, J=8.1 Hz, 1H, Ar—H), 7.20 (dd, J=1.1 and 8.1 Hz, 1H, Ar—H), 6.35 (d, J=7.0 Hz, 1H, NH), 4.48 (s, 2H, NCH₂), 3.90-3.75 (m, 1H, Cy-CH), 1.90-1.80 (m, 2H, Cy-CH₂), 1.75-1.50 (m, 4H, Cy-CH₂), 1.45-1.35 (m, 2H, Cy-CH₂), 1.20-1.05 (m, 2H, Cy-CH₂); ¹³C-NMR (100 MHz, CDCl₃): δ 166.25, 139.99, 135.69, 132.59, 131.57, 130.35, 130.16, 130.02, 128.78, 128.70, 123.48, 122.29, 118.69, 52.15, 48.38, 32.45, 25.34, 24.33; HRMS (ESI) m/z [M+H]⁺ calcd. for C₂₀H₂₁ClN₂O₃S: 405.1039, found: 405.1037; LC-MS: ret. time 5.631 min.

### Example 59

N-cyclohexyl-2-[5,5-dioxido-8-(trifluoromethyl)-6H-dibenzo[c,e][1,2]thiazin-6-yl]acetamide (SM338) and N-cyclohexyl-2-[5,5-dioxido-10-(trifluoromethyl)-6H-dibenzo[c,e][1,2]thiazin-6-yl]acetamide (SM339): following the general procedure above described a mixture of the two appropriate regioisomers of formula 6a was reacted with cyclohexylamine obtaining the two regioisomers of formula 8a that were separated by flash column chromatography(CH₂Cl₂/acetone 99:1) and each compound was further purified by crystallization with EtOH to afford target compounds SM338 (R_{f}>by TLC) and SM339 (R_{f}<by TLC).

SM338: 40% yield: mp 230-232° C. ¹H-NMR (400 MHz, CDCl₃): δ 8.20-8.10 (d, J=8.2 Hz, 1H, Ar—H), 8.05-7.98 (m, 2H, Ar—H), 7.78 (dt, J=1.5 and 7.5 Hz, 1H, Ar—H), 7.78 (dt, J=1.5 and 7.5 Hz, 1H, Ar—H), 7.69 (dt, J=1.3 and 7.7 Hz, 1H, Ar—H), 7.65 (d, J=8.0 Hz, 1H, Ar—H), 7.50 (brs, 1H, Ar—H), 6.50 (d, J=6.9 Hz, 1H, NH), 4.48 (s, 2H, NCH₂), 3.90-3.75 (m, 1H, Cy-CH), 1.90-1.80 (m, 2H, Cy-CH₂), 1.75-1.50 (m, 4H, Cy-CH₂), 1.45-1.35 (m, 2H, Cy-CH₂), 1.20-1.05 (m, 2H, Cy-CH₂); ¹³C-NMR (100 MHz, CDCl₃): δ 165.86, 138.57, 134.66, 133.16, 132.50 (q, J_{C-F}=33.1 Hz, C-8), 131.11, 129.58, 127.08, 126.42, 126.27, 123.79 (q, J_{C-F}=271.0 Hz, CF₃), 122.59, 121.89 (q, J_{C-F}=5 Hz, C-9), 116.83 (q, J_{C-F}=6 Hz, C-7), 51.71, 48.44, 32.40, 25.33, 24.33; HRMS (ESI) m/z [M+H]⁺ calcd for C₂₁H₂₁F₃N₂O₃S: 439.1303, found: 439.1296; LC-MS: ret. time 6.892 min.

SM339: 26% yield: mp 211-212° C.; ¹H-NMR (400 MHz, CDCl₃): δ 8.03-7.95 (m, 2H, Ar—H), 7.82-7.58 (m, 4H, Ar—H), 7.51 (d, J=8.3 Hz, 1H, Ar—H), 6.35 (d, J=8.0 Hz, 1H, NH), 4.40 (s, 2H, NCH₂), 4.00-3.75 (m, 1H, Cy-CH), 1.90-1.80 (m, 2H, Cy-CH₂), 1.75-1.50 (m, 4H, Cy-CH₂), 1.45-1.05 (m, 4H, Cy-CH₂); ¹³C-NMR (100 MHz, CDCl₃): δ 168.08, 140.01 (brs, C-6a), 139.80, 134.90, 134.32, 132.30, 130.56, 127.7 (d, J_{C-F}=2.0 Hz, C-8), 124.57, 122.30 (q, J_{C-F}=29.0 Hz, C-10), 119.60 (q, J_{C-F}=270.1 Hz, CF₃), 117.80 (q, J_{C-F}=3.1 Hz, C-9), 115.85 (q, J_{C-F} 2.0 Hz, C-10a), 115.09, 52.02, 49.50, 32.40, 26.28, 24.12; HRMS (ESI) m/z [M+H]⁺ calcd for C₂₁H₂₁F₃N₂O₃S: 439.1303, found: 439.1298; LC-MS: ret. time 6.598 min.

### Example 60

2-(8-chloro-9-fluoro-5,5-dioxido-6H-dibenzo[c,e][1,2]thiazin-6-yl)-N-cyclohexylacetamide (SM336) and 2-(10-chloro-9-fluoro-5,5-dioxido-6H-dibenzo[c,e][1,2]thiazin-6-yl)-N-cyclohexylacetamide (SM337): following the general procedure above described a mixture of the two appropriate regioisomers of formula 6a was reacted with cyclohexylamine obtaining the two regioisomers of formula 8a that were separated by flash column chromatography (cyclohexane/EtOAc 7:3) followed by crystallization with EtOH to afford target compounds SM336 (R_{f}>by TLC) and SM337 (R_{f}<by TLC).

SM336: 21% yield: mp 211-213° C. ¹H-NMR (400 MHz, DMSO-d₆): δ 7.96 (dd, J=1.5 and 7.0 Hz, 1H, Ar—H), 7.8 (d, J=8.2 Hz, 1H, Ar—H), 7.78-7.70 (m, 2H, Ar—H), 7.61 (dt, J=1.4 and 7.7 Hz, 1H, Ar—H), 7.33 (d, J=6.4 Hz, 1H, Ar—H), 6.4 (d, J=9.0 Hz, 1H, Ar—H), 4.35 (s, 2H, NCH₂), 3.80-3.70 (m, 1H, Cy-CH), 1.85-1.75 (m, 2H, Cy-CH₂), 1.75-1.45 (m, 2H, Cy-CH₂), 1.45-1.00 (m, 6H, Cy-CH₂). ¹³C NMR (100 MHz, DMSO-d₆): δ 165.59, 154.86 (d, J_{C-F}=243.0 Hz, C-9), 136.05 (d, J_{C-F}=2.6 Hz, C-6a), 135.17, 133.19, 130.80, 130.05, 127.16, 125.69 (d, J_{C-F}=8.0 Hz, C-10a), 123.97, 121.84, 121.29 (d, J_{C-F}=20.0 Hz, C-8), 113.96 (d, J_{C-F}=24.0 Hz, C-10), 50.97, 48.12, 32.62, 25.51, 24.73. HRMS (ESI) m/z [M+H]⁺ calcd. for C₂₀H₂OClFN₂O₃S: 423.0946, found: 423.0938; LC-MS: ret. time 6.560 min.

SM337: 53% yield: mp 216-217° C. ¹H-NMR (400 MHz, CDCl₃): δ 8.51 (d, J=8.2 Hz, 1H, Ar—H), 8.00 (d, J=7.7 Hz, 1H, Ar—H), 7.73 (dt, J=1.2 and 7.5 Hz, 1H, Ar—H), 7.64 (t, J=7.5 Hz, 1H, Ar—H), 7.35-7.20 (m, 2H, Ar—H), 6.25 (d, J=7.1 Hz, 1H, NH), 4.25 (s, 2H, NCH₂), 3.80-3.70 (m, 1H, Cy-CH), 1.90-1.80 (m, 2H, Cy-CH₂), 1.75-1.50 (m, 4H, Cy-CH₂), 1.40-1.00 (m, 4H, Cy-CH₂). ¹³C NMR (100 MHz, CDCl₃): δ 166.08, 156.77 (d, J_{C-F}=246.1 Hz, C-9), 135.85 (brs, C-6a), 135.81, 131.72, 129.99, 129.81 (d, J_{C-F}=2.8 Hz, C-10a), 129.39, 125.44, 122.69, 120.41 (J_{C-F}=8.1 Hz, C-7), 119.5 (J_{C-F}=20.1 Hz, C-10), 117.43 (J_{C-F}=24.0 Hz, C-8), 52.73, 48.45, 32.53, 25.33, 24.39; HRMS (ESI) m/z [M+H]⁺ calcd. for C₂₀H₂OClFN₂O₃S: 423.0946, found: 423.0938; LC-MS: ret. time 6.520 min.

### Example 61

N-cyclohexyl-2-(8,10-dichloro-5,5-dioxido-6H-dibenzo[c,e][1,2]thiazin-6-yl)acetamide (SM587): following the general procedure above described the title compound was obtained, after crystallization by EtOH, as white solid in 50% yield: mp 212.0-213.0° C.; H-NMR (200 MHz, DMSO-d₆) δ 8.50 (d, J=8.0 Hz, 1H, Ar—H), 7.97 (dd, J=1.3 and 7.8, Hz, 1H, Ar—H), 7.71 (dt, J=1.4 and 8.0 Hz, 1H, Ar—H), 7.61 (dt, J=0.9 and 7.6, Hz, 1H, Ar—H), 7.45 (d, J=2.0 Hz, 1H, Ar—H), 7.22 (d, J=2.0 Hz, 1H, Ar—H), 6.28 (d, J=7.6 Hz, 1H, NH), 4.31 (s, 2H, NCH₂), 3.80-3.70 (m, 1H, Cy-CH), 1.90-1.80 (m, 2H, Cy-CH₂), 1.75-1.50 (m, 4H, Cy-CH₂), 1.40-1.00 (m, 4H, Cy-CH₂); ¹³C NMR (100 MHz, CDCl₃): δ 165.74, 140.50, 135.53, 135.44, 133.58, 131.78, 129.81, 129.75, 129.03, 128.46, 122.36, 122.06, 119.02, 52.00, 48.47, 32.46, 25.32, 24.37; HRMS (ESI) m/z [M+H]⁺ calcd. for C₂₀H₂₀Cl₂N₂O₃S: 439.0649, found: 439.0646; LC-MS: ret. time 1.920 min.

### Example 62

2-(5,5-dioxido-6H-dibenzo[c,e][1,2]thiazin-6-yl)-N-cyclohexylacetamide (SM7): to a suspension of LiAlH₄ (0.021 g, 0.55 mmol) in dry THE (1 mL) cooled to 0° C., a solution of SM3 (0.100 g, 0.22 mmol) in dry THE (4 mL) was added drop-wise under N₂ and then the mixture was stirred at 50° C. for 2 h. After cooling and quenching with EtOAc followed MeOH, the mixture was then poured into ice-water and extracted with EtOAc (3×20 mL). The combined organic layers were washed with brine, dried, and evaporated to dryness. The crude colorless oil obtained was purified by flash column chromatography, eluting with cyclohexane/EtOAc (7:3), to give SM7 (0.040 g, 49%) as a white solid: mp 176-178° C. ¹H NMR (400 MHz, CDCl₃): δ 8.25-8.00 (m, 3H, Ar—H), 7.77 (dt, J=1.2 and 8.4 Hz, 1H, Ar—H), 7.62 (t, J=7.7 Hz, 1H, Ar—H), 7.51 (dt, J=1.3 and 8.6 Hz, 1H, Ar—H), 7.39 (dt, J=1.0 and 8.4 Hz, 1H, Ar—H), 7.25 (dd, J=1.8 and 7.2 Hz, 1H, Ar—H), 6.60 (brs, 1H, NH), 4.48 (s, 2H, N—CH₂), 3.91-3.83 (m, 1H, Cy-CH), 1.90-1.80 (m, 2H, Cy-CH₂), 1.60-1.50 (m, 3H, Cy-CH), 1.40-1.25 (m, 2H, Cy-CH₂), 1.20-1.00 (m, 3H, Cy-CH). ¹³C NMR (100 MHz, CDCl₃): δ 166.18, 137.66, 133.57, 132.43, 131.81, 130.31, 127.98, 125.31, 125.21, 124.88, 123.36, 121.96, 118.99, 51.19, 47.85, 31.92, 24.87, 23.81. HRMS (ESI) calcd for C₂₀H₂₂N₂O₃S [M⁺+H]⁺: 371.1429, found: 371.1397. LC-MS: ret. time 5.212.

### Example 63

N-Cyclohexyl-2-(9-hydroxy-5,5-dioxido-6H-dibenzo[c,e][1,2]thiazin-6yl)acetamide (SM226): to a solution of target compound SM225 (0.22 g. 0.55 mmol) in dry CH₂Cl₂ (12 mL) and under N₂ flux, 1M BBr₃ in CH₂Cl₂ (2.75 g, 2.75 mmol) was added dropwise at −60° C. and then the solution was stirred at −30° C. for 12 h. After quenching of the excess of BBr₃ with MeOH, H₂O, and saturated solution of NaHCO₃, the mixture was acidified with 2N HCl to pH and extracted with CH₂Cl₂ (3×30 mL). The combined organic layers were washed with brine, dried, and evaporated to dryness and the residue which was purified by flash column chromatography (CHCl₃/MeOH 95:5), to give compound SM226, as white solid in 88% yield: mp 216-217° C. ¹H-NMR (400 MHz, DMSO-d₆): δ 9.77 (s, 1H, OH), 8.15 (d, J=8.9 Hz, 1H, Ar—H), 7.90-7.74 (m, 3H, Ar—H and NH), 7.62 (t, J=7.6 Hz, 1H, Ar—H), 7.45 (d, J=2.5 Hz, 1H, Ar—H), 7.25 (d, J=8.8 Hz, 1H, Ar—H), 6.85 (dd, J=2.6 and 8.7 Hz, 1H, Ar—H), 4.30 (s, 2H, NCH₂), 3.40-3.30 (m, 1H, Cy-CH), 1.75-1.40 (m, 5H, Cy-CH₂), 1.30-0.90 (m, 5H, Cy-CH₂). ¹³C-NMR (100 MHz, DMSO-d₆): δ 166.19, 154.95, 143.08, 135.01, 133.28, 132.00, 129.29, 126.41, 126.31, 123.94, 121.99, 118.10, 111.45, 51.74, 48.13, 32.38, 25.31, 24.59. HRMS (ESI) m/z [M+H]⁺ calcd for C₂₀H₂₂N₂O₄S: 387.1380, found: 387.1372; LC-MS: ret. time 4.691 min.

### Example 64

N-Cyclohexyl-2-[9-[2-(dimethylamino)ethoxy]-5,5-dioxido-6H-dibenzo[c,e][1,2]thiazin-6-yl]acetamide (SM230): to a solution of target compound SM226 (0.18 g. 0.47 mmol) in dry DMF (7 mL), Cs₂CO₃ (0.23 g. 0.70 mmol) and commercial 1-chloro-N,N-dimethylethanamine hydrochloride (0.07 g. 0.47 mmol) were added and the mixture was maintained under magnetic stirring at 85° C. for 2 h. The mixture was poured into ice-water, extracted with EtOAc (3×20 mL) and the combined organic layers were washed with brine, dried and evaporated to dryness to give an oil which was purified by flash column chromatography (CHCl₃/MeOH 9:1), to afford SM230 as low melting solid in 57% yield: mp 66-67° C. ¹H-NMR (200 MHz, DMSO-d₆): δ 8.23 (d, J=8.1 Hz, 1H, Ar—H), 7.90-7.65 (m, 3H, Ar—H and NH), 7.60-7.55 (m, 2H, Ar—H), 7.28 (d, J=8.9 Hz, 1H, Ar—H), 7.10 (dd, J=2.5 and 9.0 Hz, 1H, Ar—H), 4.40 (s, 2H, SO₂N—CH₂), 4.20 (t, J=5.3 Hz, 2H, OCH₂), 3.90-3.80 (m, 1H, Cy-CH), 2.80 (t, J=5.3 Hz, 2H, NCH₂), 2.40 (s, 6H, NCH₃), 1.75-1.40 (m, 5H, Cy-CH₂), 1.30-0.90 (m, 5H, Cy-CH₂). ¹³C-NMR (100 MHz, DMSO-d₆): δ 165.84, 156.34, 135.42, 132.86, 132.29, 132.26, 129.15, 126.89, 126.26, 123.65, 121.87, 117.63, 110.75, 66.68, 51.40, 48.04, 45.99, 32.66, 25.53, 24.77. HRMS (ESI) m/z [M+H]⁺ calcd for C₂₄H₃₁N₃O₄S: 458.2200, found: 458.2200; LC-MS: ret. time 6.360 min.

Experimental procedure for making compounds of formula 7a in Scheme 1 are described below.

### Example 65

2-(9-Bromo-5,5-dioxido-6H-dibenzo[c,e][1,2]thiazin-6-yl)acetic acid of formula 7a: A stirred mixture of ethyl 2-(9-bromo-5,5-dioxido-6H-dibenzo[c,e][1,2]thiazin-6-yl)acetate of formula 6a (Example 36; 0.600 g, 1.5 mmol) in aqueous 10% NaOH (7 mL) and EtOH (7 mL) was refluxed for 30 min, then cooled, concentrated under reduced pressure, poured into ice-water and acidified with 2N HCl to pH 2. The formed precipitate was filtered off to give the compound (0.540 g, 96%) as a white solid that was used as is in the next reaction step: mp 207-209° C. ¹H NMR (400 MHz, DMSO-d₆): δ 8.40 (d, J=2.2 Hz, 1H, Ar—H), 8.31 (d, J=8.0 Hz, 1H, Ar—H), 7.91 (dd, J=1.1 and 7.7 Hz, 1H, Ar—H), 7.84 (dt, J=1.3 and 7.7 Hz, 1H, Ar—H), 7.75-7.69 (m, 2H, Ar—H), 7.45 (d, J=8.7 Hz, 1H, H-7), 4.80 (s, 2H, NCH₂).

### Example 66

3-Fluoro-5,5-dioxido-9-(trifluoromethyl)-6H-dibenzo[c,e][1,2]thiazin-6-yl]acetic acid of formula 7a: to a solution of ethyl [3-fluoro-5,5-dioxido-9-(trifluoromethyl)-6H-dibenzo[c,e][1,2]thiazin-6-yl]acetate of formula 6a (Example 43; 1.25 g, 3.09 mmol) in dioxane (25 mL), a solution of 1N LiOH monohydrate (2.47 mL) was added. The reaction mixture was stirred at room temperature for 10 min. and then poured into ice-water and acidified with 2N HCl (pH=2). The precipitate formed was filtered and dried to give the desired compound as white solid (1.16 g, 96%). ¹H NMR (400 MHz, CDCl₃): δ 4.70 (s, 1H, NCH₂), 7.35 (d, J=8.5 Hz, 1H, H-7), 7.40-7.50 (m, 1H, H-2), 7.60-7.65 (m, 1H, H-4), 7.70 (d, J=8.5 Hz, 1H, H-8), 8.00 (dd, J=4.5 and 8.8 Hz, 1H, H-1), 8.20 (s, 1H, H-10).

### Example 67

2-(9-bromo-5,5-dioxo-6H-dibenzo[c,e][1,2]thiazin-6 (5H)-yl)-N-phenylacetamide (SM6). A mixture of 2-(9-Bromo-5,5-dioxido-6H-dibenzo[c,e][1,2]thiazin-6-yl)acetic acid of general formula 7a (Example 65) (0.530 g, 1.44 mmol) and SOCl₂ (2 mL) was refluxed under magnetic stirring for 1 h, then the excess of SOCl₂ was removed by distillation and the residue was washed 3 times with dry toluene. The obtained acyl chloride was solubilized in dry DMF (7 mL) and added drop-wise, under N₂ atmosphere, to a stirred solution of aniline (0.264 mL, 2.88 mmol) and Et₃N (0.401 mL, 2.88 mmol) in dry DMF (3 mL) at room temperature. The mixture was left under magnetic stirring overnight then poured into ice-water and acidified with 2N HCl to pH 3. The precipitate was filtered and purified by flash column chromatography, eluting with CHCl₃, to give target compound SM6 (0.150 g, 25%) as a white solid: mp 128-130° C. ¹H NMR (400 MHz, CDCl₃): (8.37 (bs, 1H, NH), 8.20 (d, J=2.1 Hz, 1H, Ar—H), 8.10 (d, J=7.7 Hz, 1H, Ar—H), 8.00 (d, J=7.7 Hz, 1H, Ar—H), 7.80 (t, J=7.7 Hz, 1H, Ar—H), 7.70 (t, J=7.7 Hz, 1H, Ar—H), 7.60 (dd, J=2.2 and 7.8 Hz, 1H, Ar—H), 7.55-7.48 (m, 2H, Ar—H), 7.35-7.30 (m, 2H, Ar—H), 7.20 (d, J=7.8 Hz, 1H, Ar—H), 7.10 (t, J=7.4 Hz, 1H, Ar—H), 4.52 (s, 2H, CH₂). ¹³C NMR (100 MHz, CDCl₃): δ 165.47, 137.03, 136.86, 133.88, 133.75, 133.33, 131.09, 129.30, 129.06, 128.73, 126.02, 125.66, 125.09, 122.76, 121.21, 120.04, 118.98, 52.02. HRMS (ESI) m/z [M+H]⁺ calcd. for C₂₀H₁₅BrN₂O₃S: 443.0069, found: 443.0057; LC-MS: ret. time 5.571.

### Example 68

2-(9-Bromo-5,5-dioxido-6H-dibenzo[c,e][1,2]thiazin-6-yl)-N-cyclohexyl-N-methylacetamide. The appropriate compound of general formula 7a (2-(9-Bromo-5,5-dioxido-6H-dibenzo[c,e][1,2]thiazin-6-yl)acetic acid; Example 65) (0.59 g, 1.6 mmol) was chlorinated as above reported and the corresponding acyl chloride, solubilized in dry DMF (8 mL), was added drop-wise, under N₂ atmosphere, to a solution of N-methylcyclohexylamine (0.83 mL, 6.4 mmol) in dry DMF (2 mL) at rt. The mixture was heated to 40° C. for 1.5 h, then poured into ice-water, and acidified with 2N HCl to pH 3. The precipitate was filtered and purified by flash column chromatography, eluting with CHCl₃, and subsequent trituration with petroleum ether/Et₂O to give target compound SM8 (0.197 g, 30%) as a white solid: mp 170-172° C. ¹H NMR (400 MHz, DMSO-d₆): (mixture of rotamers) δ 8.42 (d, J=1.7 Hz, 1H, Ar—H), 8.30 (d, J=8.0 Hz, 1H, Ar—H), 7.87 (d, J=7.8 Hz, 1H, Ar—H), 7.82 (t, J=7.6 Hz, 1H, Ar—H), 7.75-7.65 (m, 2H, Ar—H), 7.43 (t, J=8.9 Hz, 1H, H-7), 4.97 (s, 0.88H, NCH₂), 4.90 (s, 1.12H, NCH₂), 4.00-3.90 (m, 0.54H, Cy-CH), 3.60-3.50 (m, 0.46H, Cy-CH), 2.80 (s, 1.68H, NCH₃), 2.60 (s, 1.32H, NCH₃), 1.75-0.95 (m, 10H, Cy-CH₂). ¹³C NMR (100 MHz, DMSO-d₆): (mixture of rotamers) δ 165.91, 165.87, 138.45, 138.32, 135.56, 135.52, 133.15, 133.11, 132.94, 132.91, 131.09, 129.59, 128.36, 126.96, 126.92, 123.76, 123.73, 121.54, 121.48, 117.71, 117.65, 55.11, 52.84, 50.10, 30.59, 29.48, 28.68, 27.38, 25.61, 25.45, 25.30, 25.15. HRMS (ESI) calcd for C₂₁H₂₃BrN₂O₃S [M⁺+H]⁺: 463.0692, found: 463.0678. LC-MS: ret. time 6.034. The two rotamers collapsed to one molecule after recording the NMR spectrum at 50° C.

### Example 69

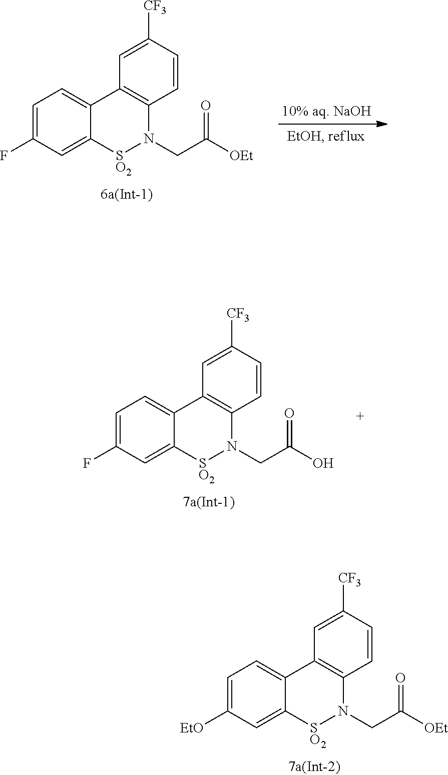 [SMILES: CCOC(=O)CN1c2ccc(C(F)(F)F)cc2-c2ccc(F)cc2S1(=O)=O ; O=C(O)CN1c2ccc(C(F)(F)F)cc2-c2ccc(F)cc2S1(=O)=O ; CCOC(=O)CN1c2ccc(C(F)(F)F)cc2-c2ccc(OCC)cc2S1(=O)=O]

2-(3-fluoro-5,5-dioxido-9-(trifluoromethyl)-6H-dibenzo[c,e][1,2]thiazin-6-yl)acetic acid (7a(Int-1)) and 2-(3-ethoxy-5,5-dioxido-9-(trifluoromethyl)-6H-dibenzo[c,e][1,2]thiazin-6-yl)acetic acid (7a(Int-2)): A stirred mixture of compound of formula 6a(Int-1) (0.40 g, 0.99 mmol) in aqueous 10% NaOH (3 mL) and EtOH (3 mL) was refluxed for 30 min. After cooling, the organic solvent was evaporated under reduced pressure and the residue was poured into ice-water and acidified with 2N HCl (pH=2). The formed precipitate was filtered off to give a mixture of two compounds (7a(Int-1) and 7a(Int-2) in a 1:1 ratio as highlighted by the presence of two spots in TLC (CHCl₃:MeOH 8:2) and also confirmed by ¹H-NMR spectrum. ¹H NMR (400 MHz, CDCl₃): δ 8.20 (bs, 0.5H, H-10), 8.17 (bs, 0.5H, H-10), 7.90 (dd, J=5 and 9 Hz, 0.5H, H-1), 7.85 (d, J=9 Hz, 0.5H, H-1), 7.73-7.60 (m, 1H, H-4 and H-8), 7.60 (d, J=8.5 Hz, 0.5H, H-8), 7.45 (m, 0.5H, H-2), 7.40 (s, 0.5H, H-4), 7.35 (d, J=8 Hz, 0.5H, H-7), 7.20-7.30 (m, 1H, H-2 and H-7), 4.67 (s, 1H, N—CH₂), 4.65 (s, 1H, N—CH₂), 4.10 (q, J=7.0 Hz, 1H, OCH₂), 1.45 (t, J=7.0 Hz, 1.5H, CH₃). The compounds 7a(Int-1) and 7a(Int-2) were obtained as an orange solid that was used as such in the successive amidation step.

### Example 70

Scheme 6: Preparation of target compounds deriving from intermediates of general formula 7a not included in Scheme 1.

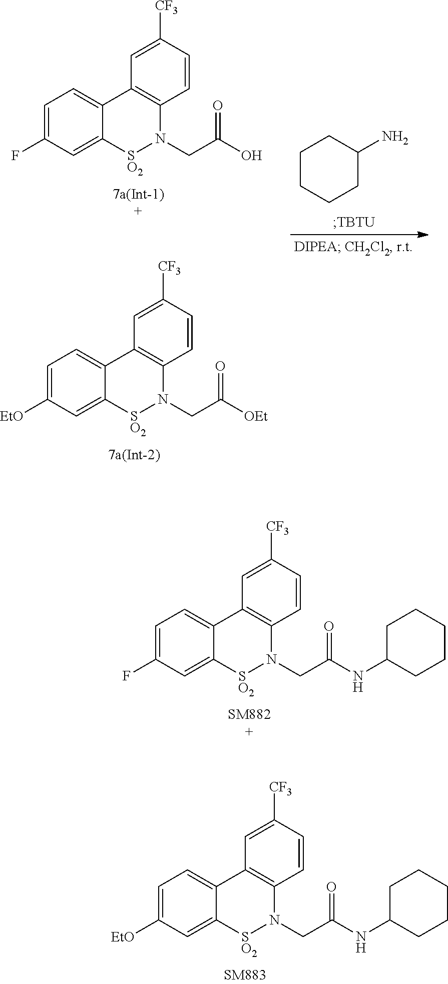

N-cyclohexyl-2-(3-fluoro-5,5-dioxido-9-(trifluoromethyl)-6H-dibenzo[c,e][1,2]thiazin-6-yl)acetamide (SM882) and N-cyclohexyl-2-(3-ethoxy-5,5-dioxido-9-(trifluoromethyl)-6H-dibenzo[c,e][1,2]thiazin-6-yl)acetamide (SM883): A stirred mixture of 7a(Int-1) and 7a(Int-2) (0.50 g, 1.33 mmol), cyclohexylamine (0.18 mL, 1.6 mmol), TBTU (0.55 g, 1.7 mmol), and DIPEA (0.93 mL, 5.33 mmol) in dry CH₂Cl₂ (3 mL) was reacted at room temperature for 1 h. The solvent was then evaporated to dryness and the residue was poured in ice-water obtaining a precipitate that was filtered and the crude was purified by flash chromatography eluting with CH₂Cl₂ obtaining SM882 (R_{f}>) and SM883 (R_{f}<) respectively.

Each compound was further purified by crystallization with EtOH to give: SM882: white solid (0.064 g, 14%), mp 232-233° C. ¹H NMR (400 MHz, CDCl₃): δ1.10-1.20 and 1.30-1.40 (m, each 2H, cyclohexyl CH₂), 1.50-1.70 (m, 4H, cyclohexyl CH₂), 1.80-1.90 (m, 2H, cyclohexyl CH₂), 3.85-3.95 (m, 1H, cyclohexyl CH), 4.55 (s, 1H, NCH₂), 6.45 (d, J=7.5 Hz, 1H, CONH), 7.40 (d, J=8.5 Hz, 1H, Ar—H), 7.55 (dt, J=2.6 and 8.1 Hz, 1H, Ar—H), 7.75-7.85 (m, 2H, Ar—H), 8.10 (dd, J=4.6 and 8.9 Hz, 1H, Ar—H), 8.30 (s, 1H, Ar—H); ¹³C NMR (101 MHz, CDCl₃): δ 24.2, 25.2, 32.4, 48.5, 51.4, 109.8 (d, J_{C-F}=25.4 Hz), 119.8, 120.8 (d, J_{C-F}=22.2 Hz), 122.7 (d, J_{C-F}=3.6 Hz), 123.5, 128.8 (q, J_{C-F}=273.3 Hz), 127.1 (d, J_{C-F}=3.3 Hz), 127.4, 127.7, 128.5 (d, J_{C-F}=8.1 Hz), 135.4 (d, J_{C-F}=7.3 Hz), 140.1, 162.2 (d, J_{C-F}=256.9 Hz), 165.6. HRMS (ESI) m/z [M+H]⁺ calcd. for C₂₁H₂₀F₄N₂O₃S: 457.1210, found: 457.1207.

SM883: white solid (0.069 g, 15%), mp 201-202° C. ¹H NMR (400 MHz, CDCl₃): δ1.10-1.20 (m, 4H, cyclohexyl CH₂), 1.30-1.40 (m, 2H, cyclohexyl CH₂), 1.50 (t, J=6.9 Hz, 3H, OCH₂CH₃), 1.60-1.70 and 1.80-1.90 (m, each 2H, cyclohexyl CH₂), 3.80-3.90 (m, 1H, cyclohexyl CH), 4.20 (q, J=6.9 Hz, 2H, OCH₂CH₃), 4.55 (s, 1H, NCH₂), 6.55 (d, J=7.7 Hz, 1H, CONH), 7.30-7.40 (m, 2H, Ar—H), 7.50 (d, J=2.1 Hz, 1H, Ar—H), 7.70 (d, J=8.6 Hz, 1H, Ar—H), 7.95 (d, J=8.9 Hz, 1H, Ar—H), 8.25 (s, 1H, Ar—H); ¹³C NMR (101 MHz, CDCl₃): δ 14.4, 24.2, 25.2, 32.3, 48.4, 51.2, 64.4, 106.1, 119.3, 121.1, 122.1 (d, J_{C-F}=3.6 Hz), 123.9, 126.1 (d, J_{C-F}=3.3 Hz), 127.5, 134.9, 139.6, 159.6, 166.0. HRMS (ESI) m/z [M+H]⁺ calcd. for C₂₃H₂₅F₃N₂O₄S: 483.1566, found: 483.1565.

### Example 71

N-(1-Ethylpropyl)-2-[3-fluoro-5,5-dioxido-9-(trifluoromethyl)-6H-dibenzo[c,e][1,2]thiazin-6-yl]acetamide (SM884): A stirred mixture of 3-Fluoro-5,5-dioxido-9-(trifluoromethyl)-6H-dibenzo[c,e][1,2]thiazin-6-yl]acetic acid of formula 7a (Example 63; 0.30 g, 0.8 mmol), 3-aminopentane (0.084 g, 0.96 mmol), TBTU (0.33 g, 1.04 mmol), DIPEA (0.56 mL, 3.2 mmol) in CH₂Cl₂ (6 mL) was kept at room temperature for 3 h.

The organic solvent was evaporated and the residue was poured into ice/water and the mixture was acidified with 2N HCl (pH=4) maintaining the mixture under stirring for 40 min. until a precipitated was observed. The precipitate was filtered, dried and crystallized by cyclohexane/EtOAc (3:1 ratio) to obtain SM884 as pinkish solid in 34%: mp 184-185° C. ¹H NMR (400 MHz, CDCl₃): δ 0.80 (t, J=7.4 Hz, 6H, pentyl CH₃), 1.30-1.40 and 1.45-1.55 (m, each 2H, pentyl CH₃), 3.75-3.80 (m, 1H, pentyl CH), 4.50 (s, 1H, NCH₂), 6.25 (d, J=8.6 Hz, 1H, CONH), 7.40 (d, J=8.6 Hz, 1H, Ar—H), 7.50 (dt, J=2.6 and 8.3 Hz, 1H, Ar—H), 7.70-7.75 (m, 2H, Ar—H), 8.10 (dd, J=4.6 and 8.9 Hz, 1H, Ar—H), 8.25 (s, 1H, Ar—H); ¹³C NMR (100 MHz, CDCl₃): δ 9.9, 27.0, 51.4, 52.7, 109.8 (d, J_{C-F}=25.5 Hz), 119.8, 120.9 (d, J_{C-F}=22.3 Hz), 122.7 (d, J_{C-F}=3.5 Hz), 123.4, 126.1 (q, J_{C-F}=273.0 Hz), 127.1 (d, J_{C-F}=3.2 Hz), 127.7, 128.6 (d, J_{C-F}=8.1 Hz), 135.3 (d, J_{C-F}=7.3 Hz), 140.1, 162.2 (d, J_{C-F}=257.0 Hz), 166.4. HRMS (ESI) m/z [M+H]⁺ calcd. for C₂₀H₂₀F₄N₂O₃S: 445.1210, found: 445.1207.

### Example 72

2-[3-Fluoro-5,5-dioxido-9-(trifluoromethyl)-6H-dibenzo[c,e][1,2]thiazin-6-yl]-N-(tetrahydro-2H-pyran-4-yl)acetamide (SM885): following the procedure reported above for compound SM884 and using tetrahydro-2H-pyran-4-amine, the target compound was obtained after crystallization by cyclohexane/EtOAc, in 34% yield as pale pink solid: mp 241-242° C. ¹H NMR (400 MHz, CDCl₃): δ 1.40-1.50, 1.80-1.90, 3.40-3.50, and 3.75-3.85 (m, each 2H, pyran CH₂), 4.00-4.10 (m, 1H, pyran CH), 4.50 (s, 1H, NCH₂), 6.45 (d, J=7.4 Hz, 1H, CONH), 7.40 (d, J=8.6 Hz, 1H, Ar—H), 7.50 (dt, J=2.7 and 8.5 Hz, 1H, Ar—H), 7.65-7.75 (m, 2H, Ar—H), 8.05 (dd, J=4.6 and 8.9 Hz, 1H, Ar—H), 8.25 (s, 1H, Ar—H); ¹³C NMR (100 MHz, CDCl₃): δ 32.4, 46.0, 51.3, 66.2, 109.8 (d, J_{C-F}=25.5 Hz), 119.7, 121.0 (d, J_{C-F}=22.2 Hz), 122.7 (d, J_{C-F}=3.5 Hz), 123.4, 126.1 (q, J_{C-F}=273.0 Hz), 127.2, 127.4 (d, J_{C-F}=3.2 Hz), 127.8, 128.6 (d, J_{C-F}=8.1 Hz), 135.3 (d, J_{C-F}=7.3 Hz), 140.0, 162.2 (d, J_{C-F}=257.2 Hz), 166.0. HRMS (ESI) m/z [M+H]⁺ calcd. for C₂₀H₁₈F₄N₂O₄S: 459.1002, found: 459.1002.

### Example 73

2-[3-Fluoro-5,5-dioxido-9-(trifluoromethyl)-6H-dibenzo[c,e][1,2]thiazin-6-yl]-N-morpholin-4-ylacetamide (SM881): following the procedure reported for compound SM884 and using morpholin-4-amine, the target compound was obtained after crystallization by EtOH, in 34% yield as pale pink solid: mp 276-278° C. Two rotamers were identified by ¹H-NMR and they collapsed to one molecule carrying out experiments at 60° C. ¹H NMR (400 MHz, DMSO-d₆, 25° C.): δ 2.50-2.60, 2.75-2.95, 3.40-3.50, and 3.60-3.80 (m, each 2H, morpholine CH₂), 4.50 and 5.00 (s, each 1H, NCH₂), 7.60-7.75 (m, 2H, Ar—H), 7.75-7.80 and 7.80-7.90 (m, each 1H, Ar—H), 8.45 (dd, J=4.6 and 8.6 Hz, 1H, Ar—H), 8.55 (s, 1H, Ar—H), 8.80 and 9.25 (s, each 0.5H, CONH); ¹³C NMR (100 MHz, DMSO-d₆): δ 32.4, 46.0, 66.2, 110.1 (d, J_{C-F}=25.5 Hz), 119.7, 122.0 (d, J_{C-F}=22.2 Hz), 123.7 (d, J_{C-F}=3.5 Hz), 123.4, 125.1 (q, J_{C-F}=273.0 Hz), 127.2, 127.4 (d, J_{C-F}=3.2 Hz), 127.8, 129.2 (d, J_{C-F}=8.1 Hz), 134.2 (d, J_{C-F}=7.3 Hz), 140.0, 161.2 (d, J_{C-F}=257.2 Hz), 166.0. HRMS (ESI) m/z [M+H]⁺ calcd. for C₁₉H₁₇F₄N₃O₄S: 460.0955, found: 460.0954.

### Example 74

N-(2-chloropyridin-4-yl)-2-[5,5-dioxido-9-(trifluoromethyl)-6H-dibenzo[c,e][1,2]thiazin-6-yl]acetamide (SM880): The title compound was prepared starting from 2-(5,5-dioxido-9-(trifluoromethyl)-6H-dibenzo[c,e][1,2]thiazin-6-yl)acetic acid of formula 7a (Example 75) and following the procedure reported for compound SM884 and using 2-chloro-4-pyridineamine. Title compound was obtained after crystallization by cyclohexane/EtOAc, as pale pink solid in 34% yield: mp 184-185° C. ¹H NMR (400 MHz, CDCl₃): δ 0.80 (t, J=7.4 Hz, 6H, pentyl CH₃), 1.30-1.40 and 1.45-1.55 (m, each 2H, pentyl CH₃), 3.75-3.80 (m, 1H, pentyl CH), 4.50 (s, 1H, NCH₂), 6.25 (d, J=8.6 Hz, 1H, CONH), 7.40 (d, J=8.6 Hz, 1H, Ar—H), 7.50 (dt, J=2.6 and 8.3 Hz, 1H, Ar—H), 7.70-7.75 (m, 2H, Ar—H), 8.10 (dd, J=4.6 and 8.9 Hz, 1H, Ar—H), 8.25 (s, 1H, Ar—H); ¹³C NMR (101 MHz, CDCl₃): δ 9.9, 27.0, 51.4, 52.7, 109.8 (d, J_{C-F}=25.5 Hz), 119.8, 120.9 (d, J_{C-F}=22.3 Hz), 122.7 (d, J_{C-F}=3.5 Hz), 123.4, 126.1 (q, J_{C-F}=273.0 Hz), 127.1 (d, J_{C-F}=3.2 Hz), 127.7, 128.6 (d, J_{C-F}=8.1 Hz), 135.3 (d, J_{C-F}=7.3 Hz), 140.1, 162.2 (d, J_{C-F}=257.0 Hz), 166.4. HRMS (ESI) m/z [M+H]⁺ calcd. for C₂₀H₂₀F₄N₂O₃S: 445.1210, found: 445.1207.

Scheme 7: Preparation of target compounds deriving from intermediates of general formula 7a, not included in Scheme 1.

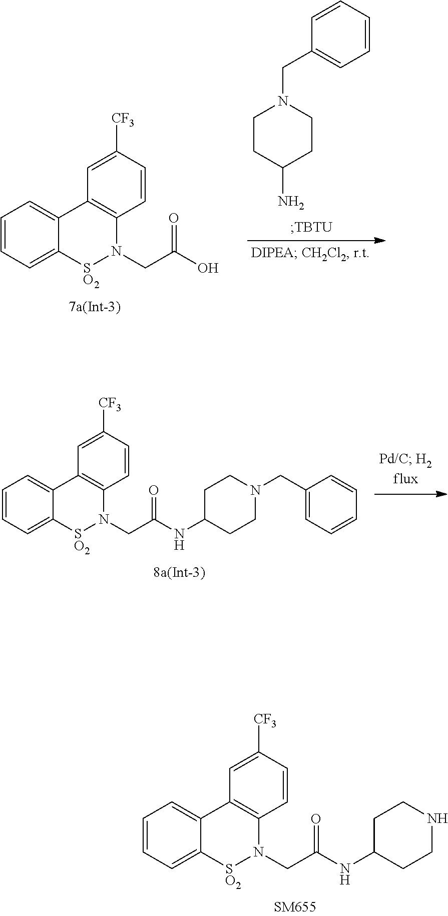

### Example 75

2-(5,5-dioxido-9-(trifluoromethyl)-6H-dibenzo[c,e][1,2]thiazin-6-yl)acetic acid (7a(Int-3)): compound of formula 7a(Int-3) was prepared from ethyl [5,5-dioxido-9-(trifluoromethyl)-6H-dibenzo[c,e][1,2]thiazin-6-yl]acetate according to the procedure reported for a similar compound described in Example 65. The intermediate was obtained as brown solid in 81% yield: ¹H NMR (400 MHz, DMSO-d₆): δ 8.55 (d, J=2.2 Hz, 1H, Ar—H), 8.27 (d, J=8.0 Hz, 1H, Ar—H), 8.00-7.75 (m, 4H, Ar—H), 7.50-7.50 (m, 2H, Ar—H), 4.75 (s, 2H, NCH₂).

### Example 76

N-(1-benzylpiperidin-4-yl)-2-[5,5-dioxido-9-(trifluoromethyl)-6H-dibenzo[c,e][1,2]thiazin-6-yl]acetamide of formula 8a(Int-3): to a solution of 2-(5,5-dioxido-9-(trifluoromethyl)-6H-dibenzo[c,e][1,2]thiazin-6-yl)acetic acid of formula 7a(Int-3) (Example 75; 0.280 g, 0.78 mmol) in dry CH₂Cl₂ (10 mL), N-benzyl-4-aminopiperidine (0.180 g, 0.94 mmol), TBTU (0.376 g, 0.12 mmol), and DIPEA (0.510 mL, 0.31 mmol) were added. The reaction mixture was stirred at room temperature for 4 h and then poured into ice-water and acidified with 2N HCl (pH=2). The mixture was extracted with CH₂Cl₂ (3×30 mL) and the combined organic layers were washed with brine, dried over Na₂SO₄ and evaporated to dryness to give a brown oil which was purified by flash column chromatography (CHCl₃/MeOH 95:5), to afford N-(1-benzylpiperidin-4-yl)-2-[5,5-dioxido-9-(trifluoromethyl)-6H-dibenzo[c,e][1,2]thiazin-6-yl]acetamide as solid in 45% yield: mp ° C. ¹H-NMR (200 MHz, CDCl₃): δ 8.27 (s, 1H, Ar—H), 8.03-7.99 (d, J=7.9 Hz, 2H, Ar—H), 7.82-7.60 (m, 3H, Ar—H), 7.32-7.15 (m, 6H, Ar—H), 6.53 (d, J=7.3 Hz, 1H, NH), 4.54 (s, 2H, benzylic-CH₂), 4.83-4.71 (m, 1H, piperidine-CH), 3.45 (s, 2H, CH₂), 2.67-2.62 (m, 2H, piperidine-CH₂), 2.15-1.80 (m, 6H, piperidine-CH₂×2), 1.47-1.32 (m, 2H, piperidine-CH₂).

### Example 77

2-[5,5-dioxido-9-(trifluoromethyl)-6H-dibenzo[c,e][1,2]thiazin-6-yl]-N-piperidin-4-ylacetamide of general formula 8a (SM655): to a solution of the appropriate compound of formula 8a (0.180 g, 0.34 mmol) in EtOH (20 mL), Pd/C (20% w/w, 0.036 g) was added. The reaction mixture was stirred at room temperature for 7 h under H₂ bubbling. The mixture was filtered over Celite® and the filtrate was evaporated to dryness to give a brown solid which was crystallized by EtOH to afford SM665 as white solid in 27% yield. ¹H NMR (400 MHz, DMSO-d₆): δ 8.55 (s, 1H, Ar—H), 8.38-8.35 (m, 2H, Ar—H), 7.91-7.81 (m, 3H, Ar—H), 7.71 (t, J=7.6 Hz, 1H, Ar—H), 7.62 (d, J=8.6 Hz, 1H, Ar—H), 4.63 (s, 2H, benzylic-CH₂), 3.70-3.59 (m, 1H, piperidine-CH), 3.16-3.13 (m, 2H, piperidine-CH₂), 2.82 (t, J=10.7 Hz, 2H, piperidine-CH₂), 1.77-1.74 (m, 2H, piperidine-CH₂), 1.49-1.37 (m, 2H, piperidine-CH₂).

Scheme 8: Preparation of target compounds deriving from intermediates of general formula 7a, not included in scheme 1.

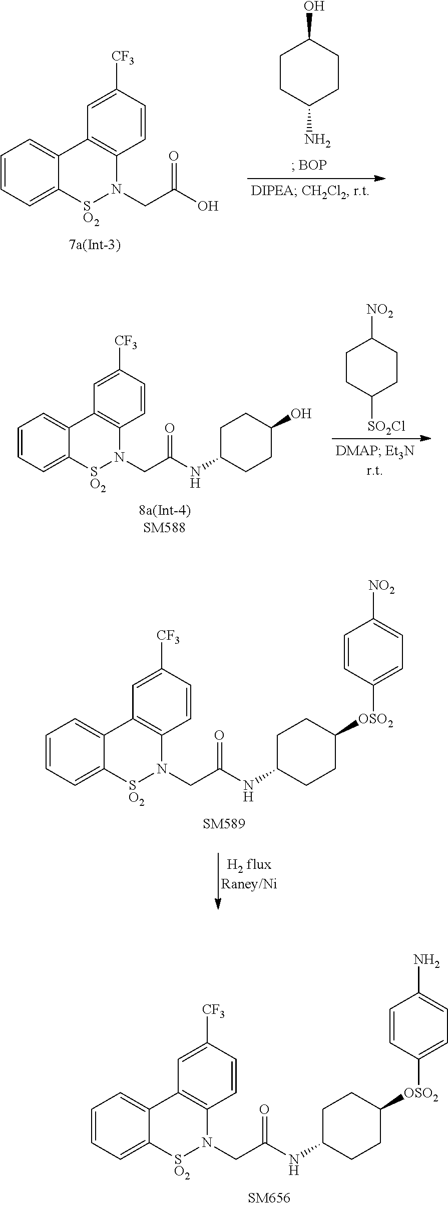

### Example 78

2-[5,5-dioxido-9-(trifluoromethyl)-6H-dibenzo[c,e][1,2]thiazin-6-yl]-N-(trans-4-hydroxycyclohexyl)acetamide of formula 8a(Int-4): to a solution of compound of formula 7a(Int-3) (Example 75; 0.100 g, 0.30 mmol) in dry CH₂Cl₂ (4 mL), trans-4-aminocyclohexanol (0.041 g, 0.36 mmol), BOP (0.199 g, 0.45 mmol), and DIPEA (0.200 mL, 1.2 mmol) were added at 0° C. The reaction mixture was stirred at room temperature for 12 h, then it was concentrated under vacuum and poured into ice-water and acidified with 2N HCl (pH=4). The mixture was extracted with EtOAc (3×20 mL) and the combined organic layers were washed with brine, dried over Na₂SO₄ and evaporated to dryness to give a brown solid which was crystallized by EtOH to afford SM588 8a(Int-4) as a white solid in 88% yield: mp 212-213° C. ¹H-NMR (400 MHz, CDCl₃): δ 8.29 (d, J=1.3 Hz, 1H, Ar—H), 8.03 (d, J=7.3 Hz, 2H, Ar—H), 7.81 (td, J=1.3 and 7.4 Hz, 1H, Ar—H), 7.72 (dd, J=1.6 and 6.3 Hz, 1H, Ar—H), 6.67 (t, J=7.5 Hz, 1H, Ar—H), 7.33 (d, J=7.9 Hz, 1H, Ar—H), 6.52 (d, J=7.6 Hz, 1H, NH), 4.51 (s, 2H, benzyl-CH₂), 3.85-3.77 (m, 1H, cyclohexyl-CH), 3.61-3.46 (m, 1H, cyclohexyl-CH), 1.99-1.89 (m, 4H, cyclohexyl-CH₂×2), 1.46 (s, 1H, OH), 1.42-1.34 (m, 2H, cyclohexyl-CH₂), 1.23-1.16 (m, 2H, cyclohexyl-CH₂).

### Example 79

trans-4-({2-[5,5-dioxido-9-(trifluoromethyl)-6H-dibenzo[c,e][1,2]thiazin-6-yl]acetyl}amino)cyclohexyl 4-nitrobenzenesulfonate of general formula 8a (SM589): to a solution of an appropriate compound SM588 of formula 8a(Int-4) (0.350 g, 0.77 mmol) in dry CH₂Cl₂ (6 mL), 4-nitrobenzenesulfonyl chloride (0.355 g, 1.60 mmol), DMAP (0.094 g, 0.77 mmol), and ET₃N (0.320 mL, 2.30 mmol) were added at 0° C. The reaction mixture was stirred at room temperature for 2 h, then it was concentrated under vacuum and poured into ice-water and acidified with 2N HCl (pH=4). The mixture was extracted with CH₂Cl₂ (3×20 mL) and the combined organic layers were washed with brine, dried over Na₂SO₄ and evaporated to dryness to give a white solid which was crystallized by EtOH to afford SM589 as a white solid in 38% yield: mp 151-152° C. ¹H-NMR (400 MHz, CDCl₃): δ 8.35 (d, J=8.5 Hz, 2H, Ar—H), 8.29 (s, 1H, Ar—H), 8.12-7.93 (m, 4H, Ar—H), 7.81 (t, J=7.0 Hz, 1H, Ar—H), 7.72-7.61 (m, 2H, Ar—H), 7.26 (d, J=8.6 Hz, 1H, Ar—H), 6.64 (d, J=7.5 Hz, 1H, NH), 4.60-4.51 (m, 1H, cyclohexyl-CH), 4.49 (s, 2H, CH₂), 3.91-3.85 (m, 1H, cyclohexyl-CH), 2.01-1.88 (m, 4H, each 2H cyclohexyl-CH₂), 1.68-1.59 (m, 2H, cyclohexyl-CH₂), 1.52 (s, 1H, OH), 1.32-1.10 (m, 2H, cyclohexyl-CH₂).

### Example 80

trans-4-({2-[5,5-dioxido-9-(trifluoromethyl)-6H-dibenzo[c,e][1,2]thiazin-6-yl]acetyl}amino)cyclohexyl 4-aminobenzenesulfonate of formula 8a (SM656): to a solution of compound SM589 (0.400 g, 0.63 mmol) in DMF (30 mL), Raney/Ni (10% w/w, 0.046 g) was added. The reaction mixture was stirred at room temperature for 2 h under H₂ bubbling. The mixture was filtered over Celite©and the filtrate was evaporated to dryness to give a brown solid which was crystallized by EtOH to afford SM656 as brownish solid in 58% yield. ¹H NMR (400 MHz, DMSO-d₆): δ 8.45 (s, 1H, Ar—H), 8.37 (d, J=7.9 Hz, 1H, Ar—H), 8.05 (d, J=7.2 Hz, 1H, Ar—H), 7.90-7.80 (m, 3H, Ar—H and NH), 7.70 (t, J=7.6 Hz, 1H, Ar—H), 7.57 (d, J=8.4 Hz, 1H, Ar—H), 7.44 (d, J=8.8 Hz, 2H, Ar—H), 6.59 (d, J=8.6 Hz, 2H, Ar—H), 6.19 (s, 2H, NH₂), 4.58 (s, 2H, CH₂), 4.11-4.23 (m, 1H, cyclohexyl-CH), 1.69-1.61 (m, 4H, each 2H, cyclohexyl-CH₂), 1.39-1.31 (m, 2H, cyclohexyl-CH₂), 1.17-1.08 (m, 2H, cyclohexyl-CH₂).

### Example 81

2-(3-acetyl-4-hydroxy-1,1-dioxido-2H-1,2-benzothiazin-2-yl)-N-cyclohexylacetamide (12a) (Scheme 4). A mixture of 11a, prepared according to literature, (0.63 g, 2.11 mmol), cyclohexylamine (0.53 mL, 4.66 mmol), TBTU (1.63 g, 5.08 mmol), and Et₃N (4 equiv.) in dry THE was reacted at room temperature for 2 h. The reaction mixture was then poured in ice-water and acidified with 2N HCl (pH=4) obtaining a precipitate that was filtered and dried to give 12a (0.75 g, 94%) as pale-yellow solid. ¹H NMR (400 MHz, DMSO-d₆): δ 0.80-1.20 and 1.40-1.60 (m, each 5H, cyclohexyl CH₂), 2.40 (s, 3H, CH₃), 4.00 (s, 2H, NCH₂), 7.75-7.85 (m, 4H, Ar—H and CONH), 7.95-8.10 (m, 1H, Ar—H), 15.20 (bs, 1H, OH).

### Example 82

N-cyclohexyl-2-(3-methyl-5,5-dioxidopyrazolo[4,3-c][1,2]benzothiazin-4 (1H)-yl)acetamide (SM879). The mixture of 12a (0.30 g, 0.79 mmol) and hydrazine monohydrate (0.19 mL, 3.96 mmol) was reacted at 60° C. for 1 h. After cooling, the reaction mixture was poured in ice-water and acidified with 2N HCl (pH=4), yielding a precipitate that was filtered and purified by flash chromatography eluting with CH₂Cl₂:MeOH 97:3 followed by crystallization by EtOH to afford SM879 (0.08 g, 54%) as a white solid. ¹H NMR (400 MHz, CDCl₃) δ 1.10-1.20 and 1.25-1.45 (m, each 2H, cyclohexyl CH₂), 1.50-1.75 (m, 4H, cyclohexyl CH₂), 2.80-2.90 (m, 2H, cyclohexyl CH₂), 2.30 (s, 1H, CH₃), 3.75 (m, 1H, cyclohexyl CH), 4.05 (s, 2H, NCH₂), 6.50 (d, J=8.1 Hz, 1H, NH), 7.55 (dt, J=1.2 and 7.8 Hz, 1H, Ar—CH), 7.70 (dt, J=1.2 and 7.7 Hz, 1H, Ar—CH), 7.80 (dd, J=0.9 and 7.8 Hz, 1H, Ar—CH), 7.95 (d, J=7.3 Hz, 1H, Ar—CH), 10.50 (bs, 1H, CONH). HRMS (ESI) m/z [M+H]⁺ calcd for C₂₄H₃₁N₃O₄S: 375.1460, found: 375.1485; LC-MS: ret. time 4.109 min.

### Example 83

### Scheme 9: Synthetic Procedure for the Preparation of Target Compound SM886.

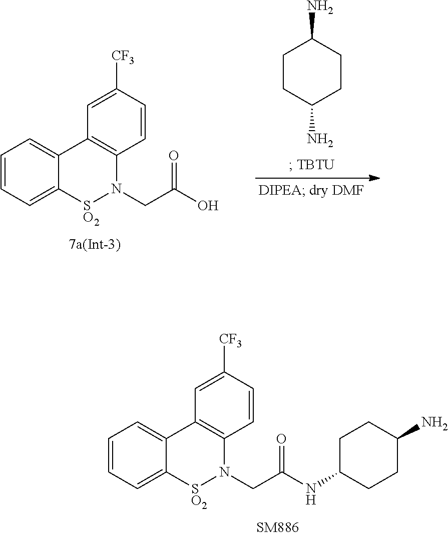 [SMILES: O=C(O)CN1c2ccc(C(F)(F)F)cc2-c2ccccc2S1(=O)=O ; N[C@H]1CC[C@H](N)CC1 ; N[C@H]1CC[C@H](NC(=O)CN2c3ccc(C(F)(F)F)cc3-c3ccccc3S2(=O)=O)CC1]

N-(4-aminocyclohexyl)-2-[5,5-dioxido-9-(trifluoromethyl)-6H-dibenzo[c,e][1,2]thiazin-6-yl]acetamide (SM886). A stirred mixture of [5,5-dioxido-9-(trifluoromethyl)-6H-dibenzo[c,e][1,2]thiazin-6-yl]acetic acid of formula 7a(Int-3) (0.10 g, 0.28 mmol), trans-1,4-diaminocyclohexane (0.32 g, 2.80 mmol), TBTU (0.12 g, 0.36 mmol), DIPEA (0.19 mL, 1.12 mmol) in dry DMF (3 mL) was kept at room temperature for 3 h. The reaction mixture was poured into ice/water and extracted with CH₂Cl₂ (×3). The combined organic layers were washed with brine, dried over Na₂SO₄ and evaporated to dryness to give a brown oil. After purification by trituration with Et₂O, the title compound was obtained as a yellow solid in 16% yield: m.p. 212-214° C. ¹H NMR (400 MHz, MeOD): δ 1.16-1.29 (m, 4H, CH₂×2), 1.87-1.89 (m, 4H, CH₂×2), 2.60-2.63 (m, 1H, CH), 3.49-3.54 (m, 1H, CH), 4.66 (s, 2H, NCH₂), 7.57 (d, J=8.6 Hz, 1H, Ar—H), 7.73 (t, J=7.5 Hz, 1H, Ar—H), 7.82-7.89 (m, 2H, Ar—H), 7.99 (d, J=7.8 Hz, 1H, Ar—H), 8.24 (d, J=8.0 Hz, 1H, Ar—H), 8.49 (s, 1H, Ar—H). HRMS (ESI) m/z [M+H]⁺ calcd. for C₂₁H₂₂F₃N₃O₃S: 454.1412, found: 454.14162.

### Example 84

Scheme 10: Synthetic procedure for the preparation of target compound SM887.

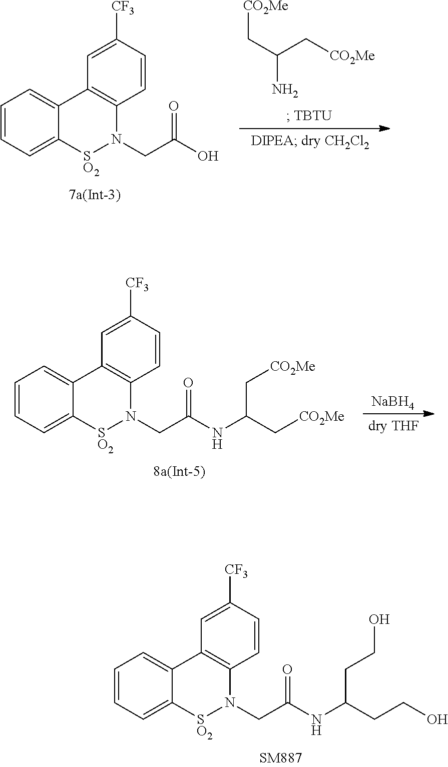 [SMILES: O=C(O)CN1c2ccc(C(F)(F)F)cc2-c2ccccc2S1(=O)=O ; COC(=O)CC(N)CC(=O)OC ; COC(=O)CC(CC(=O)OC)NC(=O)CN1c2ccc(C(F)(F)F)cc2-c2ccccc2S1(=O)=O ; "NaBH4" ; O=C(CN1c2ccc(C(F)(F)F)cc2-c2ccccc2S1(=O)=O)NC(CCO)CCO]

Dimethyl 3-({[5,5-dioxido-9-(trifluoromethyl)-6H-dibenzo[c,e][1,2]thiazin-6-yl]acetyl}amino)pentanedioate (8a(Int-5)). A stirred mixture of [5,5-dioxido-9-(trifluoromethyl)-6H-dibenzo[c,e][1,2]thiazin-6-yl]acetic acid of formula 7a(Int3) (0.33 g, 0.92 mmol), dimethyl 3-aminopentanedioate (0.19 g, 1.11 mmol), TBTU (0.38 g, 1.19 mmol), DIPEA (0.64 mL, 3.68 mmol) in CH₂Cl₂ (10 mL) was kept at room temperature for 2 h. The organic solvent was evaporated, and the residue was poured into ice/water and extracted with EtOAc (×3). The combined organic layers were washed with brine, dried over Na₂SO₄ and evaporated to dryness to give a brown oil. After purification by flash column chromatography, eluting with CHCl₃/MeOH (98:2), the title compound was obtained as a white solid in 25% yield: m.p. 126-128° C. ¹H NMR (400 MHz, CDCl₃): δ 2.37-2.44 (m, 4H, CH₂×2), 3.58 (s, 6H, OCH₃×2), 4.52 (s, 2H, NCH₂), 4.59-4.64 (m, 1H, CH), 7.17 (d, J=8.4 Hz, 1H, NH), 7.40 (d, J=8.5 Hz, 1H, H-7), 7.65 (t, J=7.8 Hz, 1H, H-3), 7.72 (d, J=8.5 Hz, 1H, H-8), 7.79 (td, J=1.0 and 7.4 Hz, 1H, H-2), 8.02 (d, J=8.1 Hz, 2H, H-1 and H-4), 8.27 (s, 1H, H-10).

2-[5,5-dioxido-9-(trifluoromethyl)-6H-dibenzo[c,e][1,2]thiazin-6-yl]-N-[3-hydroxy-1-(2-hydroxyethyl)propyl]acetamide (SM887). A stirred mixture of dimethyl 3-({[5,5-dioxido-9-(trifluoromethyl)-6H-dibenzo[c,e][1,2]thiazin-6-yl]acetyl}amino)pentanedioate of formula 8a(Int-5) (0.45 g, 0.87 mmol) and NaBH₄ (1.32 g, 35.97 mmol) in dry THE (20 mL) was stirred at reflux for 16 h. Then, the reaction mixture was cooled up to 0° C. and MeOH (15 mL) was added to quench the excess of NaBH₄. The organic solvent was evaporated and the residue was poured into ice/water and extracted with EtOAc (×3). The combined organic layers were washed with brine, dried over Na₂SO₄ and evaporated to dryness to give a yellow oil. After purification by flash column chromatography, eluting with CHCl₃/MeOH (98:2), the title compound was obtained as a white solid in 28% yield: m.p. 136-138° C. ¹H NMR (400 MHz, DMSO-d₆): δ 1.45-1.58 (m, 4H, CH₂×2), 3.29-3.39 (m, 4H, CH₂×2), 3.76-3.78 (m, 1H, CH), 4.30 (t, J=5.1 Hz, 2H, OH×2), 4.66 (s, 2H, NCH₂), 7.65 (d, J=8.5 Hz, 1H, H-7), 7.76 (t, J=7.5 Hz, 1H, H-3), 7.86-7.93 (m, 2H, Ar—H), 7.97 (d, J=7.2 Hz, 1H, Ar—H), 8.01 (d, J=8.6 Hz, 1H, NH), 8.43 (d, J=7.9 Hz, 1H, Ar—H), 8.60 (s, 1H, H-10). ¹³C NMR (101 MHz, DMSO-d₆): δ 38.0, 44.1, 49.7, 58.2, 121.6, 121.7, 123.3, 124.4 (q, J_{C-F}=268.4 Hz), 124.7, 125.6 (q, J_{C-F}=32.6 Hz), 127.1, 127.2, 129.9, 130.9, 133.2, 135.0, 141.9, 166.2. HRMS (ESI) m/z [M+K]⁺ calcd. for C₂₀H₂₁F₃N₂05S: 497.0760, found: 497.0756.

### Example 85

### Scheme 11: Synthetic Procedure for the Preparation of Target Compound SM888.

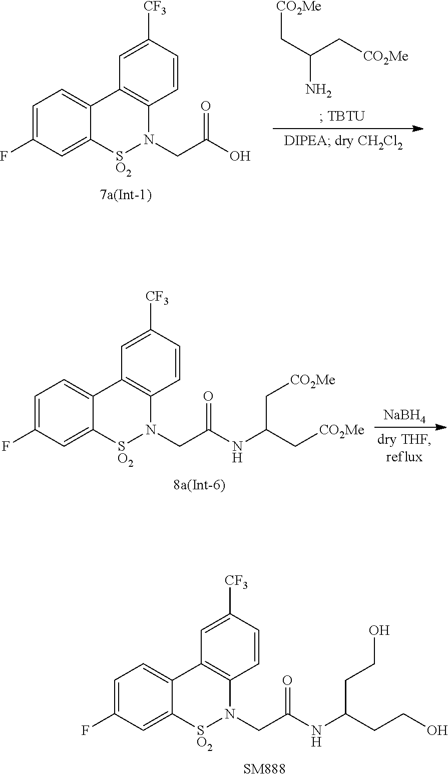 [SMILES: O=C(O)CN1c2ccc(C(F)(F)F)cc2-c2ccc(F)cc2S1(=O)=O ; COC(=O)CC(N)CC(=O)OC ; COC(=O)CC(CC(=O)OC)NC(=O)CN1c2ccc(C(F)(F)F)cc2-c2ccc(F)cc2S1(=O)=O ; "NaBH4" ; O=C(CN1c2ccc(C(F)(F)F)cc2-c2ccc(F)cc2S1(=O)=O)NC(CCO)CCO]

Alternative procedure for the synthesis of [3-Fluoro-5,5-dioxido-9-(trifluoromethyl)-6H-dibenzo[c,e][1,2]thiazin-6-yl]acetic acid (7a(Int-1)). A stirred mixture of compound of formula 6a(Int-1) (1.25 g, 3.10 mmol) in aqueous 1N LiOH (15.5 mL, 15.5 mmol) and dioxane (30 mL) was kept at room temperature for 30 min. The reaction mixture was poured into ice-water and acidified with 2N HCl (pH=2). The formed precipitate was filtered off and dried to give the title compound in 98% yield: m.p. 100-102° C. ¹H NMR (400 MHz, CDCl₃): δ 4.72 (s, 2H, NCH₂), 7.33 (d, J=8.4 Hz, 1H, Ar—H), 7.45 (td, J=2.5 and 8.1 Hz, 1H, H-2), 7.65-7.72 (m, 2H, Ar—H), 7.98 (dd, J=4.4 and 8.6 Hz, 1H, H-1), 8.21 (s, 1H, H-10).

Dimethyl 3-({[3-fluoro-5,5-dioxido-9-(trifluoromethyl)-6H-dibenzo[c,e][1,2]thiazin-6-yl]acetyl}amino)pentanedioate (8a(Int-6)). A stirred mixture of [3-fluoro-5,5-dioxido-9-(trifluoromethyl)-6H-dibenzo[c,e][1,2]thiazin-6-yl]acetic acid of formula 7a(Int-1) (0.71 g, 1.9 mmol), dimethyl 3-aminopentanedioate (0.40 g, 2.28 mmol), TBTU (0.79 g, 2.47 mmol), DIPEA (1.32 mL, 7.6 mmol) in CH₂Cl₂ (30 mL) was kept at room temperature for 2 h. The organic solvent was evaporated and the residue was poured into ice/water and the mixture was acidified with 2N HCl (pH=4) maintaining the mixture under stirring for 10 min. until a precipitated was observed. The precipitate was filtered to give the title compound as a white solid in 87%: m.p. 153-155° C. ¹H NMR (400 MHz, CDCl₃): δ 2.60-2.69 (m, 4H, CH₂×2), 3.89 (s, 6H, OCH₃×2), 4.57 (s, 2H, NCH₂), 4.64-4.66 (m, 1H, CH), 7.14 (d, J=8.5 Hz, 1H, NH), 7.48 (d, J=8.4 Hz, 1H, Ar—H), 7.54 (td, J=2.3 and 8.4 Hz, 1H, Ar—H), 7.75-7.77 (m, 2H, Ar—H), 8.07 (dd, J=4.7 and 8.9 Hz, 1H, Ar—H), 8.26 (s, 1H, Ar—H). 2-[3-Fluoro-5,5-dioxido-9-(trifluoromethyl)-6H-dibenzo[c,e][1,2]thiazin-6-yl]-N-[3-hydroxy-1-(2-hydroxyethyl)propyl]acetamide (SM888). A stirred mixture of dimethyl 3-({[3-fluoro-5,5-dioxido-9-(trifluoromethyl)-6H-dibenzo[c,e][1,2]thiazin-6-yl]acetyl}amino)pentanedioate of formula 8a(Int-6) (0.45 g, 0.85 mmol) and NaBH₄ (1.28 g, 33.81 mmol) in dry THE (15 mL) was stirred at reflux for 30 h. Then, the reaction mixture was cooled up to 0° C. and MeOH (15 mL) was added to quench the excess of NaBH₄. The organic solvent was evaporated and the residue was poured into ice/water and extracted with EtOAc (×3). The combined organic layers were washed with brine, dried over Na₂SO₄ and evaporated to dryness to give a yellow oil. After purification by flash column chromatography, eluting with cyclohexane/EtOAc (70:30), the title compound was obtained as a white solid in 9% yield: m.p. 136-138° C. ¹H NMR (400 MHz, DMSO-d₆): δ 1.43-1.58 (m, 4H, CH₂×2), 3.27-3.34 (m, 4H, CH₂×2), 3.74-3.76 (m, 1H, CH), 4.30 (t, J=5.1 Hz, 2H, OH×2), 4.66 (s, 2H, NCH₂), 7.69 (d, J=8.5 Hz, 1H, H-7), 7.76 (td, J=2.7 and 8.7 Hz, 1H, H-2), 7.84 (dd, J=2.7 and 8.6 Hz, 1H, H-4), 7.91 (dd, J=1.6 and 8.5 Hz, 1H, H-8), 8.02 (d, J=8.6 Hz, 1H, NH), 8.51 (dd, J=4.4 and 8.3 Hz, 1H, H-1), 8.60 (s, 1H, H-10). ¹³C NMR (101 MHz, DMSO-d₆): δ 38.2, 44.3, 50.7, 58.3, 109.1 (d, J_{C-F}=25.5 Hz), 120.8 (d, J_{C-F}=22.1 Hz), 122.5, 123.5, 124.5 (q, J_{C-F}=274.0 Hz), 124.7, 126.1 (q, J_{C-F}=33.2 Hz), 127.2, 127.9, 130.7 (d, J_{C-F}=8.4 Hz), 136.7 (d, J_{C-F}=7.5 Hz), 141.7, 162.3 (d, J_{C-F}=252.8 Hz), 166.4. HRMS (ESI) m/z [M+Na]⁺ calcd. for C₂₀H₂₀F₄N₂O₅S: 499.09267, found: 499.09354.

### Example 86

N-{1-[(dimethylamino)methyl]propyl}-2-[3-fluoro-5,5-dioxido-9-(trifluoromethyl)-6H-dibenzo[c,e][1,2]thiazin-6-yl]acetamide (SM889). A stirred mixture of [3-fluoro-5,5-dioxido-9-(trifluoromethyl)-6H-dibenzo[c,e][1,2]thiazin-6-yl]acetic acid of formula 7a(Int-1) (0.30 g, 0.8 mmol), (2-aminobutyl)dimethylamine (0.13 mL, 0.96 mmol), TBTU (0.33 g, 1.04 mmol), DIPEA (0.56 mL, 3.2 mmol) in CH₂Cl₂ (30 mL) was kept at room temperature for 1 h. The organic solvent was evaporated, and the residue was poured into ice/water and extracted with EtOAc (×3). The combined organic layers were washed with brine, dried over Na₂SO₄ and evaporated to dryness to give a brown oil. After purification by flash column chromatography, eluting with CHCl₃/MeOH (95:5), the title compound was obtained as a light brown solid in 17% yield: m.p. 167-169° C. ¹H NMR (400 MHz, CDCl₃): δ 0.88 (t, J=7.4 Hz, 3H, CH₂CH₃), 1.46-1.49 (m, 1H, CHCH₂CH₃×1/2), 1.60-1.62 (m, 1H, CHCH₂CH₃×1/2), 2.21-2.23 (m, 1H, CHCH₂N×1/2), 2.26-2.29 (m, 1H, CHCH₂N×1/2), 3.89-3.93 (m, CH, 1H), 4.46 (d, J=17.5 Hz, 1H, NCH₂×1/2), 4.70 (d, J=17.5 Hz, 1H, NCH₂×1/2), 6.46 (d, J=6.0 Hz, 1H, NH), 7.51-7.56 (m, 1H, H-2), 7.60 (d, J=8.6 Hz, 1H, H-7), 7.73-7.77 (m, 2H, H-4 and H-8), 8.07 (dd, J=4.5 and 8.8 Hz, 1H, H-1), 8.26 (s, 1H, H-10). ¹³C NMR (101 MHz, CDCl₃): δ 9.9, 25.9, 45.7, 49.3, 51.5, 62.5, 110.0 (d, J_{C-F}=25.3 Hz), 120.8, 120.9 (d, J_{C-F}=21.2 Hz), 122.7 (d, J_{C-F}=3.0 Hz), 123.7 (q, J_{C-F}=273.7 Hz), 123.9, 127.2, 127.7 (d, J_{C-F}=3.0 Hz), 127.8 (q, J_{C-F}=33.3 Hz), 128.7 (d, J_{C-F}=8.0 Hz), 135.9 (d, J_{C-F}=7.1 Hz), 140.5, 162.4 (d, J_{C-F}=256.5 Hz), 166.8. HRMS (ESI) m/z [M+H]⁺ calcd. for C₂₁H₂₃F₄N₃O₃S: 474.1474, found: 474.14908.

### Example 87

Scheme 12: Synthetic Procedure for the Preparation of the Intermediate of Formula 7a(Int-4).

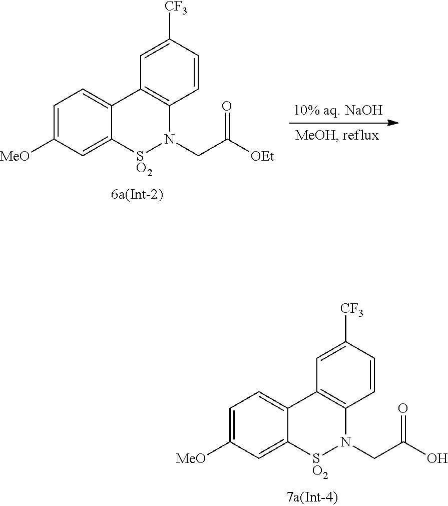 [SMILES: CCOC(=O)CN1c2ccc(C(F)(F)F)cc2-c2ccc(OC)cc2S1(=O)=O ; COc1ccc2c(c1)S(=O)(=O)N(CC(=O)O)c1ccc(C(F)(F)F)cc1-2]

[3-Methoxy-5,5-dioxido-9-(trifluoromethyl)-6H-dibenzo[c,e][1,2]thiazin-6-yl]acetic acid (7a(Int-4)). A stirred mixture of compound of formula 6a(Int-2) (0.34 g, 0.76 mmol) in aqueous 10% NaOH (3 mL) and MeOH (3 mL) was stirred at reflux for 1 h. The reaction mixture was poured into ice/water and acidified with 2N HCl (pH=2). The formed precipitate was filtered off and dried to give the title compound in 46% yield; m.p. 184-186° C. ¹H NMR (400 MHz, DMSO-d₆): δ 3.47 (s, 3H, OCH₃), 4.27 (s, 2H, NCH₂), 7.38-7.41 (m, 2H, Ar—H), 7.55-7.58 (m, 1H, Ar—H), 7.76 (d, J=7.3 Hz, 1H, Ar—H), 8.31 (d, J=8.7 Hz, 1H, Ar—H), 8.45 (s, 1H, Ar—H).

Scheme 13: Synthetic Procedure for the Preparation of the Intermediates of Formula 8a(Int-7), 8a(Int-8) and 8a(Int-9).

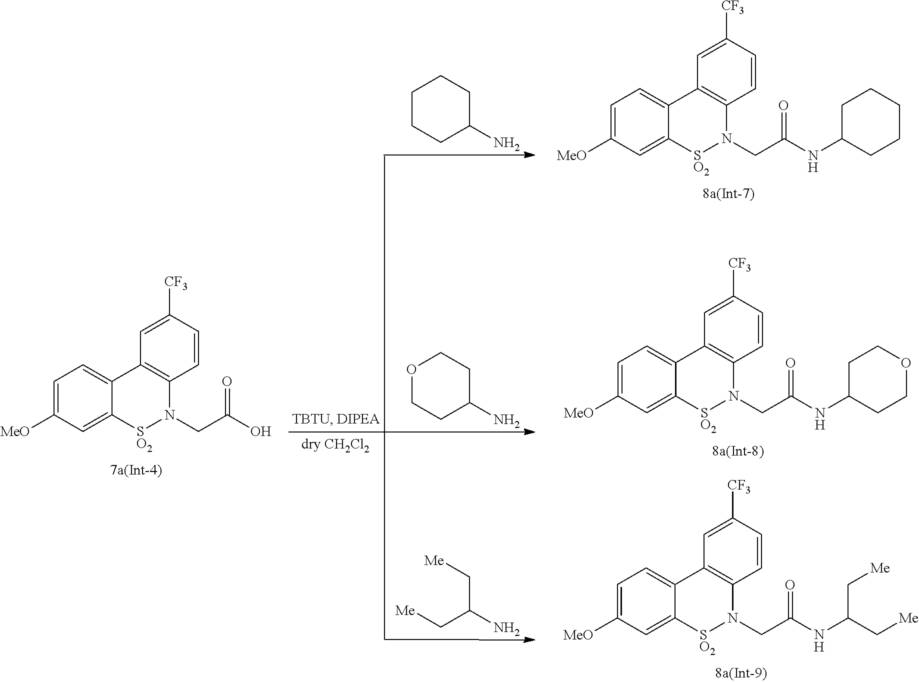

### Example 88

N-cyclohexyl-2-[3-methoxy-5,5-dioxido-9-(trifluoromethyl)-6H-dibenzo[c,e][1,2]thiazin-6-yl]acetamide (8a(Int-7)). A stirred mixture of 3-methoxy-9-(trifluoromethyl)-6H-dibenzo[c,e][1,2]thiazine 5,5-dioxide of formula 7a(Int-4) (0.19 g, 0.48 mmol), cyclohexylamine (0.07 mL, 0.57 mmol), TBTU (0.20 g, 0.62 mmol), DIPEA (0.25 mL, 1.91 mmol) in dry CH₂Cl₂ (10 mL) was kept at room temperature for 2 h. The organic solvent was evaporated, and the residue was poured into ice/water. The obtained precipitate was filtered to give the title compound as a white solid in 58% yield: m.p. 184-185° C. ¹H NMR (400 MHz, CDCl₃): δ 1.14-1.22 (m, 4H, CH₂×2), 1.32-1.41 (m, 2H, CH₂), 1.63-1.66 (m, 2H, CH₂), 1.86-1.89 (m, 2H, CH₂), 3.87-3.89 (m, 1H, CH), 3.98 (s, 3H, OCH₃), 4.55 (s, 2H, NCH₂), 6.58 (d, J=7.3 Hz, 1H, NH), 7.34-7.38 (m, 2H, H-1 and H-2), 7.52 (d, J=2.4 Hz, 1H, H-4), 7.70 (d, J=8.6 Hz, 1H, H-8), 7.97 (d, J=8.8 Hz, 1H, H-7), 8.24 (s, 1H, H-10).

### Example 89

2-[3-Methoxy-5,5-dioxido-9-(trifluoromethyl)-6H-dibenzo[c,e][1,2]thiazin-6-yl]-N-(tetrahydro-2H-pyran-4-yl)acetamide (8a(Int-8)). A stirred mixture of 3-methoxy-9-(trifluoromethyl)-6H-dibenzo[c,e][1,2]thiazine 5,5-dioxide of formula 7a(Int-4) (0.45 g, 0.96 mmol), 4-aminotetrahydropyran (0.12 mL, 1.15 mmol), TBTU (0.40 g, 1.25 mmol), DIPEA (0.67 mL, 3.84 mmol) in dry CH₂Cl₂ (10 mL) was kept at room temperature for 2 h. The organic solvent was evaporated, and the residue was poured into ice/water. The obtained precipitate was filtered to give the title compound as a white solid in 55% yield: m.p. 118-120° C. ¹H NMR (400 MHz, CDCl₃): δ 1.07-1.24 (m, 2H, CH₂), 1.29-1.40 (m, 1H, CH₂×1/2), 1.59-1.65 (m, 1H, CH₂×1/2), 3.23-3.42 (m, 3H, CH₂×1/2 and CH₂), 3.60-3.69 (m, 1H, CH), 3.75-3.80 (m, 1H, CH₂×1/2), 3.93 (s, 3H, OCH₃), 4.63 (s, 2H, NCH₂), 7.38-7.40 (m, 2H, Ar—H and CONH), 7.56-7.63 (m, 1H, Ar—H), 7.78-7.84 (m, 1H, Ar—H), 8.21-8.25 (m, 1H, Ar—H), 8.35-8.33 (m, 1H, Ar—H), 8.50 (s, 1H, Ar—H).

### Example 90

N-(1-ethylpropyl)-2-[3-methoxy-5,5-dioxido-9-(trifluoromethyl)-6H-dibenzo[c,e][1,2]thiazin-6-yl]acetamide (8a(Int-9)). A stirred mixture of 3-methoxy-9-(trifluoromethyl)-6H-dibenzo[c,e][1,2]thiazine 5,5-dioxide of formula 7a(Int-4) (0.45 g, 0.96 mmol), 3-aminopentane (0.13 mL, 1.15 mmol), TBTU (0.40 g, 1.25 mmol), DIPEA (0.67 mL, 3.84 mmol) in dry CH₂Cl₂ (10 mL) was kept at room temperature for 2 h. The organic solvent was evaporated, and the residue was poured into ice/water. The obtained precipitate was filtered to give the title compound as a white solid in 55% yield: m.p. 151-153° C. ¹H NMR (400 MHz, CDCl₃): δ 0.73 (t, J=6.7 Hz, 6H, CH₃×2), 1.24-1.29 (m, 2H, CH₂), 1.38-1.44 (m, 2H, CH₂), 3.39-3.43 (m, 1H, CH), 3.93 (s, 3H, OCH₃), 4.65 (s, 2H, NCH₂), 7.39-7.42 (m, 2H, Ar—H and CONH), 7.63 (d, J=8.1 Hz, 1H, Ar—H), 7.83 (d, J=8.2 Hz, 1H, Ar—H), 7.89 (d, J=8.2 Hz, 1H, Ar—H), 8.35 (d, J=8.3 Hz, 1H, Ar—H), 8.50 (s, 1H, Ar—H).

### Scheme 14: Synthetic Procedure for the Preparation of Target Compound SM890.

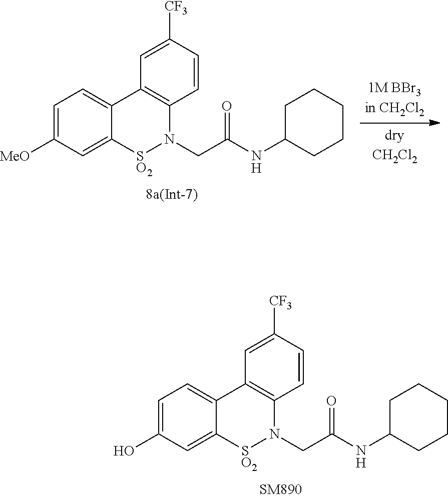 [SMILES: COc1ccc2c(c1)S(=O)(=O)N(CC(=O)NC1CCCCC1)c1ccc(C(F)(F)F)cc1-2 ; O=C(CN1c2ccc(C(F)(F)F)cc2-c2ccc(O)cc2S1(=O)=O)NC1CCCCC1]

N-cyclohexyl-2-[3-hydroxy-5,5-dioxido-9-(trifluoromethyl)-6H-dibenzo[c,e][1,2]thiazin-6-yl]acetamide (SM890). To a solution of N-cyclohexyl-2-[3-methoxy-5,5-dioxido-9-(trifluoromethyl)-6H-dibenzo[c,e][1,2]thiazin-6-yl]acetamide 8a(Int-7) (0.13 g, 0.28 mmol) in dry CH₂Cl₂ (8 mL), 1M BBr₃ in dry CH₂Cl₂ (0.84 mL, 0.84 mmol) was added dropwise at 0° C. and then, the reaction mixture was kept at 10° C. for 2 h.

The mixture was poured into ice/water and extracted in EtOAc (×3). The combined organic layers were washed with brine, dried over Na₂SO₄ and evaporated to dryness to give a brown solid. After purification by flash column chromatography, eluting with CHCl₃/MeOH (99:1), the title compound was obtained as a little brown solid in 24% yield: m.p. 236-240° C. ¹H NMR (400 MHz, DMSO-d₆): δ 1.13-1.20 (m, 6H, CH₂×3), 1.51-1.56 (m, 1H, CH), 1.65-1.67 (m, 4H, CH₂×2), 4.60 (s, 2H, NCH₂), 7.22-7.25 (m, 2H, Ar—H), 7.57 (d, J=8.3 Hz, 1H, Ar—H), 7.80 (d, J=7.9 Hz, 1H, NH), 8.06-8.09 (m, 1H, Ar—H), 8.22 (d, J=8.3 Hz, 1H, Ar—H), 8.43 (s, 1H, Ar—H), 10.78 (bs, 1H, OH). ¹³C NMR (101 MHz, DMSO-d₆): δ 24.7, 25.5, 32.6, 48.1, 49.8, 107.1, 120.7, 121.6, 121.7, 122.1 (2C), 124.5 (q, J_{C-F}=272.9 Hz), 125.4 (q, J_{C-F}=32.8 Hz), 125.6, 129.2, 136.2, 140.8, 158.9, 165.5. HRMS (ESI) m/z [M+Na]⁺ calcd. for C₂₁H₂₁F₃N₂04S: 477.1071, found: 477.10749.

### Scheme 15: Synthetic Procedure for the Preparation of Target Compound SM891.

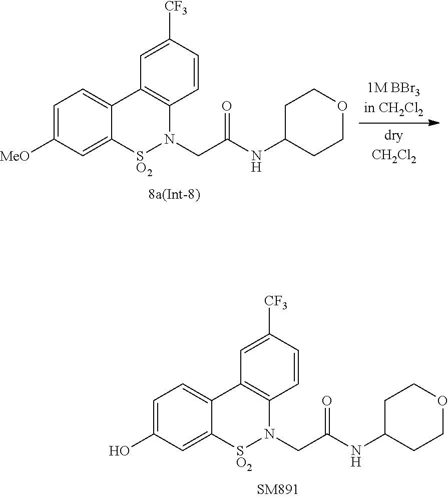 [SMILES: COc1ccc2c(c1)S(=O)(=O)N(CC(=O)NC1CCOCC1)c1ccc(C(F)(F)F)cc1-2 ; O=C(CN1c2ccc(C(F)(F)F)cc2-c2ccc(O)cc2S1(=O)=O)NC1CCOCC1]

2-[3-Hydroxy-5,5-dioxido-9-(trifluoromethyl)-6H-dibenzo[c,e][1,2]thiazin-6-yl]-N-(tetrahydro-2H-pyran-4-yl)acetamide (SM891). To a solution of 2-[3-methoxy-5,5-dioxido-9-(trifluoromethyl)-6H-dibenzo[c,e][1,2]thiazin-6-yl]-N-(tetrahydro-2H-pyran-4-yl)acetamide 8a(Int-8) (0.25 g, 0.52 mmol) in dry CH₂Cl₂ (6 mL), 1M BBr₃ in dry CH₂Cl₂ (2.34 mL, 2.34 mmol) was added dropwise at 0° C. and then, the reaction mixture was kept at 10° C. for 24 h. The mixture was poured into ice/water and extracted in EtOAc (×3). The combined organic layers were washed with brine, dried over Na₂SO₄ and evaporated to dryness to give a brown solid. After purification by flash column chromatography, eluting with CHCl₃/MeOH (98:2), the title compound was obtained as a little brown solid in 6% yield: m.p. 226-228° C. ¹H NMR (400 MHz, DMSO-d₆): δ 1.30-1.39 (m, 2H, CH₂), 1.62-1.65 (m, 2H, CH₂), 3.26-3.32 (m, 2H, CH₂O), 3.63-3.67 (m, 1H, CH), 3.77-3.80 (m, 2H, CH₂O), 4.61 (s, 2H, NCH₂), 7.21-7.24 (m, 2H, Ar—H), 7.58 (d, J=8.3 Hz, 1H, Ar—H), 7.81 (d, J=8.6 Hz, 1H, Ar—H), 8.21-8.24 (m, 2H, Ar—H and NH), 8.44 (s, 1H, Ar—H), 10.70 (bs, 1H, OH).

### Scheme 16: Synthetic Procedure for the Preparation of Target Compound SM892.

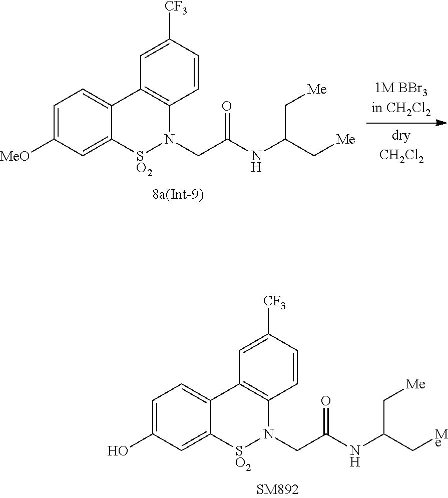 [SMILES: CCC(CC)NC(=O)CN1c2ccc(C(F)(F)F)cc2-c2ccc(OC)cc2S1(=O)=O ; CCC(CC)NC(=O)CN1c2ccc(C(F)(F)F)cc2-c2ccc(O)cc2S1(=O)=O]

N-(1-ethylpropyl)-2-[3-hydroxy-5,5-dioxido-9-(trifluoromethyl)-6H-dibenzo[c,e][1,2]thiazin-6-yl]acetamide (SM892). To a solution of N-(1-ethylpropyl)-2-[3-methoxy-5,5-dioxido-9-(trifluoromethyl)-6H-dibenzo[c,e][1,2]thiazin-6-yl]acetamide 8a(Int-9) (0.23 g, 0.72 mmol) in dry CH₂Cl₂ (6 mL), 1M BBr₃ in dry CH₂Cl₂ (2.16 mL, 2.16 mmol) was added dropwise at 0° C. and then, the reaction mixture was kept at 10° C. for 2 h. The mixture was poured into ice/water and extracted in EtOAc (×3). The combined organic layers were washed with brine, dried over Na₂SO₄ and evaporated to dryness to give a white solid. After purification by flash column chromatography, eluting with CHCl₃/MeOH (98:2), the title compound was obtained as a white solid in 35% yield: m.p. 216-218° C. ¹H NMR (400 MHz, CDCl₃): δ 0.74 (t, J=7.2 Hz, 6H, CH₃×2), 1.20-1.28 (m, 2H, CH₂), 1.36-1.43 (m, 2H, CH₂), 3.37-3.43 (m, 1H, CH), 4.62 (s, 2H, NCH₂), 7.19-7.24 (m, 2H, Ar—H and CONH), 7.59 (d, J=8.4 Hz, 1H, Ar—H), 7.79 (d, J=7.5 Hz, 1H, Ar—H), 7.87 (d, J=8.6 Hz, 1H, Ar—H), 8.22 (d, J=8.8 Hz, 1H, Ar—H), 8.43 (s, 1H, Ar—H), 10.69 (s, 1H, OH).

### Biology

### Cells and Plasmids.

Cell lines used in this paper have been cultured in Dulbecco's Minimal Essential Medium (DMEM, Gibco, #11960-044), 10% heat-inactivated fetal bovine serum (A56-FBS), Penicillin/Streptomycin (Pen/Strep, Corning #20-002-C1), non-essential amino acids (NEAA, Gibco, #11140-035) and L-Glutamine (Gibco, #25030-024), unless specified differently. HEK293 cells were obtained from ATCC (ATCC CRL-1573). We used a subclone (A23) of HEK293 stably expressing a mouse WT, ΔCR, or EGFP-tagged PrP. Cells were passaged in T25 flasks or 100 mm Petri dishes in media containing 200 μg/ml of Hygromycin and split every 3-4 days. Cells have not been passaged more than 20 times from the original stock. Compounds used in the experiments were resuspended at 30 or 50 mM in DMSO, and diluted to make a 1000× stock solution, which was then used for serial dilutions. A 1 μl aliquot of each compound dilution point was then added to cells plated in 1 mL of media with no selection antibiotics. Cloning strategies used to generate cDNAs encoding for WT, ΔCR or EGFP-tagged PrP have been described previously^{20,31,32}. The EGFP-PrP construct contains a monomerized version of EGFP inserted after codon 34 of mouse PrP. The identity of all constructs was confirmed by sequencing the entire coding region. All constructs were cloned into the pcDNA3.1(+)/hygro expression plasmid (Invitrogen). All plasmids were transfected using Lipofectamine 2000 (Life Technologies), following manufacturer's instructions.

### Drug-Based Cell Assay (DBCA) and MTT Assay.

The DBCA was performed as described previously²⁴, with minor modifications. Briefly, HEK293 cells expressing ΔCR PrP were cultured at ˜60% confluence in 24-well plates on day 1. On day 2, cells were treated with 500 μg/mL of Zeocinfor. Medium (containing fresh Zeocin and/or compound or vehicle) was replaced every 24 hr. On day 5, cell medium was removed and cells were incubated with 1 mg/mL of 3-(4,5-dimethylthiazol-2-yl)-2,5-diphenyltetrazolium bromide (MTT, Sigma Aldrich, St. Louis, MO) in PBS for 30 min at 37° C. MTT was carefully removed, and cells were re-suspended in 500 μL of DMSO. Values for each well were obtained by measuring at 570 nm, using a plate spectrophotometer (Biotek).

### Electrophysiology.

Field Excitatory Post-Synaptic Potential (EPSP) mouse hippocampal slices of 11 weeks old C57BL/6 mice was measured with a Multi Electrode Array (MEA) system. Slices were recorded for a 30 minutes baseline, LTP was then induced with a tetanic stimulation (3 trains, 500 MHz each) and recorded for additional 30 minutes. Prion synaptotoxicity was induced by incubating the slices for 5 minutes during the baseline with a 4% w/v lysate of MoRK13 cells chronically infected with M1000 prion strain. In order to evaluate the potential rescuing activity of SM884, the molecule was continuously perfused during the whole recording. The percentage of LTP was calculated considering the average EPSP amplitude of the last 10 minutes of recording, over the average EPSP amplitude of the last five minutes before the tetanic stimulation.

### Immunofluorescence.

Cells expressing EGFP-PrP were plated on CellCarrier-384 Ultra microplates (Perkin Elmer) at a concentration of 12,000 cells/well and grown for approximately 24 h, to obtain a semi-confluent layer (60%). Vehicle (0.1% DMSO, volume equivalent) was used as a negative control. Cells were treated for 24 h and then fixed for 12 min at RT by adding methanol-free paraformaldehyde (Thermo Fisher Scientific) to a final concentration of 4%. Plates were then washed twice with PBS and counterstained with Hoechst 33342. The cell localization of EGFP-PrP was monitored using an Operetta High-Content Imaging System (Perkin Elmer). Imaging was performed in a widefield mode using a 20× High NA objective (0.75). Five fields were acquired in each well over two channels (380-445 Excitation-Emission for Hoechst and 475-525 for EGFP and Alexa 488). Image analysis was performed using the Harmony software version 4.1 (Perkin Elmer).

### Western Blotting.

Samples were diluted 1:1 in 2× Laemli sample buffer (2% SDS, 10% glycerol, 100 mM Tris-HCl pH 6.8, 0.002% bromophenol blue, 100 mM DTT), heated at 95° C. for 10 min, then analyzed by SDS-PAGE. Proteins were electrophoretically transferred to polyvinylidene fluoride (PVDF) membranes, which were then blocked for 20 min in 5% (w/v) non-fat dry milk in Tris-buffered saline containing 0.05% Tween-20. After incubation with appropriate primary and secondary antibodies, signals were revealed using enhanced chemiluminescence (Luminata, BioRad), and visualized by a Bio-Rad XRS Chemidoc image scanner (Bio-Rad).

### Preparation of Δβ Oligomers.

Synthetic Aβ (1-42) peptide (Cat. Number KP2107, Karebay Biochem., Rochester, NY) was dissolved in hexafluoro-2-propanol, incubated for 10 min in a bath sonicator at maximum power, centrifuged at 15.000×g for 1 min, aliquoted, dried, and stored at −80° C. Before use, the dried film was dissolved using DMSO and diluted to 100 μM in F12 Medium (Invitrogen, Waltham, MA). Oligomers were obtained by incubating the peptide for 16 h at 25° C. This preparation routinely produces oligomers that elute near the void volume of a Superdex 75 10/300 size exclusion column (GE Healthcare, Little Chalfont, UK), and that react with oligomer-specific antibody A11. Final Aβ oligomer concentrations were considered as monomer equivalents, since the size of the oligomers is heterogeneous.

### Cultured Hippocampal Neurons.

Primary neuronal cultures were derived from the hippocampi of 2-day-old postnatal mice, and cultured as described previously¹¹. Neurons were plated on 35-mm dishes (500,000 cells/dish) pre-coated with 25 μg/mL poly-D-lysine (Sigma P6407) in B27/Neurobasal-A medium supplemented with 0.5 mM glutamine, 100 units/mL penicillin, and 100 μg/mL streptomycin (all from Invitrogen). Experiments were performed 12 days after plating. Neurons were pre-treated for 20 min with each candidate compound or controls and then exposed for 20 mins or 3 hr to Aβ oligomers (3 μM). Triton-insoluble fractions (TIF) were analyzed by immunoblot with antibodies against phospho-SFK (Tyr 416) or Fyn. The phospho-SFK antibody detects pY416 in several SFKs, but previous studies showed that PrPC-dependent activation of kinases is specific for Fyn. Actin was used as loading control. Subcellular fractionation was performed as reported previously, with minor modifications. Neurons were homogenized using a Potter-Elvehjem homogenizer in 0.32 M ice-cold sucrose buffer (pH 7.4) containing 1 mM HEPES, 1 mM MgCl2, 10 mM NAF, 1 mM NaHCO₃, and 0.1 mM PMSF in the presence of protease inhibitors (Complete mini, Roche Applied Science, Penzberg, Germany) and phosphatase inhibitors (PhosSTOP, Roche Applied Science). Samples were centrifuged at 13.000×g for 15 min to obtain a crude membrane fraction. The pellet was re-suspended in buffer containing 150 mM KCl and 0.5% Triton X-100 and centrifuged at 100,000×g for 1 hr. The final pellet, referred to as the Triton-insoluble fraction, was re-homogenized in 20 mM HEPES supplemented with protease and phosphatase inhibitors and then stored at −80° C. or directly used in further experiments. Protein concentration in each sample was quantified using the Bradford assay (Bio-Rad), and proteins (5 μg) were then analyzed by Western blotting. Primary antibodies were as follow: anti-GluN2A and anti-GluN2B (both 1:2000; Invitrogen), anti-GluA1 and anti-GluA2 (both 1:1000; Millipore, Billerica, MA), anti-PSD-95 (post-synaptic density protein 95; 1:2000; Cayman Chemical, Ann Arbor, MI), and anti-actin (1:5000; Millipore). Western blots were analyzed by densitometry using Quantity One software (Bio-Rad). All experiments were repeated on at least 4 independent culture preparations (n≥4).

### Production of Recombinant PrP.

RecHuPrP23-231 was expressed by competent E. coli Rosetta (DE3) bacteria harboring pOPIN E expression vector containing a wild type human Prnp construct (N-KKRPKPGGWNTGGSRYPGQGSPGGNRYPPQGGGGWGQPHGGGWGQPHGGGWG QPHGGGWGQPHGGGWGQGGGTHSQWNKPSKPKTNMKHMAGAAAAGAVVGGL GGYMLGSAMSRPIIHFGSDYEDRYYRENMIHRYPNQVYYRPMDEYSNQNNFVHDC VNITIKQHTVTTTTKGENFTETDVKMMERVVEQMCITQYERESQAYYQRGSS-C; SEQ ID No. 1). Bacteria from a glycerolate maintained at −80° C. were grown in a 250 ml Erlenmeyer flask containing 50 ml of LB broth overnight. The culture was then transferred to two 2 L Erlenmeyer flasks containing each 500 ml of minimal medium supplemented with 3 g/L glucose, 1 g/L NH4Cl, 1M MgSO4, 0.1 M CaCl₂), 10 mg/mL thiamine and 10 mg/mL biotin. When the culture reached an OD600 of 0.9-1.2 AU, Isopropyl 3-D-1-thiogalactopyranoside (IPTG) was added to induce expression of PrP overnight under the same temperature and agitation conditions. Bacteria were then pelleted, lysed, inclusion bodies collected by centrifugation, and solubilized in 20 mM Tris-HCl, 0.5M NaCl, 6M Gnd/HCl, pH=8. Although the protein does not contain a His-tag, purification of the protein was performed with a histidine affinity column (HisTrap FF crude 5 ml, GE Healthcare Amersham) taking advantage of the natural His present in the octapeptide repeat region of PrP. After elution with buffer containing 20 mM Tris-HCl, 0.5M NaCl, 500 mM imidazole and 2 M guanidine-HCl, pH=8, the quality and purity of protein batches was assessed by BlueSafe (NZYTech, Lisbon) staining after electrophoresis in SDS-PAGE gels. The protein was folded to the PrPC conformation by dialysis against 20 mM sodium acetate buffer, pH=5. Aggregated material was removed by centrifugation. Correct folding was confirmed by CD and protein concentration, by measurement of absorbance at 280 nm. The protein was concentrated using Amicon centrifugal devices and the concentrated solution stored at −80° C. until used.

### Dynamic Mass Redistribution.

The EnSight Multimode Plate Reader (Perkin Elmer, Waltham, MA) was used to carry out DMR analyses. Immobilization of full-length (residues 23-230), human recombinant PrPC (15 μL/well of a 2.5 μM PrPC solution in 10 mM sodium acetate buffer, pH 5) on label-free microplates (EnSpire-LFB high sensitivity microplates, Perkin Elmer) was obtained by amine-coupling chemistry. The interaction between each molecule, diluted to different concentrations in assay buffer (10 mM P04, pH 7.5, 2.4 mM KCl, 138 mM NaCl, 0.05% Tween-20) and PrPC, was monitored after a 30 min incubation at room temperature. All the steps were executed by employing a Zephyr Compact Liquid Handling Workstation (Perkin Elmer). The Kaleido software (Perkin Elmer) was used to acquire and process the data.

### Statistical Analyses of Biological Data.

All the data were collected and analyzed blindly by two different operators. Statistical analyses, performed with the Prism software version 7.0 (GraphPad), included all the data points obtained, with the exception of experiments in which negative and/or positive controls did not give the expected outcome, which were discarded. No test for outliers was employed. The Kolmogorov-Smirnov normality test was applied (when possible, n≥5). Results were expressed as the mean±standard errors, unless specified. In some case, the dose-response experiments were fitted with a 4-parameter logistic (4PL) non-linear regression model, and fitting was estimated by calculating the R². All the data were analyzed with the one-way ANOVA test, including an assessment of the normality of data, and corrected by the Dunnet post-hoc test. Probability (p) values <0.05 were considered as significant (*<0.05, **<0.01, ***<0.001).

### In Vitro Bone Marrow-Derived Dendritic Cells

Bone marrow cells were isolated from C57BL/6 mice as previously describe (DOI: 10.1073/pnas.1619863114). BM was harvested from femur, tibia and pelvis using mortar and pestle in 1×PBS supplemented with 0.5% BSA and 2 mM EDTA (MACS buffer), passed through a 70 μm cell strainer and centrifuged at 1400 r.p.m for 5 minutes. Red blood cells were lysed with ACK lysis buffer (Ammonium Chloride 0.15 M, Potassium Carbonate 10 mM) and debris were removed by a gradient centrifugation using Histopaquel 119 (#11191, Sigma-Aldrich) prior to culture. Cells were resuspend at 2×106 cells/ml in Iscove's Modified Dulbecco's Media (IMDM, #12440053, Thermo Fisher) supplemented with 0.1 Non-essential Amminoacids (#11140-035 Thermo Fisher), 1 mM Sodium Pyruvate (#11360-070, Thermo Fisher), 5 mM glutamine (#25030-024, Thermo Fisher), 50 μM 2-Mercaptoethanol (#31350-010, Thermo Fisher), 100 U/ml penicillin, 100 g/ml streptomycin (#15140-122, Thermo Fisher) and 10% FBS (#10270-106, Thermo Fisher) (complete IMDM) containing 5% murine Flt3-L and were seed 5 ml/well in 6-plate tissue culture plates at 37° C. for 8-10 days. For all culture experiments, loosely adherent and suspension cells were harvested by gentle pipetting at the indicated time point.

cDC1 and cDC2 were sorted into complete IMDM were sorted by FACSAria Fusion as pDC B220+Bst2+, cDC1 B220-CD11c+MHC-II+CD24+CD172α−, cDC2 as B220-CD11c+MHCII+CD24-CD172α+. Sort purity of >95% was confirmed by post-sort analysis before cells were used for further experiments.

### Induction of EAE

All mice used were 12 weeks animals on the C57BL/6 background. EAE was induced with 200 μg of myelin oligodendrocyte glycoprotein fragment MEVGWYRSPFSRVVHLYRNGK (SEQ ID No. 2; MOG35-55 peptide; #crb1000205n Cambridge Research Biochemicals) mixed with incomplete Freund's Adjuvant (#263910, BD) containing 4 mg/ml Mycobacterium tuberculosis TB H37 Ra (#231141 BD), at a ratio of 1:1 (v/v). Mice received 2 subcutaneous injections of 100 μl each of the MOG/CFA mix. Mice then received a single intraperitoneal injection of pertussis toxin (#180, List Biological Laboratories) at a concentration of 1 ng/μL in 200 μL of PBS. Mice received a second injection of pertussis toxin at the same concentration two days after the initial EAE induction. Mice were orally treated with different doses of SM231 dissolved in 1×PBS on alternating days starting at day 10 post-EAE induction. Mice were monitored and scored daily thereafter. EAE clinical scores were defined as follows: 0—no signs, 1—fully limp tail, 2—hindlimb weakness, 3—hindlimb paralysis, 4—forelimb paralysis, 5—moribund, as described previously (Mayo et al., 2014; Rothhammer et al., 2016). Sex differences were not analyzed but only a single sex was used within any set of EAE experiments. Mice were randomly assigned to treatment groups.

### Results

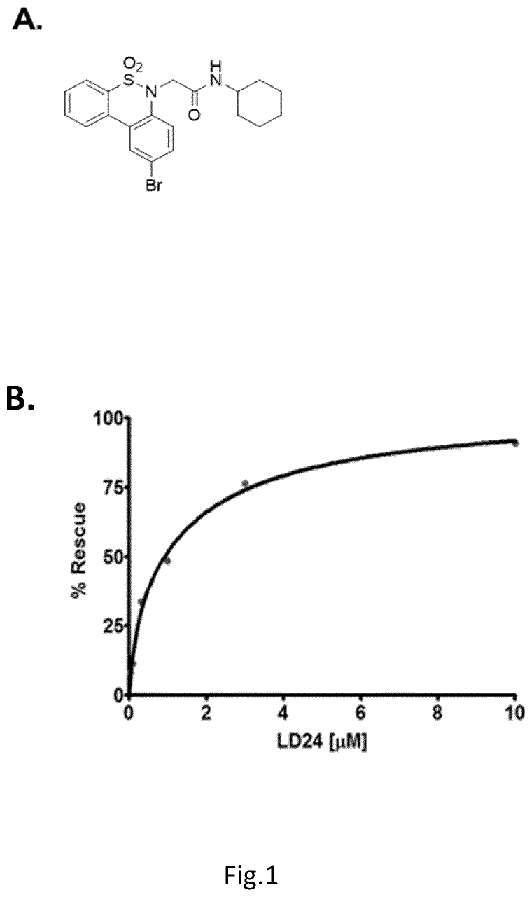

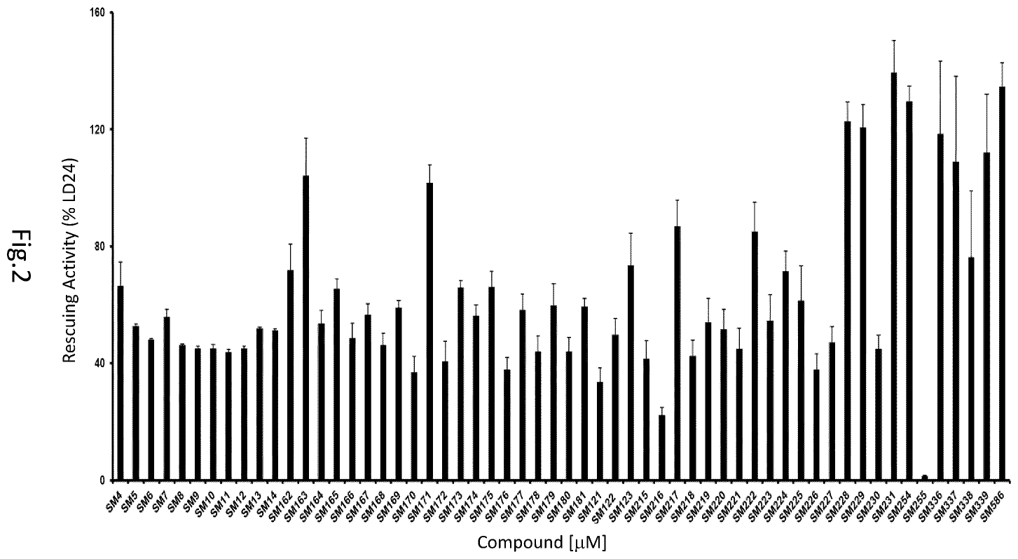

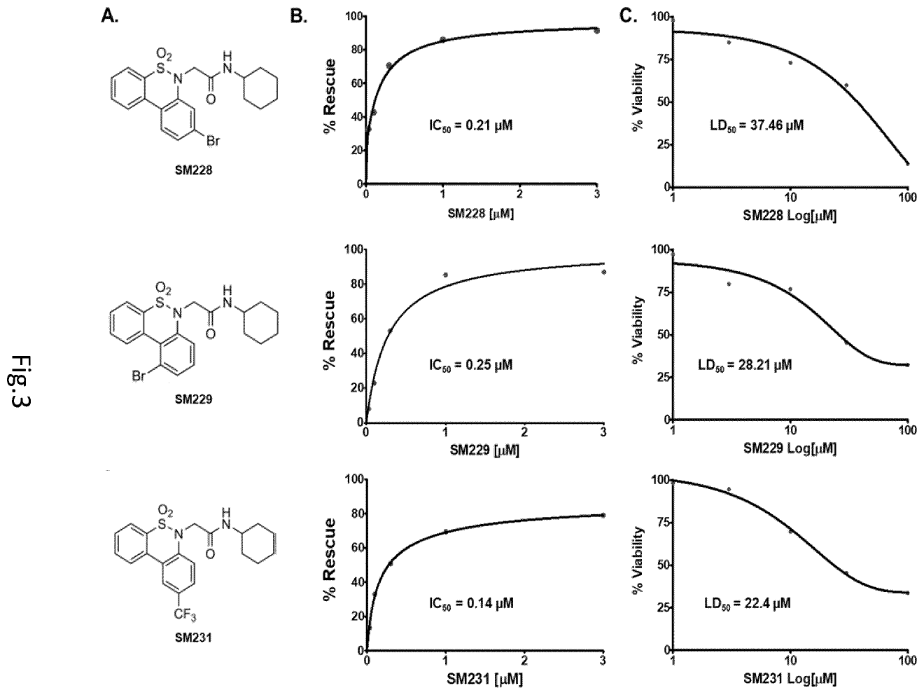

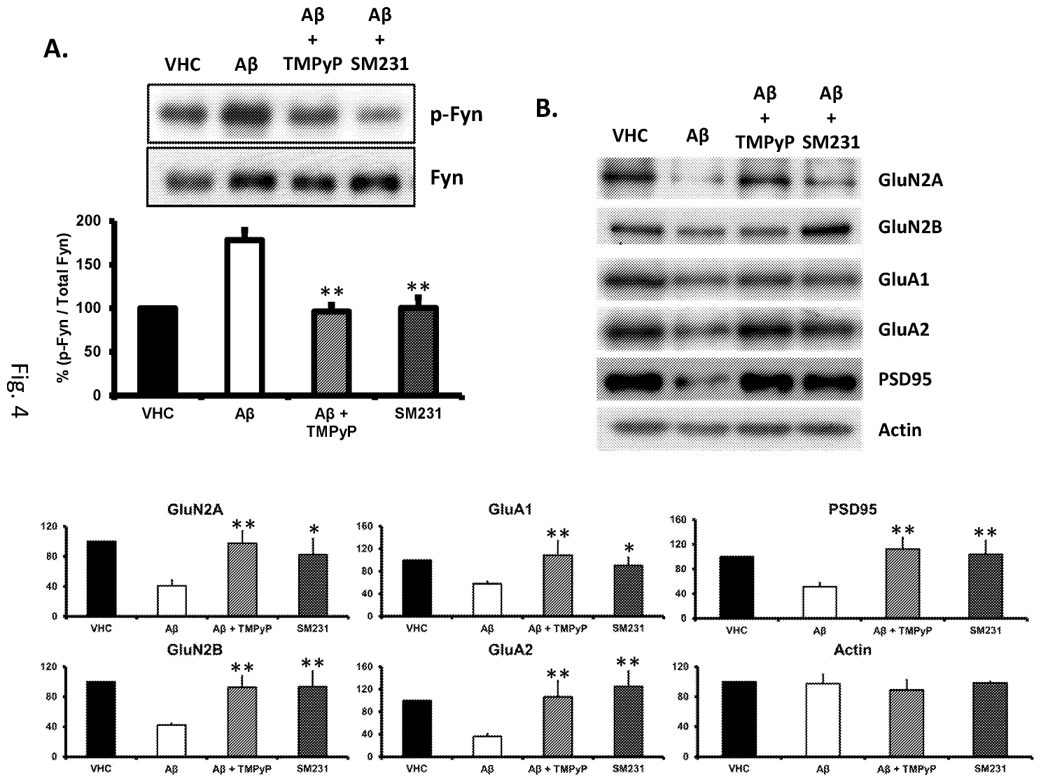

Identification, characterization and optimization of SM3. Mutations in the central region of PrPC, including artificial deletions or disease-associated point mutations, induce a toxic ion channel activity that can be detected in transfected cells by patch-clamping techniques^{23,24}. Cells expressing PrP mutants are also hypersensitive to several cationic drugs commonly used for selection of transfected cell lines, including aminoglycosides and phleomycin analogues²⁰. The latter effect was used to establish a novel cellular assay for studying mutant PrPC-related toxicity, called the “drug-based cell assay”, or DBCA²⁵. Importantly, co-expression of wild type (WT) PrPC suppresses both channel activity and citoxicity, likely indicating that mutant PrP forms aberrantly activate a signaling pathway normally regulated by PrPC. Thus, the DBCA represents a unique tool to identify compounds capable of modulating PrPC activity. We have developed an optimized and scaled-up format of the DBCA in 384-well plates, which was later employed to screen tens of thousands of small molecules^{21,22}. Several compounds were found to suppress the toxicity of mutant PrP, with no detectable toxicity in WT cells. We focused efforts on one of these compounds (named SM3 [dibenzo [3,4][c,e]thiazine 5,5-dioxide], shown in FIG. 1A). SM3 possesses a drug-like chemical scaffold suitable for optimization and structure-activity relationship (SAR) experiments. Tens of derivatives (FIG. 1C) were designed and synthesized, and their biological activity tested by DBCA. Three chemical regions of the compound were explored, with the dual objective of improving potency and acquiring SAR information. Taking as reference the biological activity of the parent compound SM3 (FIG. 1i), we evaluated the activity of the different derivatives (FIG. 2). We made several important observations about SM3. Chemical modifications made at the spacer region were not fruitful. Conversely, substitutions at the C ring improved potency, with the 9-CF3 derivatives being the most potent. Branched substituents on the cyclohexyl group were not tolerated, whereas substituted phenyl rings generated analogues with potency comparable to the reference compound. Collectively, these results provided important SAR information about SM3, and directly suggested chemistry schemes to engineer additional derivatives and functionalized analogues. Moreover, we identified a potent derivative, called SM231, which showed activity by the DBCA in the sub-micromolar range (FIG. 3).

TABLE 3
Protective effect on HEK293 cells: the value is expressed as Rescue percent (% R_{MAX})
produced by target compounds with respect to hit molecule SM3; IC₅₀ and LD₅₀ of target
compounds derived from DBCA.

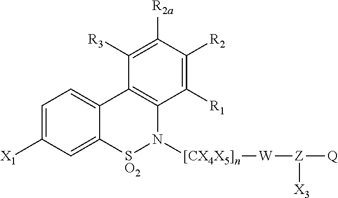

| LAC code | X₁ | 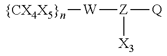 | R₁ | R₂ | R₂ₐ | R₃ | % R_{MAX}¹ | IC₅₀² | LD₅₀³ |
|---|---|---|---|---|---|---|---|---|---|
| SM3 | H | 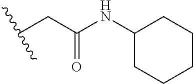 [SMILES: *CC(=O)NC1CCCCC1] | H | H | Br | H | 100 | 1.1 | >100 |
| SM7 | H | 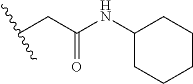 [SMILES: *CC(=O)NC1CCCCC1] | H | H | H | H | 55.93 | — | — |
| SM8 | H | 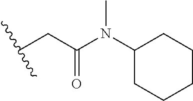 [SMILES: *CC(=O)N(C)C1CCCCC1] | H | H | Br | H | 46.2 | — | — |
| SM9 | H | 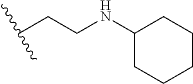 [SMILES: *CCNC1CCCCC1] | H | H | Br | H | 45.18 | — | — |
| SM10 | H | 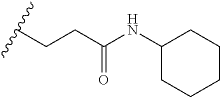 [SMILES: *CCC(=O)NC1CCCCC1] | H | H | Br | H | 45.06 | — | — |
| SM11 | H | 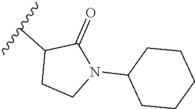 [SMILES: *C1CCN(C2CCCCC2)C1=O] | H | H | Br | H | 43.87 | — | — |
| SM225 | H | 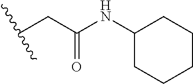 [SMILES: *CC(=O)NC1CCCCC1] | H | H | OMe | H | 61.53 | — | — |
| SM226 | H | 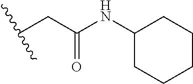 [SMILES: *CC(=O)NC1CCCCC1] | H | H | OH | H | 37.89 | — | — |
| SM227 | H | 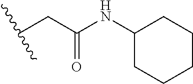 [SMILES: *CC(=O)NC1CCCCC1] | Br | H | H | H | 41.15 | — | — |
| SM228 | H | 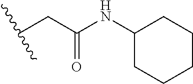 [SMILES: *CC(=O)NC1CCCCC1] | H | Br | H | H | 122.76 | 0.21 | 37.46 |
| SM229 | H | 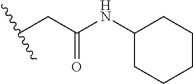 [SMILES: *CC(=O)NC1CCCCC1] | H | H | H | Br | 120.78 | 0.25 | 28.21 |
| SM230 | H | 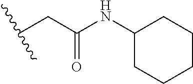 [SMILES: *CC(=O)NC1CCCCC1] | H | H | 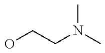 | H | 44.94 | — | — |
| SM231 | H | 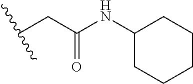 [SMILES: *CC(=O)NC1CCCCC1] | H | H | CF₃ | H | 139.52 | 0.14 | 22.4 |
| SM338 | H | 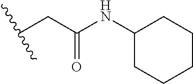 [SMILES: *CC(=O)NC1CCCCC1] | H | CF₃ | H | H | 87.62 | — | — |
| SM339 | H | 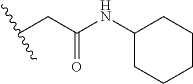 [SMILES: *CC(=O)NC1CCCCC1] | H | H | H | CF₃ | 112.24 | 0.34 | — |
| SM254 | H | 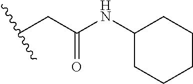 [SMILES: *CC(=O)NC1CCCCC1] | H | H | Cl | H | 126.52 | 0.3 | — |
| SM585 | H | 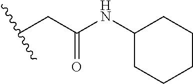 [SMILES: *CC(=O)NC1CCCCC1] | H | H | H | Cl | 71.71 | — | — |
| SM586 | H | 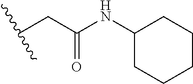 [SMILES: *CC(=O)NC1CCCCC1] | H | Cl | H | H | 181.02 | 0.35 | — |
| SM340 | H | 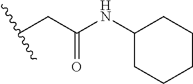 [SMILES: *CC(=O)NC1CCCCC1] | H | H | SMe | H | 22.96 | — | — |
| SM587 | H | 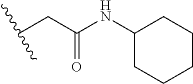 [SMILES: *CC(=O)NC1CCCCC1] | H | Cl | H | Cl | 167.88 | 0.6 | — |
| SM336 | H | 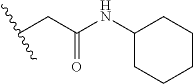 [SMILES: *CC(=O)NC1CCCCC1] | H | Cl | F | H | 118.6 | 0.1 | — |
| SM337 | H | 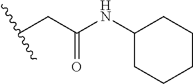 [SMILES: *CC(=O)NC1CCCCC1] | H | H | F | Cl | 109.06 | 0.97 | — |
| SM588 | H | 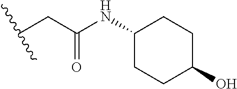 [SMILES: *CC(=O)N[C@H]1CC[C@H](O)CC1] | H | H | CF₃ | H | 10.56 | — | — |
| SM589 | H | 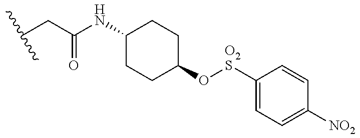 [SMILES: *CC(=O)N[C@H]1CC[C@H](OS(=O)(=O)c2ccc([N+](=O)[O-])cc2)CC1] | H | H | CF₃ | H | 116.12 | 1.05 | — |
| SM656 | H | 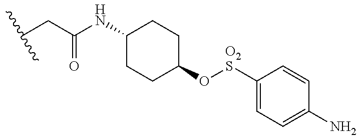 [SMILES: *CC(=O)N[C@H]1CC[C@H](OS(=O)(=O)c2ccc(N)cc2)CC1] | H | H | CF₃ | H | NS | NS | NS |
| SM655 | H | 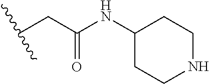 [SMILES: *CC(=O)NC1CCNCC1] | H | H | CF₃ | H | NS | NS | NS |
| SM880 | H | 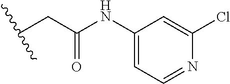 [SMILES: *CC(=O)Nc1ccnc(Cl)c1] | H | H | CF₃ | H | 78.26 | — | — |
| SM881 | F | 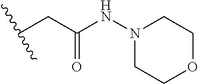 [SMILES: *CC(=O)NN1CCOCC1] | H | H | CF₃ | H | 150.19 | 0.032 | >100 |
| SM882 | F | 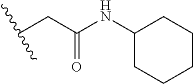 [SMILES: *CC(=O)NC1CCCCC1] | H | H | CF₃ | H | 138.54 | 0.044 | >100 |
| SM883 | OEt | 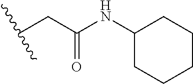 [SMILES: *CC(=O)NC1CCCCC1] | H | H | CF₃ | H | 165.5 | 0.014 | >100 |
| SM884 | F | 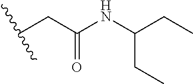 [SMILES: *CC(=O)NC(CC)CC] | H | H | CF₃ | H | 210.54 | 0.018 | >100 |
| SM885 | F | 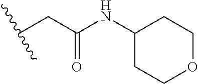 [SMILES: *CC(=O)NC1CCOCC1] | H | H | CF₃ | H | 123.14 | 0.053 | >100 |
| SM879 | | 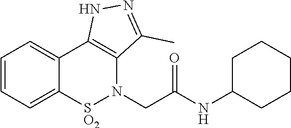 [SMILES: Cc1n[nH]c2c1N(CC(=O)NC1CCCCC1)S(=O)(=O)c1ccccc1-2] | | | | | 22.54 | — | — |
| SM4 | H | 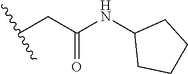 [SMILES: *CC(=O)NC1CCCC1] | H | H | Br | H | 66.56 | — | — |
| SM5 | H | 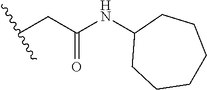 [SMILES: *CC(=O)NC1CCCCCC1] | H | H | Br | H | 52.82 | — | — |
| SM6 | H | 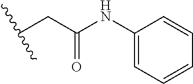 [SMILES: *CC(=O)Nc1ccccc1] | H | H | Br | H | 48.1 | — | — |
| SM162 | H | 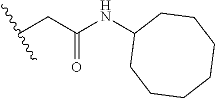 [SMILES: *CC(=O)NC1CCCCCCC1] | H | H | Br | H | 71.81 | — | — |
| SM163 | H | 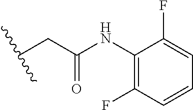 [SMILES: *CC(=O)Nc1c(F)cccc1F] | H | H | Br | H | 104.28 | — | — |
| SM164 | H | 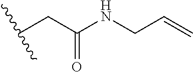 [SMILES: *CC(=O)NCC=C] | H | H | Br | H | 53.67 | — | — |
| SM165 | H | 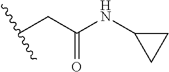 [SMILES: *CC(=O)NC1CC1] | H | H | Br | H | 65.56 | — | — |
| SM166 | H | 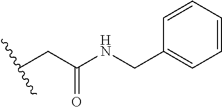 [SMILES: *CC(=O)NCc1ccccc1] | H | H | Br | H | 48.61 | — | — |
| SM167 | H | 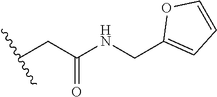 [SMILES: *CC(=O)NCc1ccco1] | H | H | Br | H | 56.63 | — | — |
| SM168 | H | 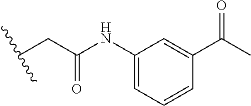 [SMILES: *CC(=O)Nc1cccc(C(C)=O)c1] | H | H | Br | H | 46.21 | — | — |
| SM169 | H | 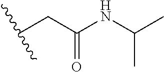 [SMILES: *CC(=O)NC(C)C] | H | H | Br | H | 59.15 | — | — |
| SM170 | H | 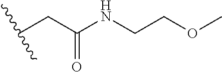 [SMILES: *CC(=O)NCCOC] | H | H | Br | H | 36.94 | — | — |
| SM171 | H | 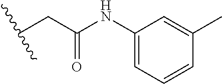 [SMILES: *CC(=O)Nc1cccc(C)c1] | H | H | Br | H | 101.77 | — | — |
| SM172 | H | 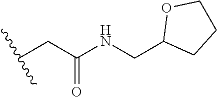 [SMILES: *CC(=O)NCC1CCCO1] | H | H | Br | H | 40.61 | — | — |
| SM173 | H | 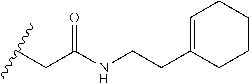 [SMILES: *CC(=O)NCCC1=CCCCC1] | H | H | Br | H | 66.03 | — | — |
| SM174 | H | 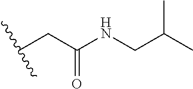 [SMILES: *CC(=O)NCC(C)C] | H | H | Br | H | 56.28 | — | — |
| SM175 | H | 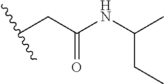 [SMILES: *CC(=O)NC(C)CC] | H | H | Br | H | 66.2 | — | — |
| SM176 | H | 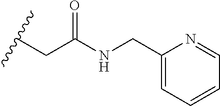 [SMILES: *CC(=O)NCc1ccccn1] | H | H | Br | H | 37.85 | — | — |
| SM177 | H | 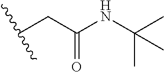 [SMILES: *CC(=O)NC(C)(C)C] | H | H | Br | H | 58.41 | — | — |
| SM178 | H | 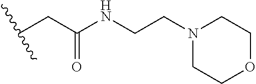 [SMILES: *CC(=O)NCCN1CCOCC1] | H | H | Br | H | 44.01 | — | — |
| SM179 | H | 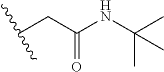 [SMILES: *CC(=O)NC(C)(C)C] | H | H | Cl | H | 59.78 | — | — |
| SM180 | H | 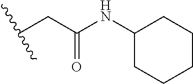 [SMILES: *CC(=O)NC1CCCCC1] | H | H | i-Pr | H | 44.05 | — | — |
| SM181 | H | 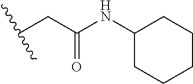 [SMILES: *CC(=O)NC1CCCCC1] | H | H | Me | H | 59.35 | — | — |
| SM219 | H | 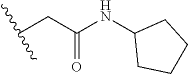 [SMILES: *CC(=O)NC1CCCC1] | H | H | F | H | 53.97 | — | — |
| SM220 | H | 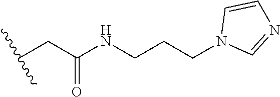 [SMILES: *CC(=O)NCCCn1ccnc1] | H | H | Br | H | 51.7 | — | — |
| SM221 | H | 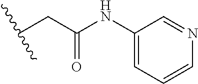 [SMILES: *CC(=O)Nc1cccnc1] | H | H | Br | H | 44.87 | — | — |
| SM222 | H | 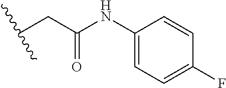 [SMILES: *CC(=O)Nc1ccc(F)cc1] | H | H | Br | H | 85.07 | — | — |
| SM223 | H | 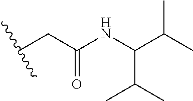 [SMILES: *CC(=O)NC(C(C)C)C(C)C] | H | H | Br | H | 54.69 | — | — |
| SM224 | H | 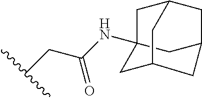 [SMILES: *CC(=O)NC12CC3CC(CC(C3)C1)C2] | H | H | F | H | 71.5 | — | — |
| SM886 | H | 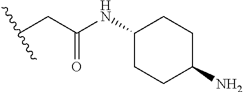 [SMILES: *CC(=O)N[C@H]1CC[C@H](N)CC1] | H | H | CF₃ | H | NT | — | — |
| SM887 | H | 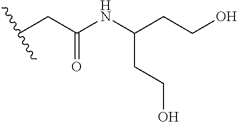 [SMILES: *CC(=O)NC(CCO)CCO] | H | H | CF₃ | H | ~50 | — | — |
| SM888 | F | 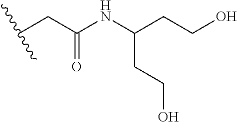 [SMILES: *CC(=O)NC(CCO)CCO] | H | H | CF₃ | H | ~50 | — | — |
| SM889 | F | 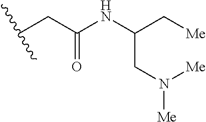 [SMILES: *CC(=O)NC(CC)CN(C)C] | H | H | CF₃ | H | ~50 | — | |
| SM890 | OH | 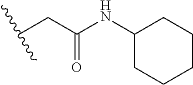 [SMILES: *CC(=O)NC1CCCCC1] | H | H | CF₃ | H | NT | — | — |
| SM891 | OH | 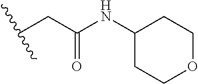 [SMILES: *CC(=O)NC1CCOCC1] | H | H | CF₃ | H | NT | — | — |
| SM892 | OH | 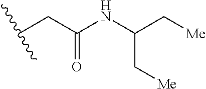 [SMILES: *CC(=O)NC(CC)CC] | H | H | CF₃ | H | NT | — | |

Notes:
1. R_{MAX} indicates the maximum rescuing effect at the concentration of 1 μM, expressed as percentage of the effect of SM3(LD24) at the same concentration.
2. Rescuing dose at 50%, expressed in μM. This is typically obtained by testing a 8-point dose-response curve, performed only for compounds showing an improved activity (>110%) as compared to SM3(LD24).
3. Lethal dose at 50%, expressed in μM. This is typically obtained by testing a 8-point dose-response curve, performed only for selected compounds.
NS = not soluble, data obtained from these compounds are not reliable due to solubility issues
NT = not tested

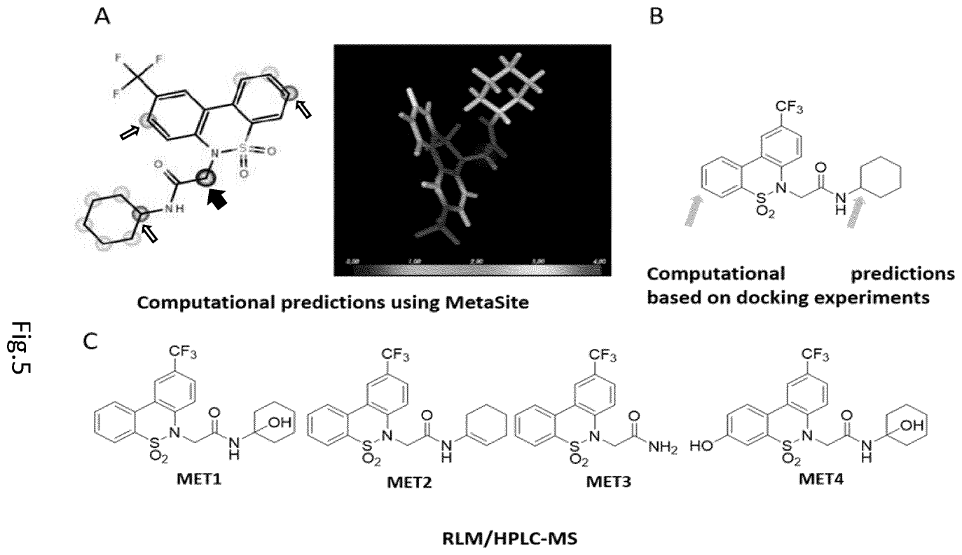

SM231 inhibits the synaptotoxic effects of Aβ oligomers. Recent studies identified a role for PrPC into the toxicity of various misfolded oligomers of diseases-associated proteins, such as the amyloid B, whose accumulation underline the cognitive decline occurring in Alzheimer's disease^{2,4}. The interaction between PrPC and Aβ oligomers unleashes a rapid, toxic signaling pathway involving the metabotropic glutamate receptor 5 (mGluR5), activation of the tyrosine kinase Fyn, and phosphorylation of the NR2B subunit of NMDA receptor, ultimately producing dysregulation of receptor function, excitotoxicity and dendritic spine retraction¹². In order to evaluate the effect of SM231 on Aβ-induced activation of Fyn, we exposed primary hippocampal neurons to different concentrations of Aβ oligomers for short times (10, 20 or 60 minutes). We confirmed that the oligomers induce a quick phosphorylation of the Fyn kinase (results at the 20 min time point are shown in FIG. 4). Consistent with previous observations²⁶, this effect was prevented by treatment with a PrPC-directed compound [called Fe(III)-TMPyP]³⁵. Interestingly, co-incubation with SM231 completely abrogated Aβ effects, restoring Fyn phosphorylation to normal levels (FIG. 4A). Next, we directly tested the ability of SM231 to block Aβ oligomer-dependent synaptotoxicity. Primary hippocampal neurons were incubated for 3 hours with Aβ oligomers (3 μM). Consistent with previous reports¹¹, we observed a decrease of several post-synaptic markers (subunits of NMDA receptor, GluN2A and GluN2B, and AMPA, GluA1 and GluA2, and the post-synaptic density protein 95, PSD-95), as evaluated by western blotting of the triton-insoluble fractions. These effects were rescued by treatment with anti-PrPC molecule Fe(III)-TMPyP. Importantly, co-incubation with SM231 for 20 minutes significantly rescued the levels of all the post-synaptic markers. The level of a control protein (actin) was not affected by either Aβ oligomers or SM231. These data demonstrate that SM231 inhibits the ability of Aβ oligomers to subvert the function of PrPC and activate a neurotoxic signaling pathway.

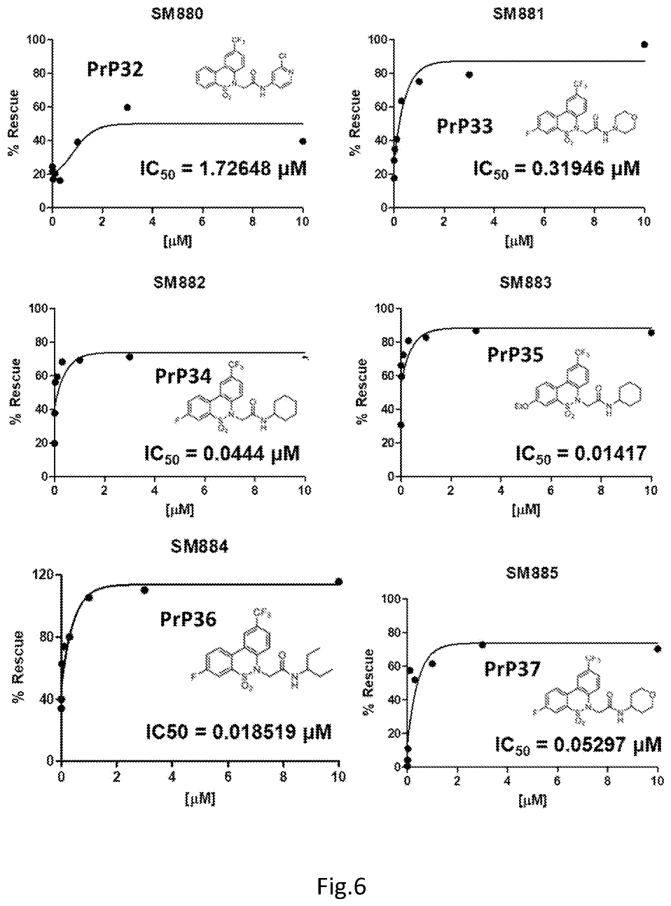

Chemical optimization of SM231 to more metabolically stable derivatives. Within the present invention it was carried-out a further chemical optimization cycle functionalizing positions predicted to positively improve the metabolic stability (FIG. 5). In particular, the C-3 position of the dibenzothiazine nucleus was functionalized by a F and an EtO (SM882 and SM883 derivatives, respectively) while in other three molecules the cyclohexyl was replaced by a more stable and hydrophilic groups (morpholine and tetrahydropyrane) or opened to give a branched chain (SM881, SM884, and SM885). Interestingly, when assayed on DBCA, compound SM884 resulted more potent than SM231 (FIG. 6), making these two molecules as promising lead compounds for further development.

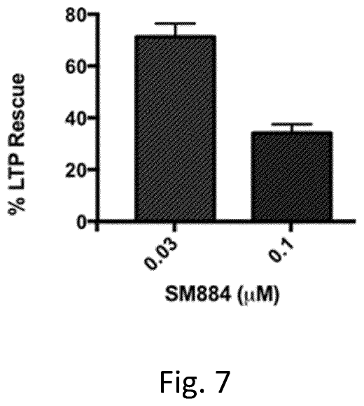

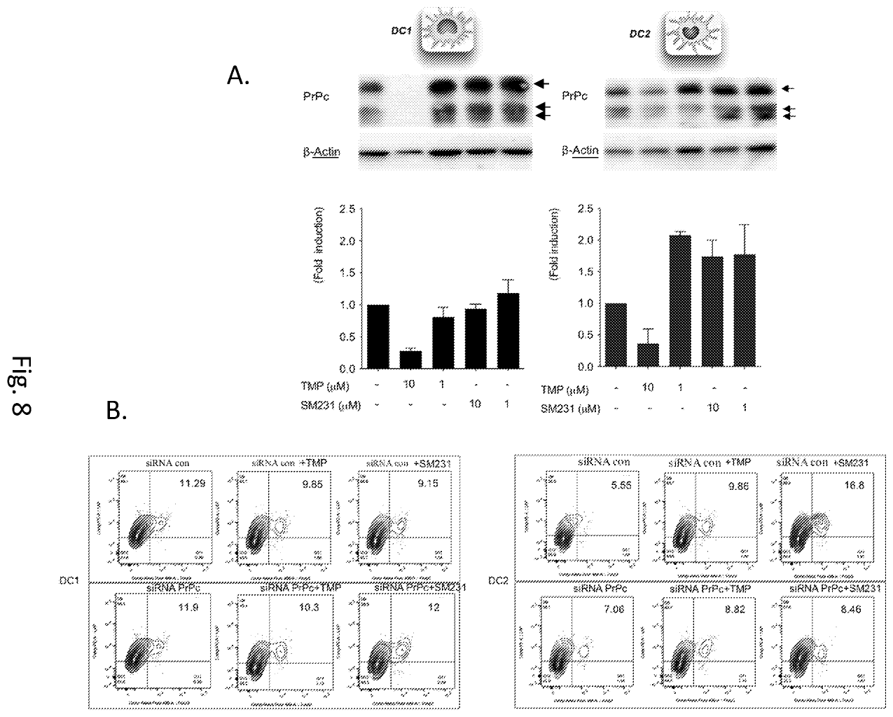

SM884 rescues the synaptotoxic effects of prions in mouse brain slices. To test whether SM884 is able to inhibit prion-induced toxicity in a disease-relevant context, we turned to a recently developed ex vivo toxicity model^{27,28}. This assay is based on mouse brain slices acutely exposed to either brain homogenates of terminally ill mice infected with lysates of cell lines chronically infected with the mouse-adapted M1000 human prion strain. We found that SM884 administration at a concentration of 0.1-0.03 μM induces a significant (34% and 71%, respectively) rescue of long-term potentiation (LTP; FIG. 7). The higher potency detected at the lowest dose likely reflected an observed aggregation propensity of the molecule in the experimental conditions. These results were also fully consistent with the estimated half-maximal rescuing dose of the compound in cells (0.018 μM), as evaluated by DBCA, and clearly showed that SM884 is capable of suppressing the synaptic impairment induced by prions in the low nanomolar concentration range.

Mouse DC1 and DC2 subsets express PrPC, and DC2 treated with SM231 promotes Treg cells expansion in DC-T cell co-cultures. Bone marrow derived dendritic cells were analyzed for expression of PrPC after stimulation with two different concentrations of SM231 or Fe(III)-TMPyP or vehicle. For this analysis, PrPC expression in each DC subsets was determined by western blot using specific anti-PrPC antibody. The authors of the present invention found that DC1 and DC2 expressed a baseline level of PrPC that slightly increases upon SM231 treatment, especially in DC2 (FIG. 8A). To assess the inhibitory function of DC1 or DC2 cells after treatment with SM231 or Fe(III)-TMPyP we performed in vitro co-cultures of DCs with naïve CD4+ T cells. It was found that the priming ability of conventional DC2 was significantly affected by DC2 treatment with SM231. Specifically, these cells were able to favor the expansion of T cells expressing Treg cell markers FoxP3 and LAP and this effect required PrPC expression in DCs, since it was prevented in DC2 cells that were transfected by a specific PrPC siRNA but not by a control siRNA (FIG. 8B). Overall, these data suggest that PrPC stimulation may confer tolerogenic function to DC2, suppressing the default immunogenic program of this subset.

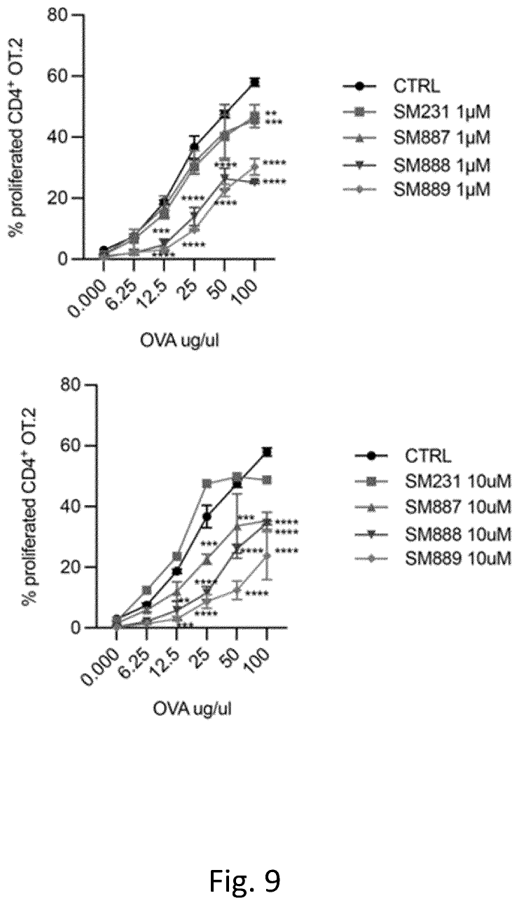

Compounds SM888 and SM889, like SM231, promote tolerogenic activity in cDC2. cDC2 cells have been reported to trans-present IL-6 indispensable for priming myelin peptide specific encephalitogenic pathogenic TH17 in a model of EAE. To assess whether additional derivates (i.e SM887, SM888, SM889) were able to induce regulatory functions in DCs subsets we performed in vitro co-cultures of cDC2 cells with naïve ovalbumin (OVA)-specific transgenic CD4+ T cells in the presence of different concentrations of OVA. T cell proliferation was analyzed. We found that priming of cDC2 was significantly affected by cDC treatment with SM derivatives and more significantly by SM888 and SM889. Specifically, these cells were able to suppress antigen-specific CD4+ T cell proliferation and this effect was more pronounced when the molecules were used at the concentration of 10 uM (FIG. 9).

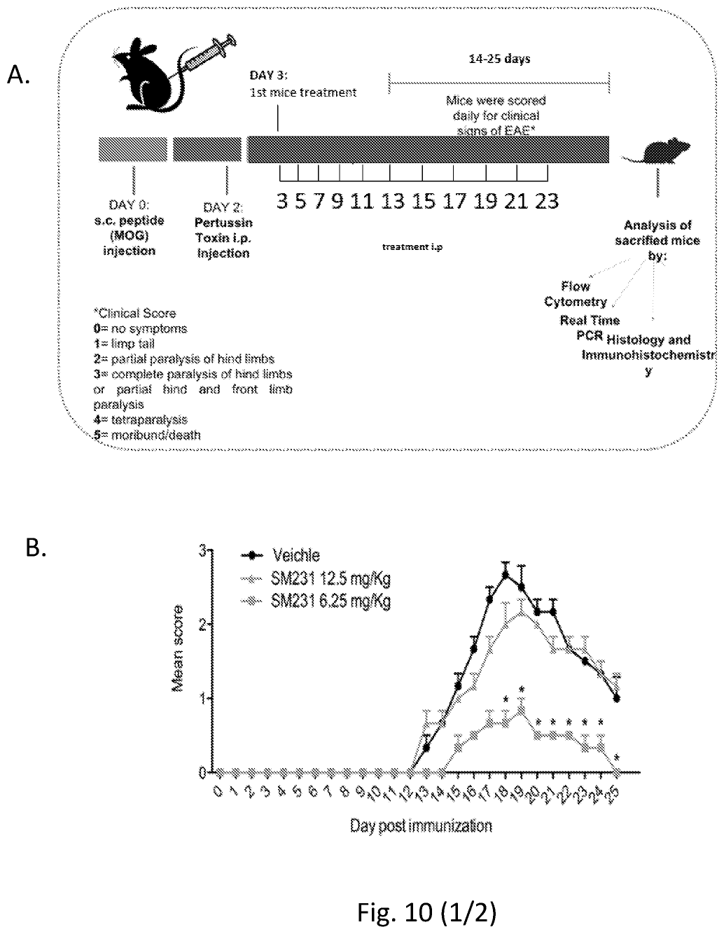

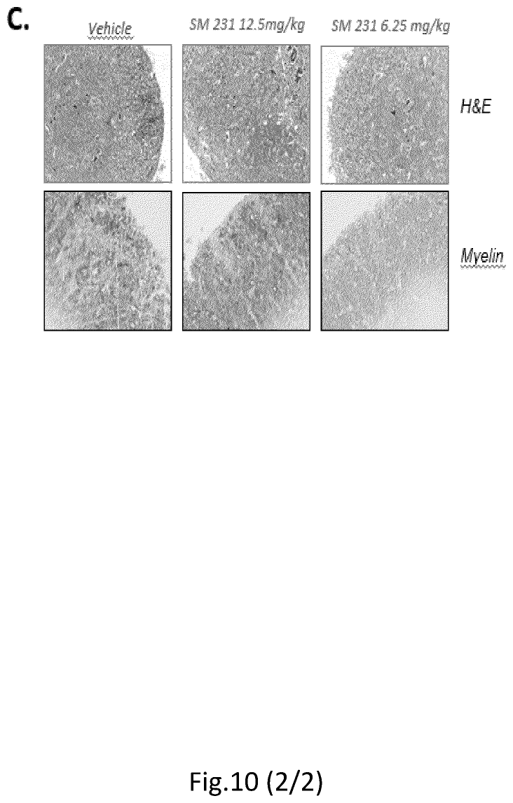

Administration SM231 ameliorates EAE and suppresses inflammatory cytokines in vivo. The authors of the invention investigated whether PrPC modulators could have a protective role in this experimental model. Groups of WT female C57BL/6 mice were immunized with the MOG35-55 peptide and injected intraperitoneally (i.p.) with Fe(III)-TMPyP or SM231 at two doses every other day from day 3 until day 24 after vaccination. Control mice received vehicle alone. EAE clinical scores were recorded daily over this timeframe (FIG. 10A). We found that SM231 (FIG. 10B) administration resulted in a reduced disease as compared to control mice (P<0.05). At d 25 post-vaccination, white matter demyelination and inflammatory infiltrates were reduced by SM231 compared to vehicle treated control (FIG. 10C). Moreover, SM231 in vivo treatment resulted in a reduced secretion of inflammatory cytokines such as IL-17A and GM-CSF by CD4+ T cells purified from cervical lymph nodes and re-stimulated with MOG in vitro. These data support the therapeutic and also physiologic value of PrPC activation in the control of neuroinflammation. Moreover, these data suggest that molecules such as SM231 may exert these effects by regulating potential inflammatory antigen presenting cells also in vivo. Importantly, these data were similar to those obtained with other derivatives of SM231.

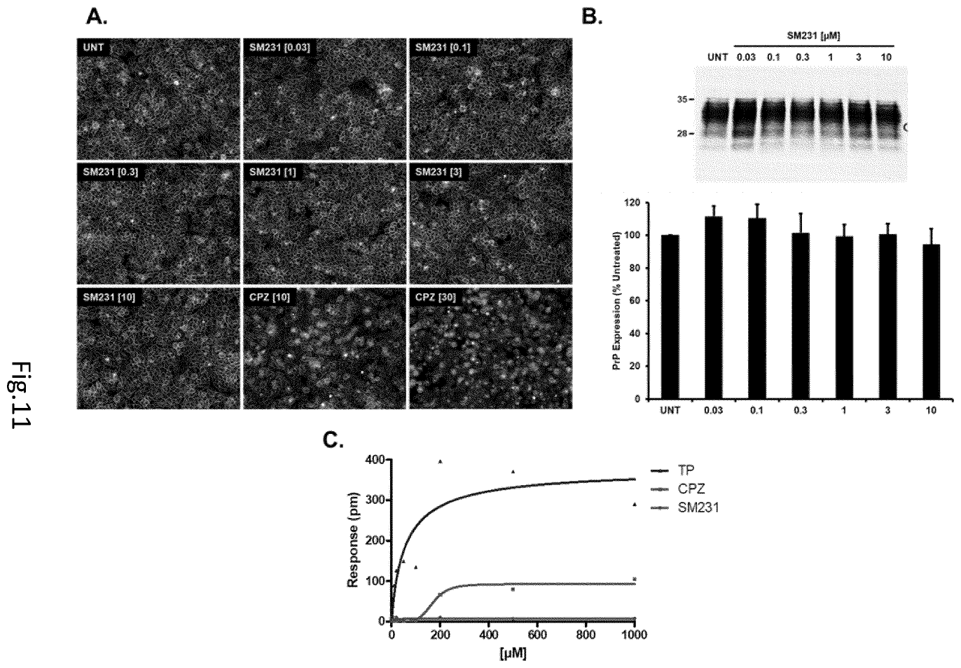

SM231 does not act by directly targeting PrPC. In light of the promising ability of SM compounds to modulate the activity of PrPC in several experimental contexts, the hypothesis that these molecules act by directly targeting the protein was tested. First, it was hypothesized that the compound may promote the re-localization of PrPC from the cell surface, a mode of action recently observed for an anti-prion phenothiazine derivative (chlorpromazine, CPZ)^{29,30}. HEK293 cells stably expressing an EGFP-tagged version of PrPC were treated with different concentration of SM231, CPZ or vehicle control, and PrPC localization at the cell surface was monitored by imaging techniques (FIG. 11A). Results showed that CPZ induced a dose-dependent relocalization of EGPF-PrPC from the cell surface to intracellular compartments, in line with previous data³⁰. Conversely, no changes were detected for SM231, suggesting that this compound does not exert its effects by inducing the relocalization of PrPC from the cell surface. Next, it was tested whether SM231 could alter PrPC expression. HEK293 cells stably expressing wild-type PrPC were treated with SM231 at different concentrations. Total PrPC levels were then evaluated in whole-cell lysates by western blotting (FIG. 11B). Once again, no difference in PrPC expression was found upon treatment with SM231. Finally, the direct binding of SM231 to recombinant PrPC was tested by dynamic mass redistribution (DMR), a biophysical technique previously employed to detect the interaction of small molecules to PrPC²⁶. Fe(III)-TMPyP and chlorpromazine (CPZ), two compounds previously reported to show respectively high and low affinity for PrPC^{19,30} were used as control. However, no interaction was observed between SM231 and recombinant PrPC, even at the highest concentration (1 mM, FIG. 11C). Collectively, these results indicate that SM231 does not act by directly binding PrPC, or by altering its expression or localization.

An FXR-inhibitor suppresses mutant PrP cytotoxicity. The two FXR agonists, WAY-362450 and Fexaramine, whose structure is reported below, were tested using the DBCA assay.

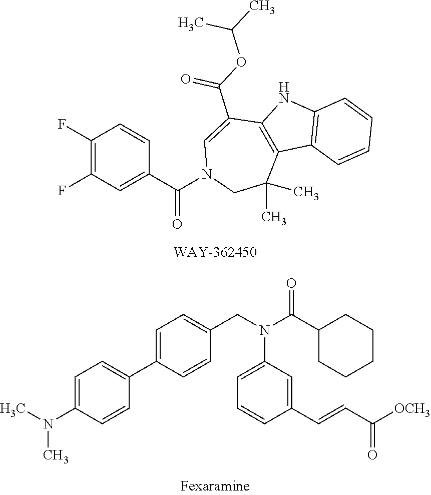 [SMILES: CC(C)OC(=O)C1=CN(C(=O)c2ccc(F)c(F)c2)CC(C)(C)c2c1[nH]c1ccccc21 ; COC(=O)/C=C/c1cccc(N(Cc2ccc(-c3ccc(N(C)C)cc3)cc2)C(=O)C2CCCCC2)c1]

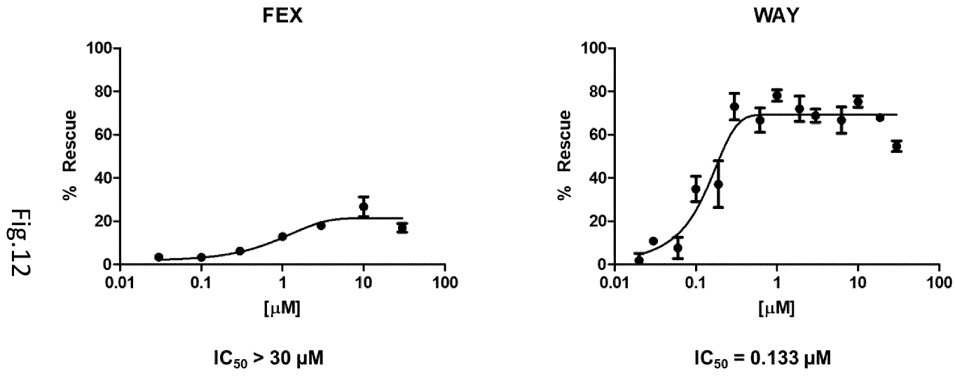

HEK293 cells expressing ΔCR PrP were cultured at ˜60% confluence in 24-well plates on day 1. On day 2, cells were treated with 500 μg/mL of Zeocin and/or individual FXR agonists at different concentrations (0.03-30 μM) for 72 hr. Medium (containing fresh Zeocin and/or FXR agonists) was replaced every 24 hr. On day 5, cell medium was removed and cells were incubated with 1 mg/mL of 3-(4,5-dimethylthiazol-2-yl)-2,5-diphenyltetrazolium bromide (MTT) in PBS for 30 min at 37° C. to evaluate cell viability. Interestingly, the FXR agonist WAY-362450 rescued ΔCR PrP-dependent citotoxicity in a dose-dependent fashion, with an inhibitory concentration at 50% (IC₅₀) value in the sub-micromolar range (FIG. 12). Importantly, the FXR agonist Fexaramine showed a much lower effect, possibly reflecting a different activity of the two agonists against specific FXR isoforms and/or recruiting also different coactivators. Collectively, these results establish a direct pharmacological connection between mutant PrP toxicity and the activity of the FXR receptor, and suggest that this receptor could be the target of SM compounds.

SM231 mediates FXR gene transcriptional activity in murine hepatocytes. Mouse primary hepatocytes were isolated from 6-8-week-old C57Bl6/J wild-type male mice (from Charles River). 3×10⁶ prymary hepatocytes were stimulated with increasing concentrations of SM231 or WAY-362450, a potent and selective Farnesoid X receptor (FXR) agonist for 4 or 12 hours. The expression of FXR (nr1 h4) and the FXR target gene Nr0b2, was evaluated by RT-qPCR using specific primers.

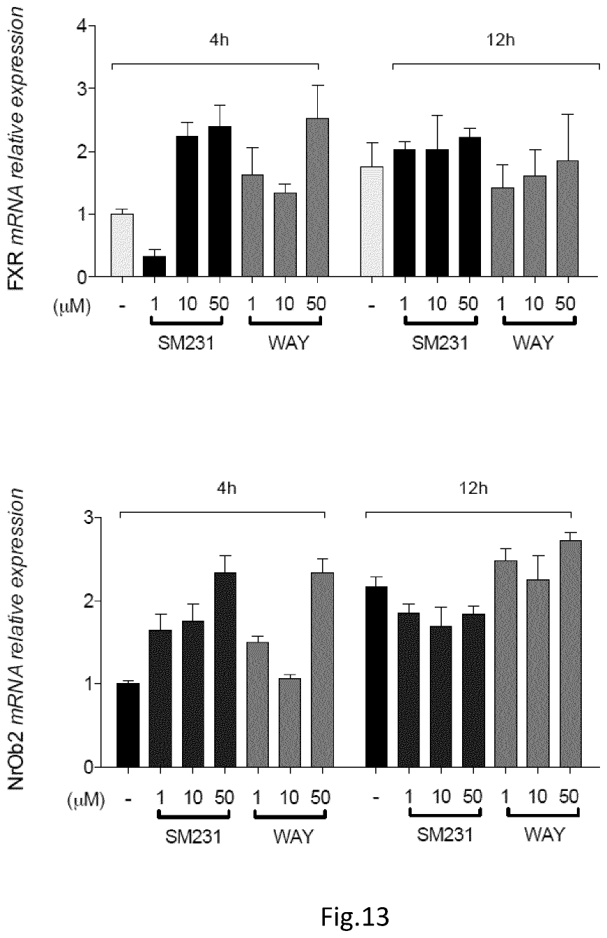

In this experiment, similarly to the reference agonist WAY-362450, SM231 promoted significant FXR transcriptional activity in these cells, specifically three hours after treatment (FIG. 13). The effects were lost after 6 hours of activation for both molecules. These data suggest that SM231 may act in cells as an FXR receptor agonist.

### REFERENCES

1 Chiti, F. & Dobson, C. M. Protein Misfolding, Amyloid Formation, and Human Disease: A Summary of Progress Over the Last Decade. Annu Rev Biochem 86, 27-68, doi:10.1146/annurev-biochem-061516-045115 (2017).
2 Selkoe, D. J. & Hardy, J. The amyloid hypothesis of Alzheimer's disease at 25 years. EMBO Mol Med 8, 595-608, doi:10.15252/emmm.201606210 (2016).
3 Walsh, D. M. et al. Naturally secreted oligomers of amyloid beta protein potently inhibit hippocampal long-term potentiation in vivo. Nature 416, 535-539, doi:10.1038/416535a (2002).
4 Lauren, J., Gimbel, D. A., Nygaard, H. B., Gilbert, J. W. & Strittmatter, S. M. Cellular prion protein mediates impairment of synaptic plasticity by amyloid-beta oligomers. Nature 457, 1128-1132, doi:10.1038/nature07761 (2009).
5 Prusiner, S. B. Prions. Proc Natl Acad Sci USA 95, 13363-13383, doi:10.1073/pnas.95.23.13363 (1998).
6 Mallucci, G. et al. Depleting neuronal PrP in prion infection prevents disease and reverses spongiosis. Science 302, 871-874, doi:10.1126/science.1090187 (2003).
7 Brandner, S. et al. Normal host prion protein necessary for scrapie-induced neurotoxicity. Nature 379, 339-343, doi:10.1038/379339a0 (1996).
8 Biasini, E., Turnbaugh, J. A., Unterberger, U. & Harris, D. A. Prion protein at the crossroads of physiology and disease. Trends Neurosci 35, 92-103, doi:10.1016/j.tins.2011.10.002 (2012).
9 Chung, E. et al. Anti-PrPC monoclonal antibody infusion as a novel treatment for cognitive deficits in an Alzheimer's disease model mouse. BMC Neurosci 11, 130, doi:10.1186/1471-2202-11-130 (2010).
10 Gimbel, D. A. et al. Memory impairment in transgenic Alzheimer mice requires cellular prion protein. J Neurosci 30, 6367-6374, doi:10.1523/JNEUROSCI.0395-10.2010 (2010).
11 Fluharty, B. R. et al. An N-terminal fragment of the prion protein binds to amyloid-beta oligomers and inhibits their neurotoxicity in vivo. J Biol Chem 288, 7857-7866, doi:10.1074/jbc.M112.423954 (2013).
12 Um, J. W. et al. Alzheimer amyloid-beta oligomer bound to postsynaptic prion protein activates Fyn to impair neurons. Nat Neurosci 15, 1227-1235, doi:10.1038/nn.3178 (2012).
13 Ferreira, D. G. et al. alpha-synuclein interacts with PrP(C) to induce cognitive impairment through mGluR5 and NMDAR2B. Nat Neurosci 20, 1569-1579, doi:10.1038/nn.4648 (2017).
14 Aulic, S. et al. alpha-Synuclein Amyloids Hijack Prion Protein to Gain Cell Entry, Facilitate Cell-to-Cell Spreading and Block Prion Replication. Sci Rep 7, 10050, doi:10.1038/s41598-017-10236-x (2017).
15 Resenberger, U. K., Winklhofer, K. F. & Tatzelt, J. Cellular prion protein mediates toxic signaling of amyloid beta. Neurodegener Dis 10, 298-300, doi:10.1159/000332596 (2012).
16 Linden, R. The Biological Function of the Prion Protein: A Cell Surface Scaffold of Signaling Modules. Front Mol Neurosci 10, 77, doi:10.3389/fnmol.2017.00077 (2017).
17 Manni, G. et al. The cellular prion protein beyond prion diseases. Swiss Med Wkly 150, w20222, doi:10.4414/smw.2020.20222 (2020).
18 Ballerini, C. et al. Functional implication of cellular prion protein in antigen-driven interactions between T cells and dendritic cells. J Immunol 176, 7254-7262, doi:10.4049/jimmunol.176.12.7254 (2006).
19 Esterhazy, D. et al. Classical dendritic cells are required for dietary antigen-mediated induction of peripheral T(reg) cells and tolerance. Nat Immunol 17, 545-555, doi:10.1038/ni.3408 (2016).
20 Massignan, T. et al. A novel, drug-based, cellular assay for the activity of neurotoxic mutants of the prion protein. J Biol Chem 285, 7752-7765, doi:10.1074/jbc.M109.064949 (2010).
21 Imberdis, T. et al. Identification of Anti-prion Compounds using a Novel Cellular Assay. J Biol Chem 291, 26164-26176, doi:10.1074/jbc.M116.745612 (2016).
22 Massignan, T. et al. A Small-Molecule Inhibitor of Prion Replication and Mutant Prion Protein Toxicity. ChemMedChem 12, 1286-1292, doi:10.1002/cmdc.201700302 (2017).
23 Solomon, I. H., Biasini, E. & Harris, D. A. Ion channels induced by the prion protein: mediators of neurotoxicity. Prion 6, 40-45, doi:10.4161/pri.6.1.18627 (2012).
24 Solomon, I. H. et al. An N-terminal polybasic domain and cell surface localization are required for mutant prion protein toxicity. J Biol Chem 286, 14724-14736, doi:10.1074/jbc.M110.214973 (2011).
25 Massignan, T., Biasini, E. & Harris, D. A. A Drug-Based Cellular Assay (DBCA) for studying cytotoxic and cytoprotective activities of the prion protein: A practical guide. Methods 53, 214-219, doi:10.1016/j.ymeth.2010.11.005 (2011).
26 Massignan, T. et al. A cationic tetrapyrrole inhibits toxic activities of the cellular prion protein. Sci Rep 6, 23180, doi:10.1038/srep23180 (2016).
27 Foliaki, S. T. et al. Prion acute synaptotoxicity is largely driven by protease-resistant PrPSc species. PLoS Pathog 14, e1007214, doi:10.1371/journal.ppat.1007214 (2018).
28 Foliaki, S. T. et al. Early existence and biochemical evolution characterise acutely synaptotoxic PrPSc. PLoS Pathog 15, e1007712, doi:10.1371/journal.ppat.1007712 (2019).
29 Biggi, S. et al. Identification of compounds inhibiting prion replication and toxicity by removing PrP(C) from the cell surface. J Neurochem 152, 136-150, doi:10.1111/jnc.14805 (2020).
30 Stincardini, C. et al. An antipsychotic drug exerts anti-prion effects by altering the localization of the cellular prion protein. PLoS One 12, e0182589, doi:10.1371/journal.pone.0182589 (2017).
31 Barmada, S., Piccardo, P., Yamaguchi, K., Ghetti, B. & Harris, D. A. GFP-tagged prion protein is correctly localized and functionally active in the brains of transgenic mice. Neurobiol Dis 16, 527-537, doi:10.1016/j.nbd.2004.05.005 (2004).
32 Ivanova, L., Barmada, S., Kummer, T. & Harris, D. A. Mutant prion proteins are partially retained in the endoplasmic reticulum. J Biol Chem 276, 42409-42421, doi:10.1074/jbc.M106928200 (2001).
33 Berman, H. M., Battistuz, T., Bhat, T. N., Bluhm, W. F., Bourne, P. E., Burkhardt, K., Feng, Z., Gilliland, G. L., Iype, L., Jain, S., Fagan, P., Marvin, J., Padilla, D., Ravichandran, V, Schneider, B., Thanki, N., Weissig, H., Westbrook, J. D., Zardecki, C. The protein data bank, Acta Crystallogr. Sect. D Biol. Crystallogr. 58 (2002) 899e907, https://doi.org/10.1107/S0907444902003451
34 Friesner, R. A., Banks, J. L., Murphy, R. B., Halgren, T. A., Klicic, J. J., Mainz, D. T., Repasky, M. P., Knoll, E. H., Shelley, M., Perry, J. K., Shaw, D. E., Francis, P., Shenkin P. S. Glide: a new approach for rapid, accurate docking and scoring. 1.
35 Method and assessment of docking accuracy, J. Med. Chem. 47 (2004) 1739e1749, https://doi.org/10.1021/jm0306430
35 Nicoll, A. J., Clare R. Trevitt, M., Tattum, H., Risse, E., Quarterman, E., Avila Ibarra, A., Wright, C., Graham, S. J., Sessions, R. B., Farrow, M., Waltho, J. P., Clarke, A. R. Collinge, J. Pharmacological chaperone for the structured domain of human prion protein—PNAS, Oct. 12, 2010, vol. 107, no. 41; https://doi.org/10.1073/pnas.1009062107.
36 Lu, T. T., Makishima, M., Repa, J. J., Schoonjans, K., Kerr, T. A., Auwerx, J., Mangelsdorf, D. J. Molecular basis for feedback regulation of bile acid synthesis by nuclear receptors. Mol Cell. 2000 September; 6(3):507-15.
37 Das, B., Venkateswarlu, K., Mahender, G., Mahender, I. A simple and efficient method for α-bromination of carbonyl compounds using N-bromosuccinimide in the presence of silica-supported sodium hydrogen sulfate as a heterogeneous catalyst. Tetrahedron Letters (2005), 46(17), 3041-3044

1. Method of treating of a neurodegeneratie disease or an immune disease in a patient in need thereof with a compound of general Formula (I): 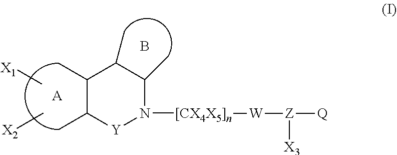
wherein
A is a benzene ring or a five- or six heteroaromatic ring;
B is a benzene ring of general structure: 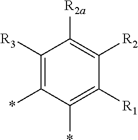
or B is a five- or six membered heteroaromatic ring optionally substituted by one or more substituents each independently selected from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino;
W is C(═O), C(═S), CH₂ or is absent;
Y is selected from CH₂, SO₂, SO, S, C(═O), PO₂ and NR₄;
Z is N or CH;
X₁ and X₂ are each independently selected in each instance from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁-4alkylamino, OH, pyrrolidine, piperidine, morpholine, piperazine, N-methylpiperazine;
X₃ is hydrogen, methyl, ethyl, isopropyl or benzyl;
X₄ and X₅ are independently selected in each instance from hydrogen, C₁₋₃alkyl, haloalkyl, halogen, cycloalkyl, amino, hydroxy, cyano, nitro; n is 0, 1, 2, 3, 4;
or residues X₃ and X₄ taken together represent a single bond or a C₁₋₄alkanediyl, said single bond or said C₁₋₄alkanediyl forming together with the bridging atoms to which they are respectively linked a 5 or 6 membered heterocyclic ring;
R₁, R₂, R₂ₐ and R₃ are each independently selected from hydrogen, halogen, nitro, cyano, hydroxy, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, OC₁₋₄alkylamino, SC₁₋₄alkyl;
R₄ is selected from hydrogen, C₁₋₄alkyl, C₁₋₄aminoalkyl, C₁₋₄hydroxyalkyl, C₁₋₄nitroalkyl, C₁₋₄thioalkyl, C₁₋₆haloalkyl;
Q is selected from C₁₋₈ alkyl, C₁₋₈ alkenyl, cycloalkyl, heterocycloalkyl, aryl ring, heteroaromatic ring, wherein:
the C₁₋₈ alkyl is optionally substituted with hydroxy, OC₁₋₄alkyl, NHC₁₋₄alkyl, N(C₁₋₄alkyl)₂, NH(C═O)C₁₋₄alkyl, aryl, heteroaryl, heterocycloalkyl, cycloalkyl, cycloalkenyl, each of said aryl, heteroaryl, heterocycloalkyl, cycloalkyl, cycloalkenyl being optionally substituted with methyl, halogen, hydroxy;
the cycloalkyl and the heterocycloalkyl are each optionally substituted with OH, OSO₂R₅, C₁₋₃alkyl, NR₆R₇, wherein:
R₅ is selected from hydrogen, phenyl, heteroaryl, aminophenyl and nitrophenyl; and wherein
R₆ and R₇ are each independently selected from H, methyl, C(═O)CH₃, SO₂CH₃;
the aryl ring or the heteroaromatic ring are each optionally substituted with one or more substituents selected from halogen, nitro, cyano, thiol, C₁₋₄alkyl, haloalkyl, O-haloalkyl, OC₁₋₄alkyl, NH₂, NHSO₂C₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino;
and any stereoisomer, pharmaceutically acceptable salt, hydrate, solvate thereof,
wherein said method comprises
administering to said patient a pharmaceutical effective amount of said compound.

2. (canceled)

3. The method according to claim 1 wherein:
A is benzene; and/or
Y is SO₂; and/or
W is C(═O) or CH₂; and/or
Z is N; and/or
X₄ and X₅ are H.

4. The method according to claim 1, wherein the compound has general formula (II):

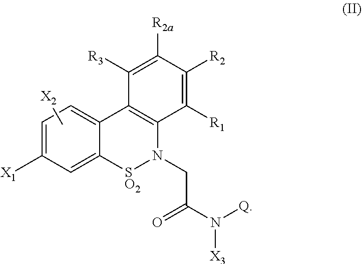

5. The method according to claim 1, the compound being:

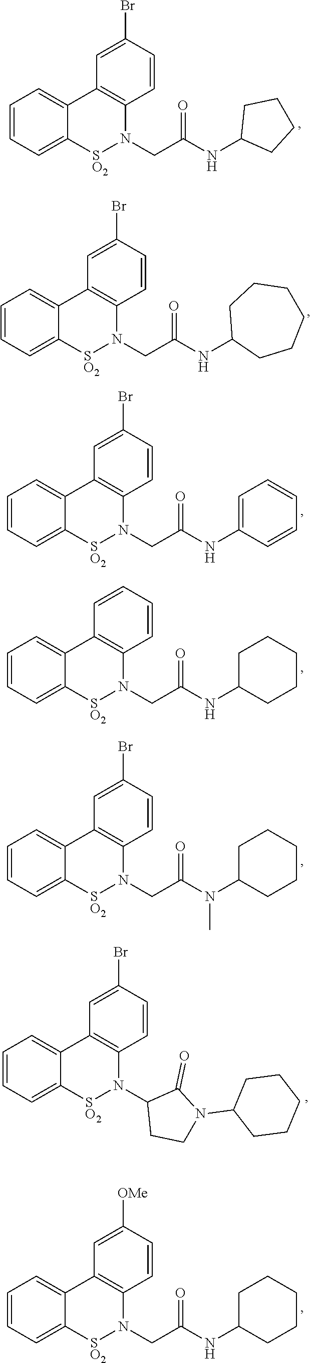

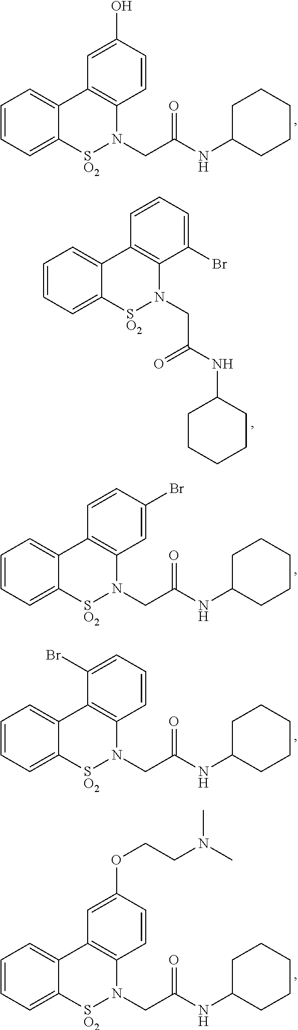

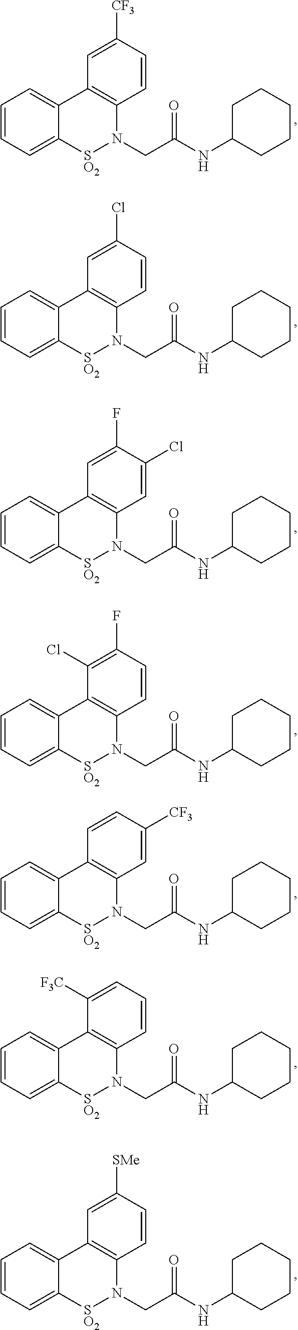

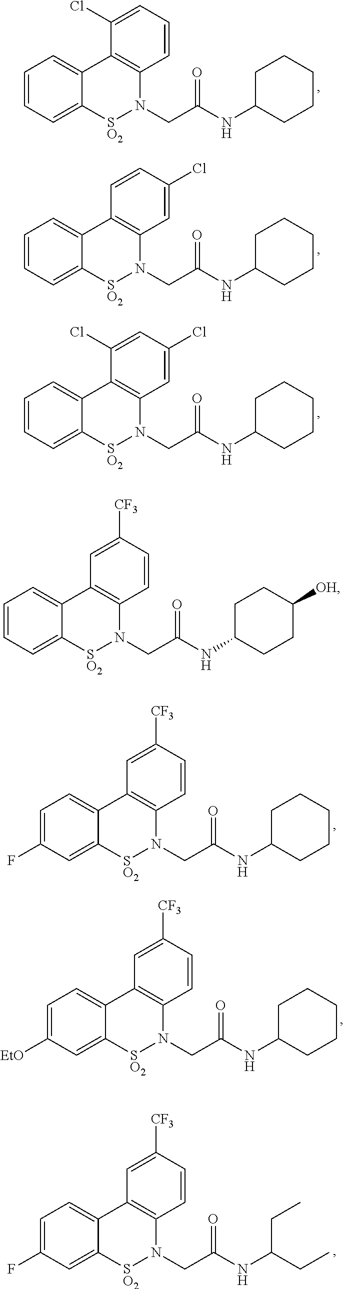

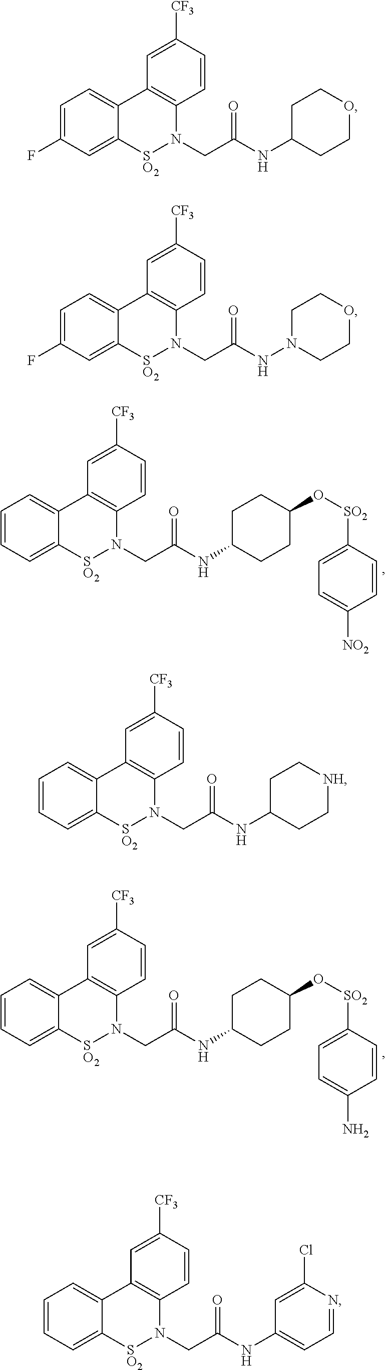 [SMILES: O=C(CN1c2ccc(C(F)(F)F)cc2-c2ccc(F)cc2S1(=O)=O)NC1CCOCC1 ; O=C(CN1c2ccc(C(F)(F)F)cc2-c2ccc(F)cc2S1(=O)=O)NN1CCOCC1 ; O=C(CN1c2ccc(C(F)(F)F)cc2-c2ccccc2S1(=O)=O)N[C@H]1CC[C@H](OS(=O)(=O)c2ccc([N+](=O)[O-])cc2)CC1 ; O=C(CN1c2ccc(C(F)(F)F)cc2-c2ccccc2S1(=O)=O)NC1CCNCC1 ; Nc1ccc(S(=O)(=O)O[C@H]2CC[C@H](NC(=O)CN3c4ccc(C(F)(F)F)cc4-c4ccccc4S3(=O)=O)CC2)cc1 ; O=C(CN1c2ccc(C(F)(F)F)cc2-c2ccccc2S1(=O)=O)Nc1ccnc(Cl)c1]

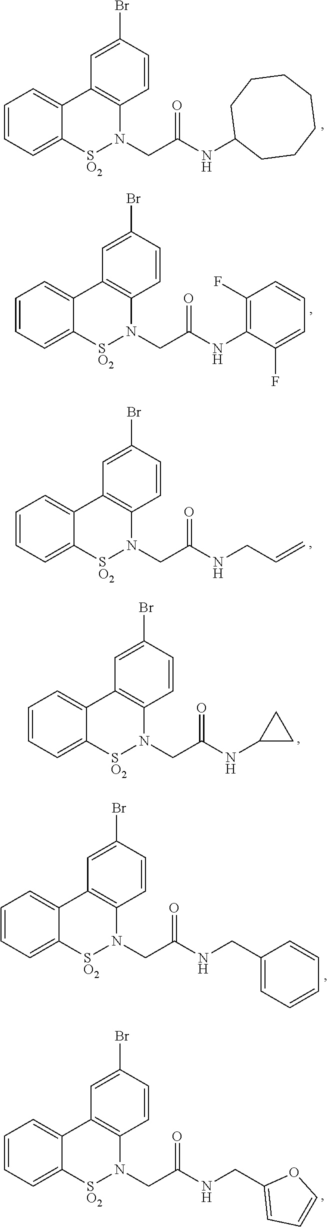

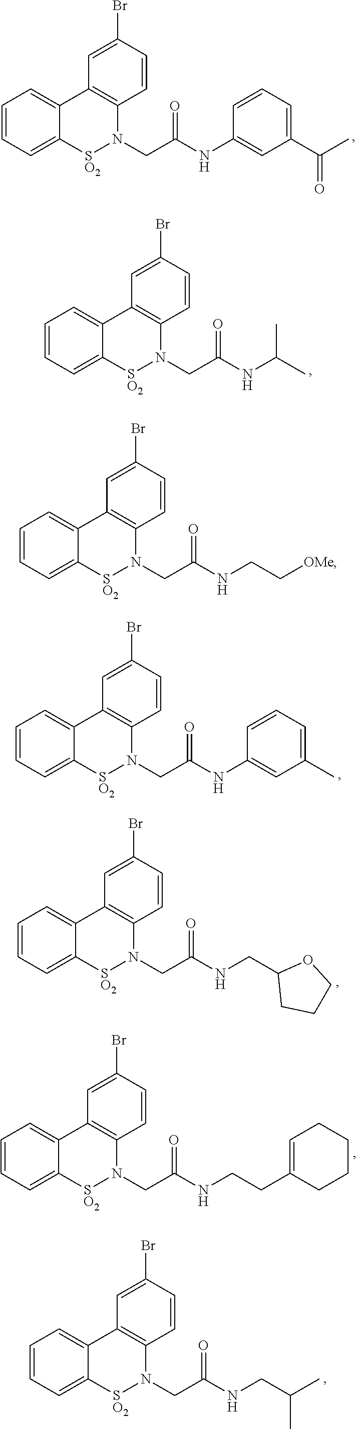

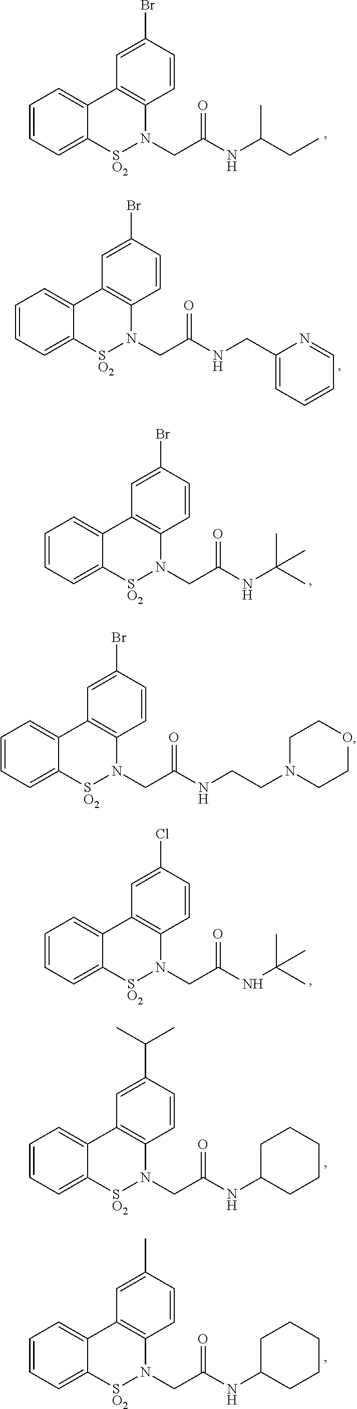

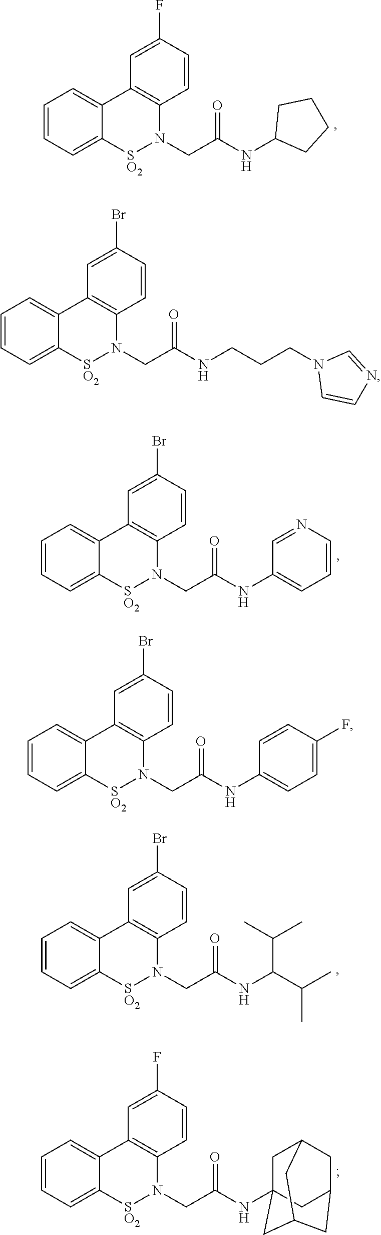 [SMILES: O=C(CN1c2ccc(F)cc2-c2ccccc2S1(=O)=O)NC1CCCC1 ; O=C(CN1c2ccc(Br)cc2-c2ccccc2S1(=O)=O)NCCCn1ccnc1 ; O=C(CN1c2ccc(Br)cc2-c2ccccc2S1(=O)=O)Nc1cccnc1 ; O=C(CN1c2ccc(Br)cc2-c2ccccc2S1(=O)=O)Nc1ccc(F)cc1 ; CC(C)C(NC(=O)CN1c2ccc(Br)cc2-c2ccccc2S1(=O)=O)C(C)C ; O=C(CN1c2ccc(F)cc2-c2ccccc2S1(=O)=O)NC12CC3CC(CC(C3)C1)C2]

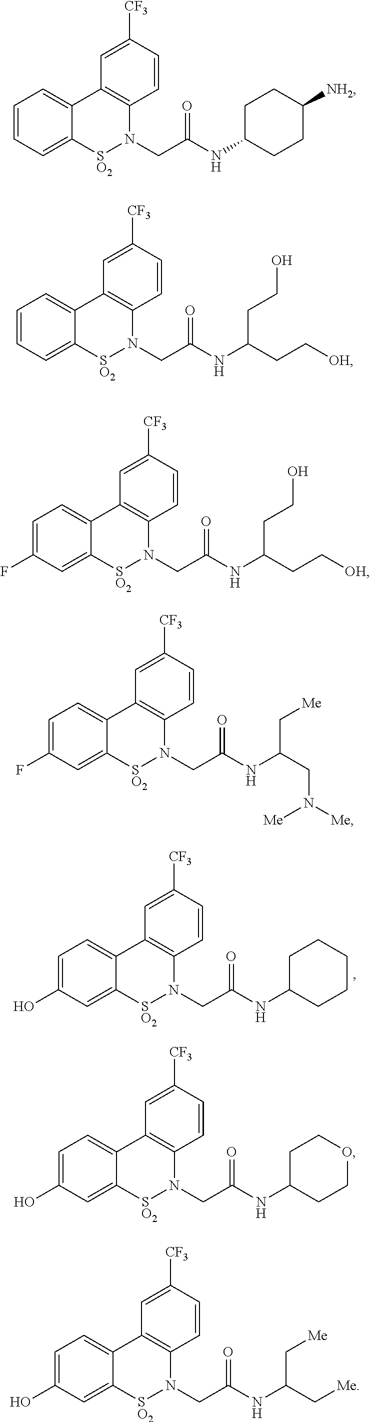

6. The method according to claim 1, the compound being:

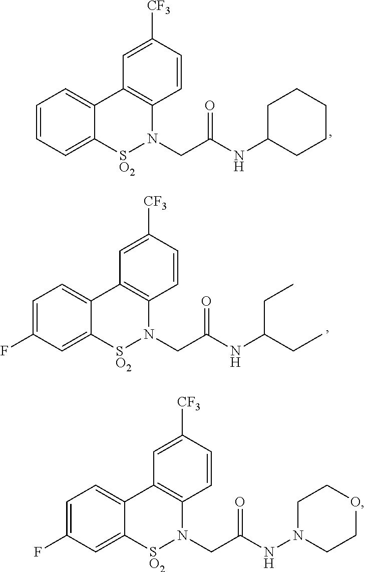 [SMILES: O=C(CN1c2ccc(C(F)(F)F)cc2-c2ccccc2S1(=O)=O)NC1CCCCC1 ; CCC(CC)NC(=O)CN1c2ccc(C(F)(F)F)cc2-c2ccc(F)cc2S1(=O)=O ; O=C(CN1c2ccc(C(F)(F)F)cc2-c2ccc(F)cc2S1(=O)=O)NN1CCOCC1]

7. (canceled)

8. A compound of general Formula (III): 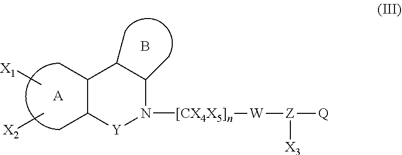
wherein
A is a benzene ring or a five- or six heteroaromatic ring;
B is a benzene ring of general structure: 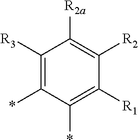
wherein:
R₁, R₂ and R₃ are each independently selected from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₆alkyl, OC₁₋₄alkylamino, hydroxy, SC₁₋₄alkyl;
R₂ₐ is hydrogen, CF₃, F, OH, OC₁₋₄alkyl, SC₁₋₄alkyl, OC₁₋₄alkylamino
with the proviso that:
if R₂ₐ is hydrogen or F, then R₂ and R₃ are each independently selected from F, Cl, Br, CF₃, OMe, OH; or
if R₁ is halogen, then R₂ₐ is hydrogen and R₂ and R₃ are each independently selected from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₆alkyl, OC₁₋₄alkylamino, hydroxy, SC₁₋₄alkyl;
or B is a five- or six membered heteroaromatic ring optionally substituted by one or more substituents each independently selected from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₆alkyl, hydroxy, SC₁₋₄alkyl, OC₁₋₄alkylamino;
W is C(═O);
Y is selected from CH₂, SO₂, SO, S, C(═O), PO₂, and NR₄;
Z is N or CH;
X₁ and X₂ are each independently selected in each instance from hydrogen, halogen, nitro, cyano, thiol, C₁₋₄alkyl, haloalkyl, O-haloalkyl, OC₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino, OH, pyrrolidine, piperidine, morpholine, piperazine, N-methylpiperazine;
X₃ is hydrogen, methyl, ethyl, isopropyl or benzyl;
X₄ and X₅ are independently selected in each instance from hydrogen, C₁₋₃alkyl, haloalkyl, halogen, cycloalkyl, amino, hydroxy, cyano, nitro; n is 0, 1, 2, 3, 4;
or residues X₃ and X₄ taken together represent a single bond or a C₁₋₄alkanediyl, said single bond or said C₁₋₄alkanediyl forming together with the bridging atoms to which they are respectively linked a 5 or 6 membered heterocyclic ring;
R₄ is selected from hydrogen, C₁₋₄alkyl, C₁₋₄aminoalkyl, C₁₋₄hydroxyalkyl, C₁₋₄nitroalkyl, C₁₋₄thioalkyl, C₁₋₆haloalkyl;
Q is selected from C₁₋₈ alkyl, C₁₋₈ alkenyl, cycloalkyl, heterocycloalkyl, aryl ring, heteroaromatic ring, wherein:
the C₁₋₈ alkyls is optionally substituted with hydroxy, OC₁₋₄alkyl, NHC₁₋₄alkyl, N(C₁₋₄alkyl)₂, NH(C═O)C₁₋₄alkyl, aryl, heteroaryl, heterocycloalkyl, cycloalkyl, cycloalkenyl, each of said aryl, heteroaryl, heterocycloalkyl, cycloalkyl, cycloalkenyl being optionally substituted with methyl, halogen, hydroxy;
the cycloalkyl and the heterocycloalkyl are each optionally substituted with OH, OSO₂R₅, C₁₋₃alkyl, NR₆R₇, wherein:
R₅ is selected from hydrogen, phenyl, heteroaryl, aminophenyl and nitrophenyl; and wherein
R₆ and R₇ are each independently selected from H, methyl, C(═O)CH₃, SO₂CH₃;
the aryl ring or the heteroaromatic ring are each optionally substituted with one or more substituents selected from halogen, nitro, cyano, thiol, C₁₋₄alkyl, haloalkyl, O-haloalkyl, OC₁₋₄alkyl, NH₂, NHSO₂C₁₋₄alkyl, NHC₁₋₄alkyl, C(═O)C₁₋₆alkyl, C(═O)OC₁₋₆alkyl, C(═O)NHC₁₋₄alkyl, hydroxy, SC₁₋₄alkyl, OC₁₋₄alkylamino;
and any stereoisomer, pharmaceutically acceptable salt, hydrate, solvate thereof.

9. The compound of formula (III) according to claim 8 wherein:
A is benzene; and/or
Y is SO₂; and/or
Z is N; and/or
X₄ and X₅ are H.

10. The compound of formula (III) according to claim 8 being:

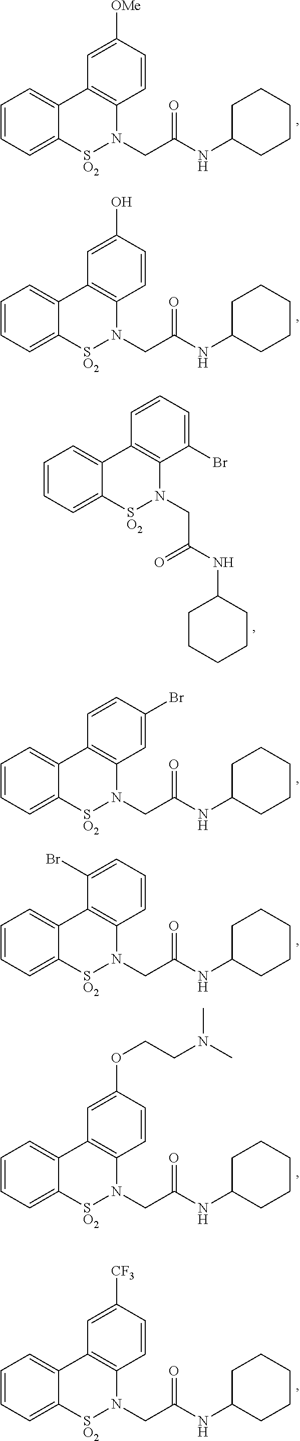

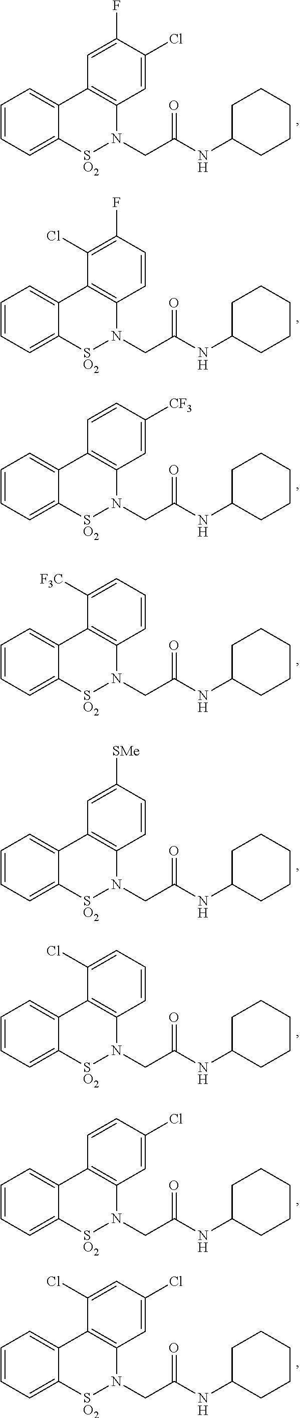

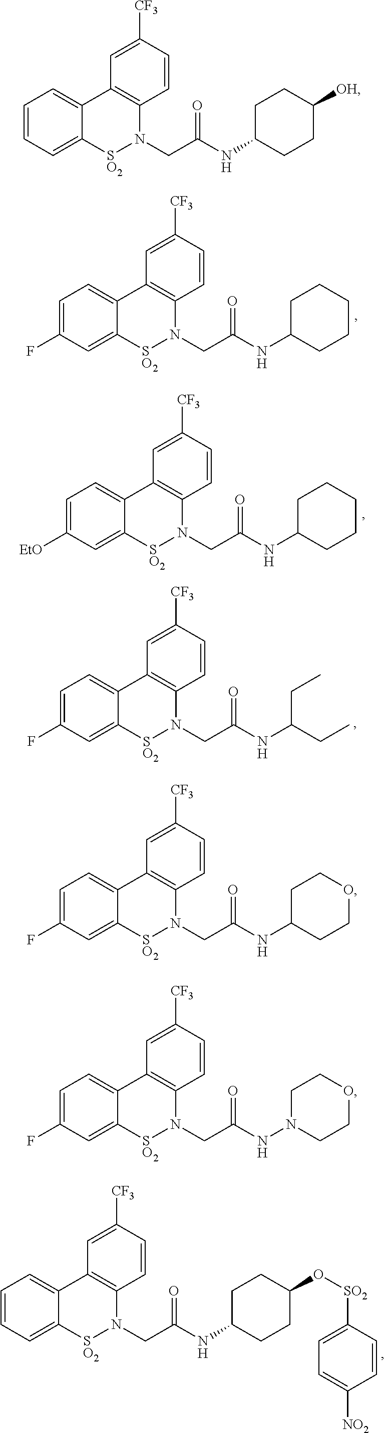

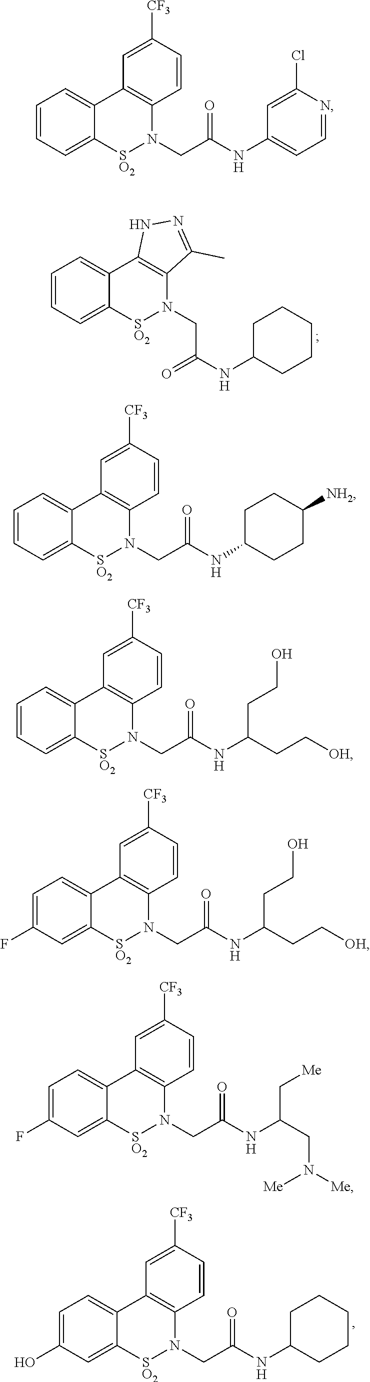

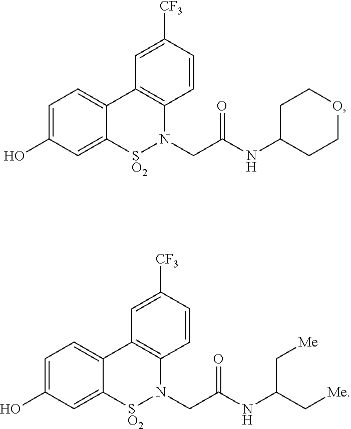 [SMILES: O=C(CN1c2ccc(C(F)(F)F)cc2-c2ccc(O)cc2S1(=O)=O)NC1CCOCC1 ; CCC(CC)NC(=O)CN1c2ccc(C(F)(F)F)cc2-c2ccc(O)cc2S1(=O)=O]

11.-12. (canceled)

13. The method according to claim 18, wherein said compound modulates activity of cellular prion protein (PrPC).

14. A pharmaceutical composition comprising at least one compound as defined in claim 8, alone or in combination with at least one further active compound, and at least one pharmaceutically acceptable excipient.

15. (canceled)

16. A process for the synthesis of a compound of general formula (III) according to claim 6, wherein A is benzene and Y is SO2, comprising the following steps:
a) reacting a compound of formula 1a with an aromatic or heteroaromatic amine of formula 1b, in the presence of a solvent like dichlorometane and an amine like pyridine, trimethylamine, diethylisopropylamine, to give a compound of formula 2a 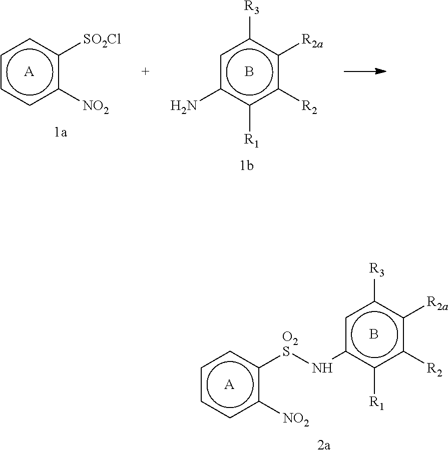
b) reducing the nitro group of compound of formula 2a to an amino group by hydrogenation in the presence of Raney-Nichel catalyst or with SnCl2·2H2O under appropriate conditions, to obtain a compound of formula 3a: 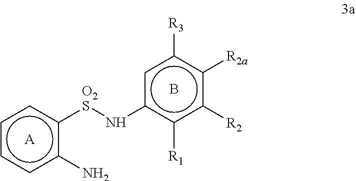
c) converting compound 3a into a compound of formula 5a: 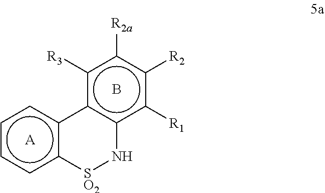
by a first step comprising reaction with NaNO₂, NaOH and HCl under appropriate conditions, and a second step employing Cu powder and DMSO as solvent at room temperature;
d) converting compound of formula 5a into a compound of formula (I) or of formula (II), wherein the reaction comprises at least one of the following step:
reacting 5a with an alkylating agent of formula hal-(CH₂)ₙ—C(═O)OEt or with an alkylating agent of formula hal-(CH₂)ₙ-Q wherein hal is bromine or chlorine;
treating with an amine of formula Q-NHX₃ under microwawe irradiation and neat conditions;
coupling with an amine of formula Q-NHX₃ in the presence of a condensing agents such as TBTU in CH₂Cl₂ and DIPEA or using SOCl₂ as chlorinating agent.

17. The method according to claim 1, wherein said neurodegenerative disease or said immune disease is selected from the group consisting of Alzheimer's Disease, Prion Disease, Multiple Sclerosis, Autoimmune Encephalitis, Parkinsons' Disease, Inflammatory Bowel Disease and Crohn's disease.

18. The method of claim 17, wherein said neurodegenerative disease is Prion Disease,
provided that compound 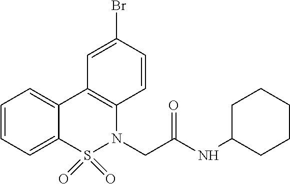 [SMILES: O=C(CN1c2ccc(Br)cc2-c2ccccc2S1(=O)=O)NC1CCCCC1]
is not included.

19. The compound of formula III according to claim 8 being: 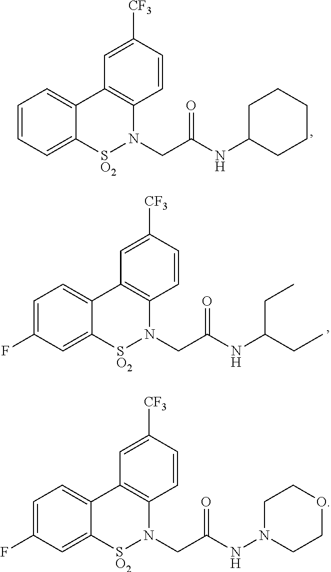 [SMILES: O=C(CN1c2ccc(C(F)(F)F)cc2-c2ccccc2S1(=O)=O)NC1CCCCC1 ; CCC(CC)NC(=O)CN1c2ccc(C(F)(F)F)cc2-c2ccc(F)cc2S1(=O)=O ; O=C(CN1c2ccc(C(F)(F)F)cc2-c2ccc(F)cc2S1(=O)=O)NN1CCOCC1]


SEQUENCE LISTING (2 sequences)

SEQ ID NO: 1 - protein, 209 aa, Homo sapiens
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSS

SEQ ID NO: 2 - protein, 21 aa, Homo Sapiens
MEVGWYRSPFSRVVHLYRNGK